(19) United States
(12) Patent Application Publication
(10) Pub. No.: US 2022/0101951 A1
(43) Pub. Date: Mar. 31, 2022

(54) Systems and Methods for Homogenization of Disparate Datasets

(71) Applicant: Tempus Labs, Inc., Chicago, IL (US)

(72) Inventors: Talal Ahmed, Edison, NJ (US); Raphael Pelossof, New York, NY (US); Stephane Wenric, New York, NY (US); Mark Carty, Brooklyn, NY (US)

(21) Appl. No.: 17/548,084

(22) Filed: Dec. 10, 2021

Related U.S. Application Data
(63) Continuation of application No. 17/405,025, filed on Aug. 18, 2021.
(60) Provisional application No. 63/067,748, filed on Aug. 19, 2020.
(60) Provisional application No. 63/203,804, filed on Jul. 30, 2021.

(51) Int. Cl. G16B 50/20; G06N 3/12; G16B 40/00; G06K 9/62

(52) U.S. Cl. CPC G16B 50/20; G06N 3/12; G06K 9/6262; G06K 9/6256; G16B 40/00

(57) ABSTRACT

A method for transferring a dataset-specific nature of a first dataset with sequencing results for a first plurality of specimen to a second dataset with sequencing results for a second plurality of specimen includes receiving a first set of adaptation factors of the first dataset that include two or more eigenvectors, where the sequencing cannot be reconstructed from the first set of adaptation factors without access to the first dataset. The method also includes generating a second set of adaptation factors of the second dataset that include two or more eigenvectors of the second dataset. The method also includes generating an adapted second dataset by adapting the dataset-specific nature of the second dataset to the dataset-specific nature of the second dataset based at least in part on the first and second sets of adaptation factors, and providing the adapted second dataset to the first entity.

### CROSS-REFERENCE TO RELATED APPLICATIONS

The present application is a continuation of U.S. patent application Ser. No. 17/405,025, filed Aug. 18, 2021, which claims the benefit of priority under 35 U.S.C. § 119(e) to U.S. Provisional Application No. 63/067,748, filed Aug. 19, 2020, and U.S. Provisional Application No. 63/203,804, filed Jul. 30, 2021, the content of which are incorporated herein by reference in their entireties.

### FIELD OF THE INVENTION

The present disclosure relates to computer-implemented methods and systems for expressing datasets representing data bias due to differing populations, capturing methodologies, or phenomenon in a uniform format, and more specifically to optimizing differing high-dimensional datasets for an artificial intelligence engine.

### BACKGROUND

The advent of high-throughput gene expression profiling has powered the development of sophisticated molecular models to capture complex biological patterns. To ensure the generalization of molecular patterns across independent studies, molecular predictors require validation across platforms and laboratories. However, the transfer of predictors across laboratories still remains a technical obstacle. Batch-specific effects that dominate the biological signal exist between different technologies, laboratories and even library preparation protocols within the same laboratory. Furthermore, often these inter-institutional datasets are siloed due to human subject privacy concerns. There is an unmet need for a technology that enables the transfer of molecular predictors across labs in a privacy-preserving manner such that sample-level patient data is not transferred.

Correction of batch-specific biases in RNA-Seq datasets has been an active field of research in the past two decades. Numerous methods are proposed to correct batch effects, and these mostly fall into two categories: batch integration and batch correction. Batch integration entails joint embedding of batch-biased expression data in a shared embedding space where batch variations are minimized. Batch correction removes batch biases in the gene expression space, harmonizing batch-biased dataset(s) to a reference dataset. For batch correction, we refer to the reference dataset as the target and the batch-biased dataset as the source. Since the reference remains unchanged in batch correction, the asymmetry enables the transfer (application) of models trained on reference dataset to batch-corrected dataset(s).

Machine learning models such as disease or subtype classifiers are often developed using assay data such as RNA expression data. Batch integration methods may not be suitable for transferring classifiers trained on gene expression datasets, since integration methods do not necessarily output expression profiles. These include methods based on gene-wise linear models like Limma, mutually nearest neighbors (MNNs) like MNN Correct and Scanorama, mutually nearest clusters (MNCs) like ScGen, pseudoreplicates like ScMerge, and multi-batch clusters like Harmony. In contrast, batch correction methods can correct a source library to a target reference library, like Combat and Seurat3, and thus can be used for transferring classifiers across expression datasets.

Prior integration and correction methods require full sample-level access for integration or correction of datasets. Therefore, the transfer of molecular predictors between laboratories necessitates the transfer of patient-level training data for the molecular predictor. This data access requirement can inhibit the transfer of models between laboratories, given transfer of data may not be possible due to data ownership, GDPR, or similar regulations.

Artificial intelligence engines implementing machine learning algorithms are critical to advancing computer science across fields including applications of medicine and engineering. One caveat often faced by computer scientists and engineers developing artificial intelligence engines is that the datasets they are training these engines with may inherently express bias to one characteristic of the data or another. When the model is applied to new sources of data that do not express bias to that characteristic, or express bias in a different way, the engine's performance may decline or fail altogether.

Biases can be difficult to identify and eliminate. In one system, biases may exist because the sampling of training data are insufficiently balanced across representative classes. In another system, biases may exist due to data that is or is not present. Instances of unexpected bias are disclosed in “Dissecting racial bias in an algorithm used to manage the health of populations” by Ziad Obermeyer, Brian Powers, Christine Vogeli, Sendhil Mullainathan and “How Algorithms Discriminate Based on Data They Lack: Challenges, Solutions, and Policy Implications” by Betsy Anne Williams, Catherine F. Brooks and Yotam Shmargad.

Genomic sequencing results may be biased depending on the equipment, methods, and other characteristics of the laboratory conducting the sequencing, or the types of disease states sequencing targets. When results of one laboratory are combined with results of other laboratories, the bias from each laboratory may impact the manner in which the data is synthesized overall, such as when the data is used to develop and/or validate one or more artificial intelligence engines.

Recent advances in transcriptomic technology have resulted in the emergence of several publicly available databases of molecular data. Although these datasets provide excellent opportunities for researchers to increase their cohort sizes for efficient statistical learning, several challenges remain to integrate patients' molecular data. As different datasets tend to exhibit domain shifts and batch biases across various sequencing platforms and library preparation methods, correcting for these biases while preserving molecular structures and critical clinical associations, such as cancer subtypes or survival risk, constitutes an important issue to be addressed as part of integrating large-scale sequencing datasets, such as cancer sequencing datasets.

Researchers curating engines trained from one dataset may find that the resulting models are not transferable to other datasets, such as other datasets which were not employed for model training, due to the model fitting to those different domain shifts, target shifts, or biases. What is needed is a system and method for learning the differences between two datasets and effecting a transformation for aligning the datasets to enable a model trained on one dataset to be applied to a second dataset.

Different entities, such as laboratories, research institutions, health institutions, or pharmaceutical companies may desire to collaborate together while preserving the privacy of their internally curated data. Unable to accomplish their goals without directly sharing data, these entities need an effective way to work together while maintaining data privacy.

The systems and methods described herein cure the aforementioned defects and allow artificial intelligence engines trained on one dataset to be applied to any other dataset following an adaptation process which identifies the nature or biases, domain shifts, covariate shifts, or other dataset specific phenomenon, where the distribution of samples changes between datasets but the distribution of sample labels conditional on the samples remains unchanged between datasets, and other dataset-specific phenomenon and corrects them between any two datasets.

### BRIEF SUMMARY OF THE DISCLOSURE

In one aspect, a method for transferring a dataset-specific nature of a first dataset comprising sequencing results for a first plurality of specimen to a second dataset comprising sequencing results for a second plurality of specimen is disclosed. The method includes receiving, from a first entity, a first set of adaptation factors of the first dataset, wherein the first set of adaptation factors include two or more eigenvectors of the first dataset, and wherein the sequencing results for the first plurality of specimen cannot be reconstructed from the first set of adaptation factors without access to the first dataset. The method also includes generating a second set of adaptation factors of the second dataset, wherein the second set of adaptation factors include two or more eigenvectors of the second dataset, generating an adapted second dataset by adapting the dataset-specific nature of the second dataset to the dataset-specific nature of the second dataset based at least in part on the first set of adaptation factors and the second set of adaptation factors, and providing the adapted second dataset to the first entity.

In another aspect, a system for transferring a dataset-specific nature of a first dataset comprising sequencing results for a first plurality of specimen to a second dataset comprising sequencing results for a second plurality of specimen includes at least one memory and at least one processor coupled to the at least one memory. The system is configured to cause the at least one processor to execute instructions stored in the at least one memory to: receive, from a first entity, a first set of adaptation factors of the first dataset, wherein the first set of adaptation factors include two or more eigenvectors of the first dataset, and wherein the sequencing results for the first plurality of specimen cannot be reconstructed from the first set of adaptation factors without access to the first dataset; generate a second set of adaptation factors of the second dataset, wherein the second set of adaptation factors include two or more eigenvectors of the second dataset; generate an adapted second dataset by adapting the dataset-specific nature of the second dataset to the dataset-specific nature of the second dataset based at least in part on the first set of adaptation factors and the second set of adaptation factors; and provide the adapted second dataset to the first entity.

In still another aspect, a computer program product system for transferring a dataset-specific nature of a first dataset comprising sequencing results for a first plurality of specimen to a second dataset comprising sequencing results for a second plurality of specimen includes instructions stored on a non-transitory computer readable medium. The instructions, when executed by at least one processor on a computer, cause the processor to: receive, from a first entity, a first set of adaptation factors of the first dataset, wherein the first set of adaptation factors include two or more eigenvectors of the first dataset, and wherein the sequencing results for the first plurality of specimen cannot be reconstructed from the first set of adaptation factors without access to the first dataset; generate a second set of adaptation factors of the second dataset, wherein the second set of adaptation factors include two or more eigenvectors of the second dataset; generate an adapted second dataset by adapting the dataset-specific nature of the second dataset to the dataset-specific nature of the second dataset based at least in part on the first set of adaptation factors and the second set of adaptation factors; and provide the adapted second dataset to the first entity

In yet another aspect, a method includes receiving, from an entity, a first meta-specimen comprising a plurality of first meta-genes, wherein the first meta-specimen is associated with a first dataset of the entity; generating, from a second dataset, a second meta-specimen comprising a plurality of second meta-genes; adapting the second dataset to express a bias of the first dataset based at least in part on the plurality of first meta-genes and the plurality of second meta-genes; and providing the adapted second dataset to the entity. The method also may include labeling, via an artificial intelligence engine trained on the first dataset, one or more specimens of the second dataset based at least in part on the adapted second dataset.

### BRIEF DESCRIPTION OF THE DRAWINGS

FIG. 1 is a simplified general overview of a source dataset and a target dataset for use with the present disclosure;

FIG. 2A depicts a process of identifying dataset specific phenomenon by performing PCA (Principal Component Analysis) and comparing the resulting eigenvectors of the sample covariance, or PCA factors, of the source and target datasets, respectively;

FIG. 2B illustrates a process of correcting the dataset specific phenomenon between a source and target dataset;

FIG. 3 illustrates an adaptation pipeline that receives source and target molecular datasets and adapts the target datasets to the source dataset to obtain one or more adapted datasets having similar basis as the source dataset;

FIG. 4 illustrates a first adaptation pipeline 410 that receives a source dataset and generates adaptation factors to adapt any other dataset to the basis of the source dataset;

FIG. 5 is a flowchart of an embodiment of exemplary methods that may be performed by a spin adaptation engine when a single entity has access to both source and target datasets;

FIG. 6 illustrates a series of activities by and between two entities who wish to keep their patient datasets confidential;

FIG. 7A illustrates spin adaptation normalization of breast cancer RNA-Seq samples from two sources;

FIG. 7B illustrates prediction results based on the spin adaptation normalization of FIG. 7A;

FIG. 8A depicts the validation of homogenization of two datasets, along with a depiction of a ratio of source test samples with paired target samples to the k nearest neighbors of each test target sample in the test source dataset;

FIG. 8B illustrates hierarchical clustering of corrected test source and target samples from FIG. 8A;

FIG. 9 depicts results of an exemplary spin adaptation pipeline according to one aspect of the disclosure;

FIG. 10 illustrates survival curves for low-risk and high-risk groups identified in a plurality of cohorts, from a model which was trained on a different cohort, according to one aspect of the disclosure;

FIG. 11A depicts a privacy-preserving transfer of molecular models between a target lab and a source lab;

FIG. 11B depicts a regularized linear transformation between data factors of source and target, comprised of the PCA basis, gene-wise means, and gene-wise standard deviations of source and target, respectively;

FIG. 11C depicts the application of a learned transformation to a held-out prospective source dataset for correction, followed by application of a target-trained classifier on the corrected source dataset;

FIG. 12A depicts an alignment of paired expression datasets in a gene-expression space;

FIG. 12B depicts an unadapted pairing of four cancer subtypes in a two-dimensional space with UMAP;

FIG. 12C depicts a corrected pairing of the cancer subtypes of FIG. 12B in a two-dimensional space with UMAP according to the present disclosure;

FIG. 13A depicts scatter plots of a target basis and uncorrected source basis as compared to the target basis and a corrected source basis according to the present disclosure;

FIG. 13B depicts scatter plots of a target embedding and uncorrected source embedding as compared to the target embedding and a corrected source embedding according to the present disclosure;

FIG. 13C depicts scatter plots of paired expression values in the target and various sources;

FIG. 13D illustrates performance of 500 randomly generated sparse linear models evaluated on a simulated target and corrected source across varying sparsity levels;

FIG. 14A depicts a process for a target classifier to generate predictions on a corrected dataset;

FIG. 14B depicts a process for a target classifier to generate predictions on a second corrected dataset;

FIG. 15A depicts a comparison of the present method vs. several other correction methods for multiple breast cancer subtypes;

FIG. 15B depicts a comparison of the present method vs. several other correction methods for multiple colorectal cancer subtypes;

FIG. 15C depicts a comparison of the present method vs. several other correction methods for multiple pancreatic cancer subtypes;

FIG. 15D depicts a comparison of the present method vs. several other correction methods for multiple bladder cancer subtypes;

FIG. 16 illustrates subtype prediction performance on held-out source data;

FIG. 17 is a depiction of average silhouette score for multiple samples;

FIG. 18 illustrates quantification of dataset integration performance using batch LISI (bLISI) and tissue LISI (tLISI) metrics for multiple correction methods;

FIG. 19 is a distribution of log-partial hazards for a target-trained Cox model on a target dataset, source dataset, and corrected source for a pancreatic cancer type;

FIG. 20 is a survival curve for predicted low-risk and high-risk groups on the validation dataset for a pancreatic cancer type using Seurat;

FIG. 21 is a survival curve for predicted low-risk and high-risk groups on the validation dataset for a pancreatic cancer type using ComBat;

FIG. 22 is a distribution of log-partial hazards for a target-trained Cox model on a target dataset, source dataset, and corrected source for a colorectal cancer type;

FIG. 23 is a survival curve for predicted low-risk and high-risk groups on the validation dataset for a colorectal cancer type using Seurat;

FIG. 24 is a survival curve for predicted low-risk and high-risk groups on the validation dataset for a colorectal cancer type using ComBat;

FIG. 25 is a distribution of log-partial hazards for a target-trained Cox model on a target dataset, source dataset, and corrected source for a breast cancer type;

FIG. 26 is a survival curve for predicted low-risk and high-risk groups on the validation dataset for a breast cancer type using Seurat;

FIG. 27 is a survival curve for predicted low-risk and high-risk groups on the validation dataset for a breast cancer type using ComBat;

FIG. 28 is a distribution of log-partial hazards for a target-trained Cox model on a target dataset, source dataset, and corrected source for a pancreatic cancer type;

FIG. 29 is a survival curve for predicted low-risk and high-risk groups on the validation dataset for a pancreatic cancer type before correction;

FIG. 30 is a survival curve for predicted low-risk and high-risk groups on the validation dataset for a pancreatic cancer type using SpinAdapt;

FIG. 31 is a distribution of log-partial hazards for a target-trained Cox model on a target dataset, source dataset, and corrected source for a colorectal cancer type;

FIG. 32 is a survival curve for predicted low-risk and high-risk groups on the validation dataset for a colorectal cancer type before correction;

FIG. 33 is a survival curve for predicted low-risk and high-risk groups on the validation dataset for a colorectal cancer type using SpinAdapt;

FIG. 34 is a distribution of log-partial hazards for a target-trained Cox model on a target dataset, source dataset, and corrected source for a breast cancer type;

FIG. 35 is a survival curve for predicted low-risk and high-risk groups on the validation dataset for a breast cancer type before correction;

FIG. 36 is a survival curve for predicted low-risk and high-risk groups on the validation dataset for a breast cancer type using SpinAdapt;

FIG. 37 includes UMAP plots for dataset integration, labeling samples by dataset;

FIG. 38 includes UMAP plots for dataset integration, labeling samples by cancer subtype; and

FIG, 39 is an illustration of a block diagram of an implementation of a computer system in which some implementations of the disclosure may operate.

### DETAILED DESCRIPTION

This application is related to U.S. Patent Publication No. 2021/0098078, published Apr. 1, 2021, and titled “Systems And Methods For Detecting Microsatellite Instability Of A Cancer In A Liquid Biopsy Assay,” and U.S. Patent Publication No. 2021/0057042, published Feb. 25, 2021, and titled “Systems And Methods For Detecting Cellular Pathway Dysregulation In Cancer Specimens,” the contents of which are both incorporated herein by reference in their entireties for all purposes.

The terms and phrases “nature or biases, domain shifts, covariate shifts, or other dataset specific phenomenon” are defined to be a difference in the probability distribution of one or more features within a dataset with respect to another dataset where a data-specific nature, bias, shift, or phenomenon may refer to the respective nature, bias, shift, or phenomenon of that specific data.

In one aspect, the present disclosure describes a method, a system, and a computer program product that enable the transfer of molecular predictors between laboratories without disclosure of the sample-level training data for the predictor, thereby allowing laboratories to maintain ownership of the training data and protect patient privacy. The present approach is based on matrix masks from privacy literature, where the sample-level data and matrix mask is kept private, while the output of the matrix mask is shared publicly.

The present disclosure includes an unsupervised genomic correction algorithm, e.g., an RNA correction algorithm, that enables the transfer of molecular models without requiring access to patient-level data. It computes data corrections only via aggregate statistics of each dataset, thereby maintaining patient data privacy. Furthermore, decoupling the present method from its training data allows the correction of new prospective samples, enabling evaluation on validation cohorts. Despite an inherent tradeoff between privacy and performance, the present outperforms current correction methods that require patient-level data access.

In another aspect, the present disclosure relates to a system, method, and computer program product for the transfer and validation of diagnostic models across transcriptomic datasets. The common task of integration (homogenization) across multiple transcriptomic datasets is also evaluated for multiple cancer types, comparing various integration methods. The disclosure also demonstrates the application of the present method in transfer of prognostic models across multiple cancer types. That method outperforms other batch correction methods in the majority of these diagnostic, integration, and prognostic tasks, without requiring direct access to sample-level data. Therefore, the present disclosure may also be preferable for transferring molecular predictors across datasets when training dataset is available and data privacy is not an issue.

Example Purposes and Use Cases

A spin adaptation engine may be used to correct biases between RNA-Seq and/or microarray datasets across different platforms and sequencing libraries. A spin adaptation engine may allow for the integration of a plurality of molecular datasets into another molecular dataset, enabling the increase of the overall molecular dataset.

A spin adaptation engine may be used to generate a principal component profile on a first molecular dataset, which may be applied, in combination with the spin adaptation engine, on a second molecular dataset. As a result, the second molecular dataset may be adapted to the first molecular dataset without requiring the transfer of either molecular dataset from one owner to another.

A spin adaptation engine may be used to homogenize a first molecular dataset with a second molecular dataset, where the first and second molecular datasets are generated from different laboratory technologies. For example, the first molecular dataset may be a source microarray dataset and the second molecular dataset may be a target assay dataset, such as a target RNA-seq dataset.

A spin adaptation engine may be used to homogenize a first molecular dataset with a second molecular dataset, or to “spin” from the first dataset to the second dataset, where the first and second molecular datasets are generated from different laboratories. For example, the first molecular dataset may be a source germline dataset from a laboratory in a first location and the second molecular dataset may be a source germ line dataset from a laboratory in a second location.

A spin adaptation engine may be used to validate an artificial intelligence engine, such as an implementation of a machine learning algorithm trained from a first molecular dataset, to ensure that the resulting model is not overfitted to any biases in the first molecular dataset.

A spin adaptation engine may be employed to enable the transfer of molecular classifiers across labs without the need of either laboratory to change its operational or lab pipelines or share individual-level patient molecular data information.

A spin adaptation engine may be employed across a number of different molecular datasets in a pairwise fashion, each dataset with a first dataset to improve the robustness of an artificial intelligence engine trained on the number of different molecular datasets at the same time.

A spin adaptation engine may be employed according to any of the above on datasets other than molecular datasets. In one example, an exemplary dataset may include imaging data, such as an image of an x-ray, ct-scan, mri, pathology slide, or other image. In another example, an exemplary dataset may include clinical information, such as a cohort of patients in an electronic medical record database or electronic health record database.

An artificial intelligence engine may include a trained model, such as a machine-learning algorithm (MLA) or a neural network (NN) may be trained from a training data set such as a plurality of matrices having a feature vector for each subject. In an exemplary prediction or classification profile, a training data set may include imaging, pathology, clinical, and/or molecular reports and details of a subject, such as those curated from an EHR or genetic sequencing reports. MLAs include supervised algorithms (such as algorithms where the features/classifications in the data set are annotated) using linear regression, logistic regression, decision trees, classification and regression trees, Naïve Bayes, nearest neighbor clustering; unsupervised algorithms (such as algorithms where no features/classification in the data set are annotated) using Apriori, means clustering, principal component analysis, random forest, adaptive boosting; and semi-supervised algorithms (such as algorithms where an incomplete number of features/classifications in the data set are annotated) using generative approach (such as a mixture of Gaussian distributions, mixture of multinomial distributions, hidden Markov models), low density separation, graph-based approaches (such as mincut, harmonic function, manifold regularization), heuristic approaches, or support vector machines. NNs include conditional random fields, convolutional neural networks, attention based neural networks, deep learning, long short term memory networks, or other neural models where the training data set includes a plurality of tumor samples, RNA expression data, DNA mutation data, medical data, clinical data, imaging data, or other types of relevant data for each sample, and pathology reports covering imaging data for each sample. While MLA and neural networks identify distinct approaches to machine learning, the terms may be used interchangeably herein. Thus, a mention of MLA may include a corresponding NN or a mention of NN may include a corresponding MLA unless explicitly stated otherwise and an artificial intelligence engine may include one or more of either.

In one embodiment, the examples provided herein may operate in accordance with receiving source and target datasets, their corresponding basis, or a combination thereof, and the distribution of samples in the target dataset may be changed with respect to the distribution of samples in the source dataset while leaving conditional sample label distribution (i.e., the labels used and/or predicted during classification from an artificial intelligence engine) unchanged across both the source and target datasets.

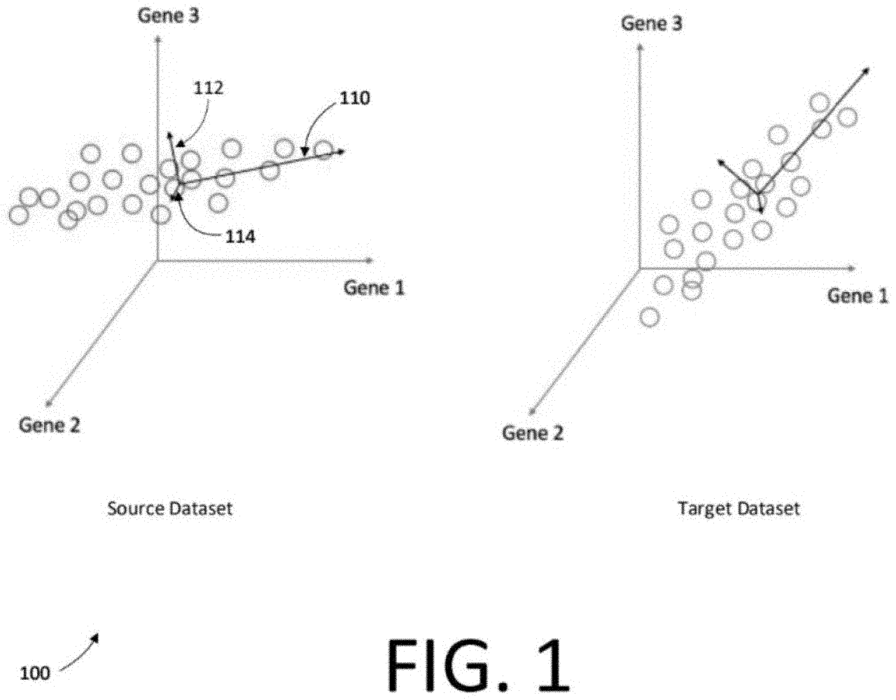

FIG. 1 illustrates a simplified general overview of a source dataset and a target dataset, each having a difference in expression due to nature or biases, domain shifts, covariate shifts, or other dataset specific phenomenon. Each circle represents a molecular data set from a single individual such as a patient. The 3-dimensional orthogonal arrows represent a simplified PCA basis. In other embodiments, the basis may be represented by other orthogonal basis or even non-orthogonal basis such as those generated by sparse PCA, NMF, or autoencoders. While patients are generally sequenced to access up to 20,000 genes or 140,000 transcripts, FIG. 1 has been simplified to only three genes for illustrative purposes.

In a three gene dataset, where each axis of a three-dimensional plot represents an abundance of reads for a genetic assay such as the RNA expression of that gene and a patient is plotted with respect to their sequencing results according to the measured abundance at that gene. PCA may then be performed on the dataset to identify dataset specific phenomenon. In some examples a N-dimensional PCA analysis may be performed, where FIG. 1 illustrates a three-dimensional PCA analysis. The eigenvectors 110, 112, and 114 which originate from the source dataset illustrate the results of the PCA analysis. The PCA basis can be interpreted as representation of various molecular subtypes in the dataset, and may be referred to as meta-patients when the source dataset comprises patient molecular data or may be referred to as meta-specimen when the source dataset comprises specimen molecular data. Meta-patient, meta-specimen, and meta-genes may be used interchangeably when each specimen is associated with a patient and each gene of a plurality of genes are associated with the sequencing results of a specimen. A surprising use of the techniques herein is that performing the first variance measurement/characterization encoding and ranking the features of the dataset according to their variance without performing the culling stop of the dimensionality reduction allows one to utilize the PCA in this manner to identify correlation between the variable genes 1, 2, and 3 in each dataset and sets the algorithm up for transforming from one dataset to the other. In various embodiments, the datasets will represent dimensionalities in the tens or hundreds of thousands of variables and the reduced dimensions will result in eigenvectors representing the directions, or principal components, of maximum variance in the data while retaining the most useful information. An additional step, such as dropping the eigenvectors with the smallest magnitudes, may be implemented to complete the dimensionality reduction. In some examples, none of the eigenvectors may be dropped, for example when the dataset has high variance across all features. In another example the lowest 25% of the eigenvectors may be dropped. In another example, models may be trained using a random forest which predicts model outcomes from the differing number of dropped eigenvectors and the number of eigenvectors to drop may be identified as the most eigenvectors without dropping the model performance below an accuracy threshold such as 95%, 90%, or 85% accuracy. With respect to FIG. 1, dropping eigenvector 114 would effectively reduce the dimensionality from three variables to two variables with the least loss of information, thereby projecting the three-dimensional dataset into a smaller dimensional subspace. Eigenvector 110 represents the projection of gene 1 into the new subspace which maximizes variance of the dataset. Correspondingly, eigenvector 112 represents gene 3 and eigenvector 114 represents gene 2. Presuming that the PCA will eventually drop eigenvector 114, the remaining eigenvectors, 110 and 112, will become PCA 1 and PCA 2. The new subspace will be slightly rotated and may have a different average mean. In another example, dataspaces may be zero mean normalized to remove a need to perform an average means shift.

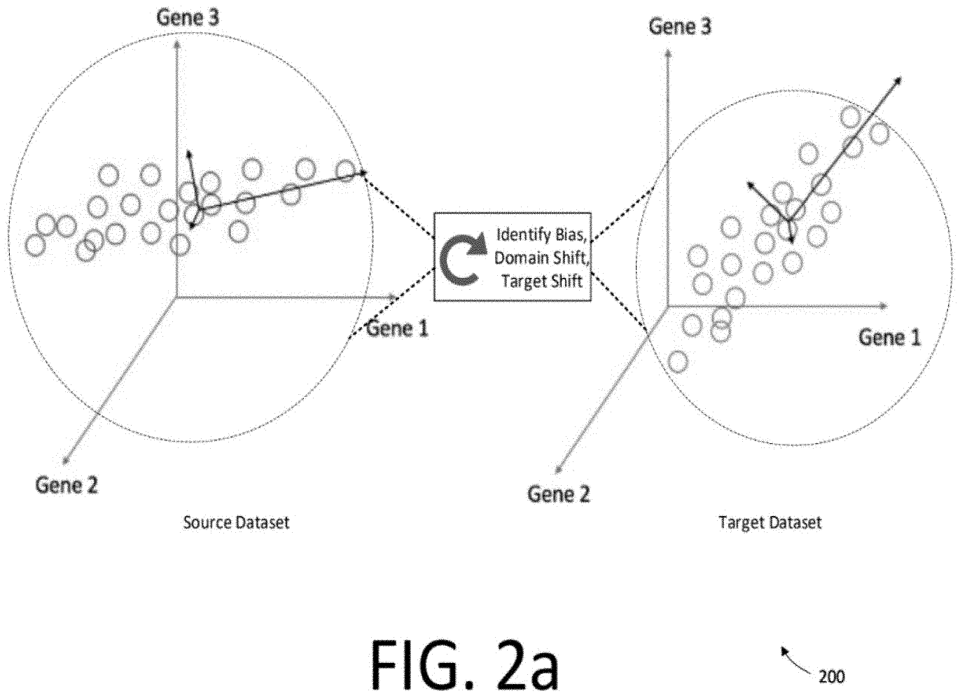

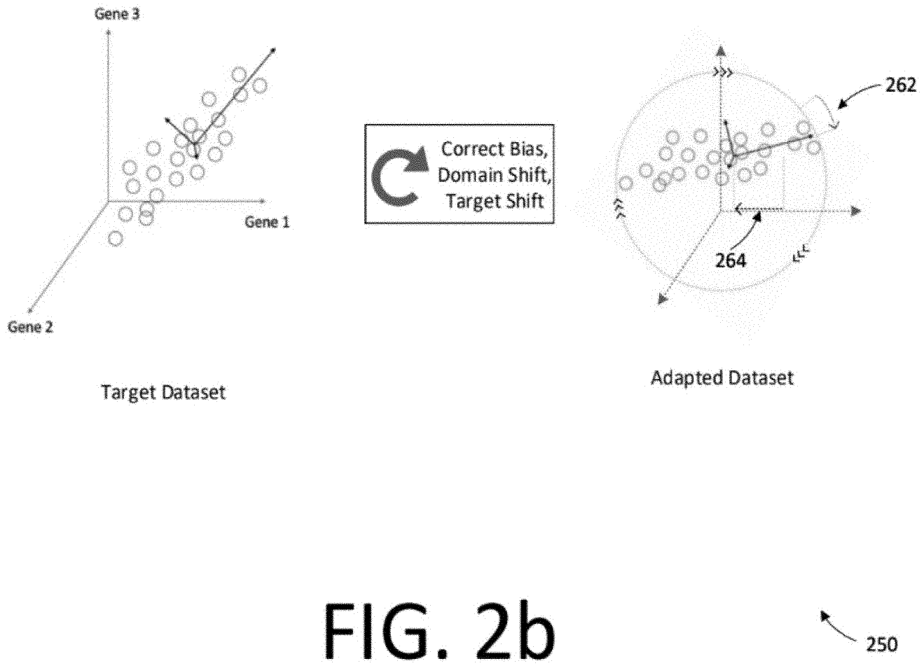

FIG. 2A illustrates the process of identifying the dataset specific phenomenon such as bias, domain shifts, or target shifts by performing PCA and comparing the resulting eigenvectors of the sample covariance, or PCA factors, of the source and target datasets, respectively.

Performing the same PCA on the target dataset will result in another collection of eigenvectors from the target sample covariance matrix. While the eigenvectors are displayed with only a mean shift and a rotation here, each new dataset will provide eigenvectors with different magnitudes than illustrated. It is important to note that PCA is orthogonal, so even if there are differing magnitudes, their angles will always be 90 degrees whereas sparse PCA, autoencoders, SVD, and NMF may have angles other than 90 degrees, too, making them non-orthogonal. After dropping the weakest eigenvectors, the target dataset may have its own, unique set of PCA factors.

FIG. 2B illustrates the process of correcting the dataset specific phenomenon between a source and target dataset. Correction may be performed by identifying a rotation and an average mean difference between the corresponding PCA factors of the source and target datasets. Once identified the target dataset may be corrected by rotating to offset the rotational difference and may be shifted along the substance axis to the corrected position. Rotational correction 262 may be applied to spin the PCA basis from the dataset specific phenomenon of the target dataset to the source dataset and average mean correction 264 may be applied to similarly shift the PCA basis. In some embodiments, the datasets are zero-mean shifted as a stage of preprocessing which renders the average mean correction unnecessary. While the basis illustrated are orthogonal, the present embodiments may also convert non-orthogonal datasets, too, without departing from the scope of the disclosure,

Spin Adaptation Pipeline:

In some embodiments, a first entity, such as a laboratory with a source dataset may employ an artificial intelligence engine on the source dataset and desire to validate the results with a target dataset such as a public dataset or to incorporate another dataset into the training of the model such as a dataset from another entity.

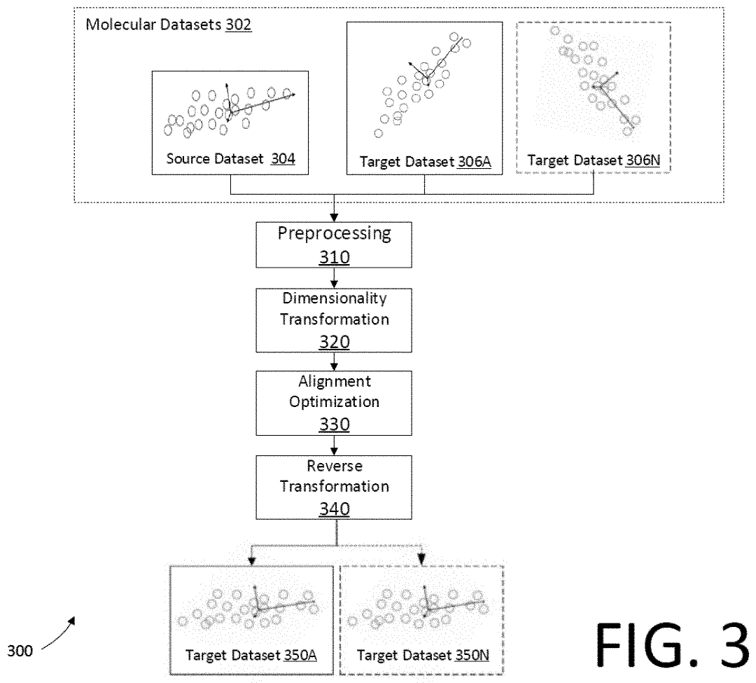

FIG. 3 illustrates an adaptation pipeline 300 which receives molecular datasets 302, such as the source dataset 304 and one or more target datasets 306A-N and adapts the one or more target datasets 306A-N to the source dataset 304 to obtain one or more adapted representations/datasets 350A-N having similar basis as the source dataset.

Embodiments of a spin adaptation engine, or adaption pipeline, 300 may include receiving two or more molecular datasets 302 such as a source dataset 304 and one or more target datasets 306A-N. In one embodiment, molecular datasets 302 may include assay data such as next-generation sequencing (NGS) data, DNA sequencing data, RNA sequencing data, methylation analysis data, copy number data, or other molecular based metrics for a plurality of subjects. A source dataset 304 may include the internal dataset curated by a first entity such as a laboratory, clinic, university, hospital, pharmaceutical company, or another entity. In some examples, the first entity may acquire one or more target datasets 306A-N in order to supplement an existing dataset, such as the source dataset 304. In some examples, the first entity may have trained an artificial intelligence engine on an existing dataset and desire to validate the performance of the artificial intelligence engine on the one or more target datasets. In some examples, the first entity may provide a trained artificial intelligence engine to another entity having the one or more target datasets 306A-N, for the other entity to use the trained artificial intelligence engine on the one or more target datasets 306A-N. The molecular datasets 302 may be provided to an adaptation pipeline 300 which identifies the nature or biases, domain shifts, covariate shifts, or other dataset specific phenomenon and corrects them between the molecular datasets 302. The output of the adaptation pipeline 300, the adapted dataset(s) 350A-N, may be separately (and/or concurrently) generated based on a mode of operation of the adaptation pipeline. In one example, the adaptation pipeline 300 may operate as a batch correction algorithm, which learns adaptation factors and corrects each of the one or more target datasets to the source dataset but maintains the dataset individuality, and thus does not require combining each into a single adapted dataset having a combined size of all of the target datasets accumulated together. In a second mode of operation, the one or more target datasets 350A-N may be integrated into a single adapted dataset, with or without the source dataset as they have all been corrected to having the same basis representation once processed through the adaptation pipeline 300.

The received datasets may be preprocessed at a preprocessing stage 310 to identify the nature or biases, domain shifts, covariate shifts, or other dataset specific phenomenon, of the molecular data included, the system may be configured for only RNA or DNA molecular datasets, or the uploader of the datasets may flag the system to identify the dataset uploaded. Analysis may include observation of the number of data points for each subject, the type of information associated with each data point, or other identifying information to determine whether the uploaded data includes RNA or DNA molecular information. For example, molecular information having 20,000+ data points may be RNA data. Molecular information having a data point assigned to a value of 0 or 1 may be DNA data while having a data point assigned to a value 0-100000, or normalized as real valued, may be RNA data. Preprocessing may further include steps or stages for zero-mean scaling, normalization, sorting, and/or feature vector correction to ensure both datasets have the same number of features. Preprocessing is disclosed in more detail, below.

After preprocessing, the two or more molecular datasets 302 may be encoded at a dimensionality transformation stage 320 to find basis representation for each dataset. Encoding may include a transformation such as a dimensionality transformation. The selected transformation may be reversible and include a subsequent decoding transformation. In one aspect, encoding may also serve to identify, or capture, the nature or biases, domain shifts, covariate shifts, or other dataset specific phenomenon, of the molecular dataset. Dimensionality transformation is disclosed in more detail, below.

The encodings (basis representation) of each dataset generated from stage 320 may be sent to an alignment optimization stage 330 for alignment optimization to identify a correction transformation which may be applied to adapt the one or more target datasets 306A-N to the nature of the source dataset 304. In this manner, a plurality of eigenvectors of the covariance matrix for each dataset are generated, these eigenvectors serve as the PCA Factors for the alignment, whereas the sample encodings on these eigenvectors serve as the PCA Embeddings. Although the alignment optimization procedure is performed between PCA factors of the datasets, the subsequent correction transformation is employed to transform the target PCA Embeddings to the basis of the source dataset. The correction transformation may be applied to the PCA Embeddings of one or more target datasets to generate one or more nature corrected datasets which are generated as an output for stage 330. Alignment optimization is disclosed in more detail, below.

The nature corrected dataset output from stage 330 may then be sent to a reverse transformation stage 340 for reverse transformation, or decoding, into an adapted dataset, such as one of target datasets 350A-N. In one example, the adapted dataset may be joined with the source dataset and applied to train an artificial intelligence engine with both the data of the source and one or more (adapted) target datasets 350A-N. In another example, a model trained on the source dataset may be evaluated on the (adapted) target dataset to validate that the model trained on the source dataset provides high accuracy metrics on the target dataset when evaluated and is thus accurate. In yet another example, the trained model may simply be provided the adapted dataset to generate predictions or classifications for the samples/subjects in the target dataset. The target datasets 350A-N are output from stage 340. Reverse Transformation is disclosed in more detail, below.

In one embodiment, the above stages may be repeated for each additional target dataset to join all target datasets into one joint adapted dataset.

In some instances, a first entity may build a robust artificial intelligence engine trained on the source dataset and provide the artificial intelligence engine to a second entity, such as a university, hospital, or pharmaceutical company. The model trained on the source data may be shared between the first entity and the second entity, but neither the source dataset nor the target dataset is shared between the entities. In such a circumstance, the first entity may use the adaptation pipeline to generate adaptation factors which may be supplied to the second entity along with an adaptation pipeline for the second entity to generate the adapted dataset on their own.

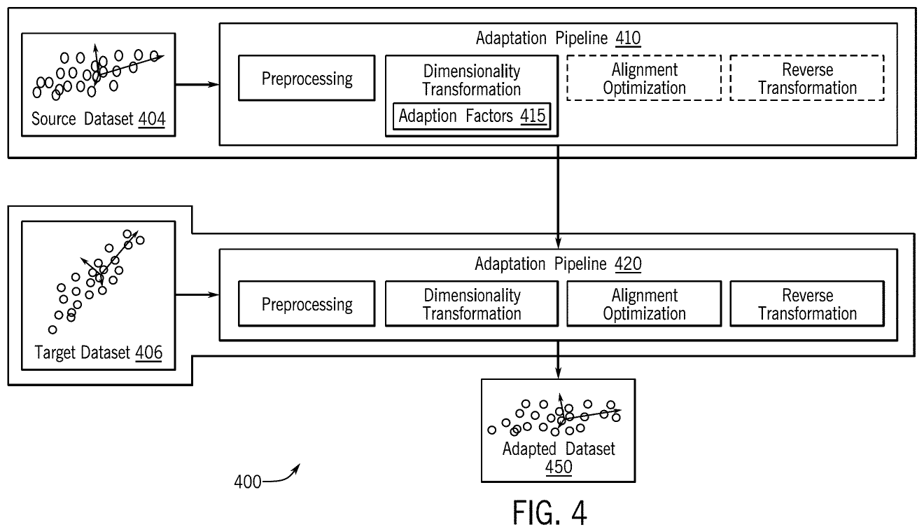

FIG. 4 illustrates a first adaptation pipeline 410 which receives the source dataset 404 and generates adaptation factors 415 which may be used to adapt any other dataset to the basis of the source dataset. A second adaptation pipeline 420 may receive the adaptation factors 415 of the source dataset and the target dataset 406 to adapt the target dataset to the nature of the source dataset.

The first adaptation pipeline 410 may operate according to the adaptation pipeline 300 to perform preprocessing and dimensionality transformation stages on the source dataset but not operate the alignment optimization or reverse transformation stages as these stages require, at least in part, the adaptation factors of both the source and target datasets which are not shared across entities.

Adaptation factors 415 may be generated from the dimensionality transformation and output from the pipeline to a user or automatically passed to a second adaptation pipeline 420 via a communication protocol, the World Wide Web, or other communication mediums. Adaptation factors are disclosed in more detail, below.

The second adaptation pipeline 420 may operate according to the adaptation pipeline 300 to perform preprocessing and dimensionality transformation stages on the target dataset, receive the adaptation factors 415 generated from the source dataset, and perform the alignment optimization and reverse transformation stages to generate the adapted dataset 450.

In certain aspects, the spin adaptation engine 400 calculates independently a transformation basis of a source dataset and a target dataset; finds a low rank affine transformation between the source dataset basis and the target dataset basis; and maps new data through the basis transformation.

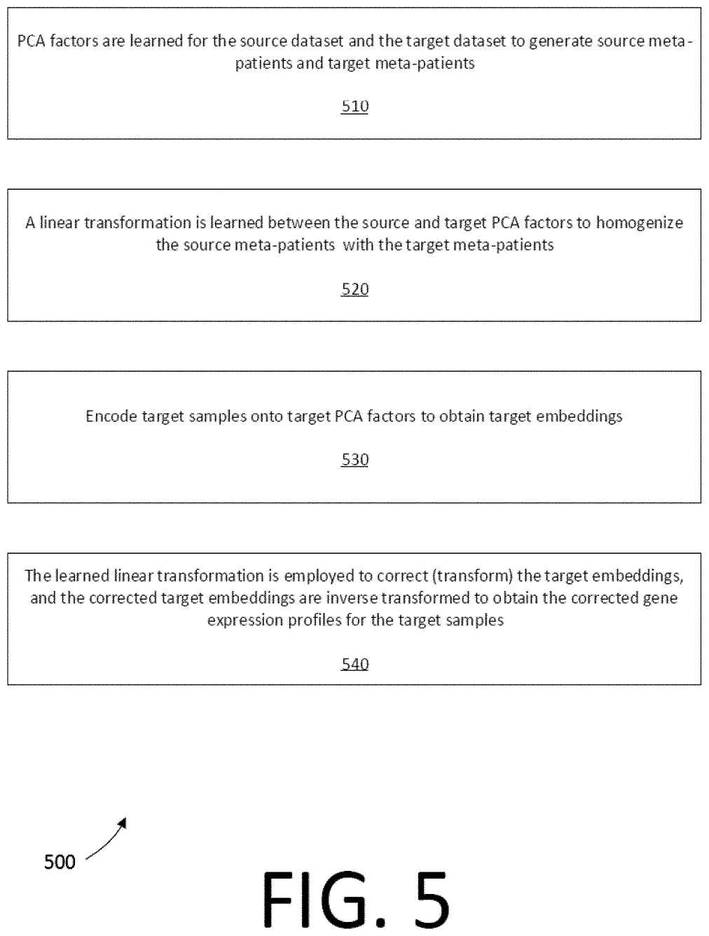

FIG. 5 illustrates a flowchart of an embodiment of exemplary methods that may be performed by a spin adaptation engine when a single entity has access to both the source and the target datasets.

In 510, PCA factors are learned for the source dataset and the target dataset, independently, to generate source meta-patients and target meta-patients, respectively. In one example, the source dataset may be projected onto a learned subspace representation to obtain subspace embeddings for the high-dimensional RNA-Seq (or microarray) source dataset.

In 520, a linear transformation is learned between the source and target PCA factors to homogenize the source meta-patients with the target meta-patients.

In 530, the spin adaptation engine may encode target samples onto target PCA factors to obtain target embeddings. the corrected source embeddings are used to recover the gene-expression profiles of the source samples in the gene-expression target space.

In 540, the learned linear transformation is employed to correct (transform) the target embeddings, and the corrected target embeddings are inverse transformed to obtain the corrected gene expression profiles for the target samples.

The above corrections are invertible and performed in a latent space, thereby, evading the need for pair-wise matching of similar patients between the two datasets. Although the corrections are performed in a latent space, the output of the adaptation engine is high-dimensional gene-expression profiles, made possible because of the invertibility of the encoding transformation.

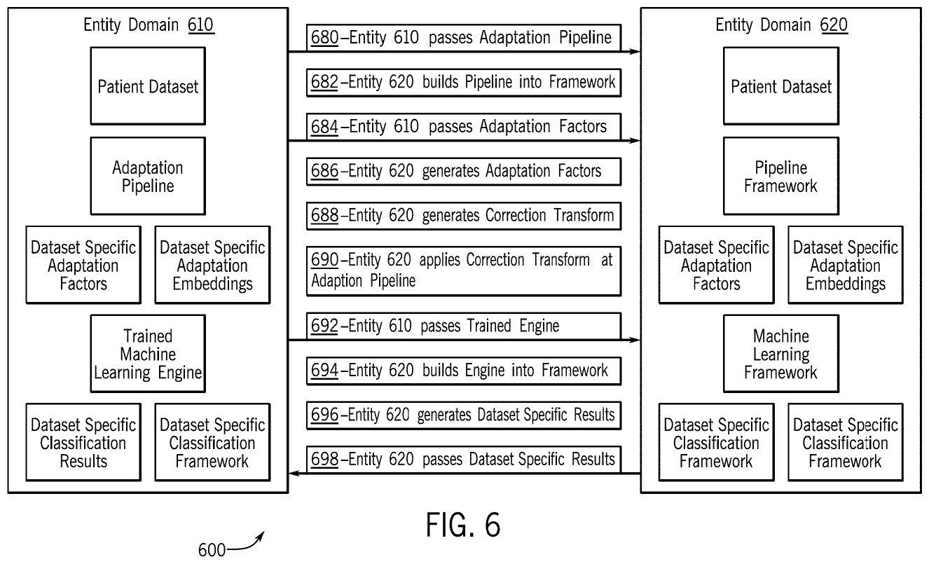

FIG. 6 illustrates a series of activities by and between two entities who wish to keep their patient datasets confidential.

A first entity maintains an entity domain 610 with a patient dataset, an adaptation pipeline configured in accordance with FIG. 3, dataset specific factors and embeddings for the patient dataset, a trained machine learning engine, dataset specific classification results from the engine, and a dataset specific classification framework for storing additional dataset specific classification results.

A second entity maintains an entity domain 620 with a patient dataset, an adaptation framework which may implement an adaptation pipeline configured in accordance with FIG. 3, dataset specific factors and embeddings for the patient dataset which may be generated from the adaptation pipeline once configured, a machine learning framework which may implement a trained machine learning engine, and one or more dataset specific classification frameworks for storing dataset specific classification results from the engine.

The first and second entity may collaborate to generate machine learning classification results for the second entity's patient dataset without ever sharing the patient dataset between either entity.

In a first transmission at step 680, the first entity may share the adaptation pipeline with the second entity. At step 682, the second entity may utilize their pipeline framework to build the adaptation pipeline within their domain. In a first embodiment, this may include installing an access portal which accesses an API to the first entity's adaptation pipeline. In a second embodiment, this may include installing a software package which communicates with the first entity's adaptation pipeline for only a subset of the calculations needed to generate adaptation factors without completely transmitting the patient dataset. In a third embodiment, this may include transmission of a library which may instantiate an independent adaptation pipeline without the first entity's oversight.

In a second transmission at step 684, the first entity may share adaptation factors from their patient dataset without sharing the corresponding adaptation embeddings, thus avoiding the second entity from accessing the first entity's patient dataset. At step 686, which may operate after the adaptation framework is accessed or concurrently with step 684, the second entity generates their own adaptation factors from their patient dataset. At step 688, the second entity may generate the correction transform from the first entity's adaptation factors and their own adaptation factors. At step 690, the second entity may correct their patient dataset with the correction transform to generate an adapted dataset. At step 692, the first entity may provide, or pass, the trained machine learning engine. As with the adaptation pipeline, this may be provided through access to an API, a software pipeline that communicates with the first entity's trained machine learning engine, or a library which operates entirely within the second entity's domain. At step 694, the second entity builds the machine learning engine in their machine learning framework. At step 696, the second entity generates dataset specific results using the trained machine learning engine of the first entity and their adapted patient dataset. At step 698, the second entity may share their dataset specific classification results with the first entity for the purposes of improving the trained machine learning model, validating the model's accuracy, conducting research, improving patient sequencing results, or improving corresponding diagnosis or treatment.

In another embodiment, multiple sharing entities may be willing to collaborate with a single receiving entity provided maintenance of their data security. The receiving entity may generate the basis factors according to the methods described herein and supply each of the sharing entities with the basis factors. In one example, there may be three sharing entities, such as a laboratory, hospital or institution, and a pharmaceutical company, each receiving the basis factors generated by the receiving entity. Each sharing entity may then generate their own basis factors from their private datasets, supply their own basis factors and the received basis factors to a pipeline, and generate adapted datasets which may be shared with the receiving entity without compromising their data security. In one example, a generated dataset may be provided as a transformed embedding (such as PCA embedding or another transformation) only.

Inversely, in another embodiment, multiple sharing entities may be willing to collaborate with a single receiving entity provided maintenance of their data security. Each of the multiple sharing entities may calculate its own basis factors and share them with the receiving entity. The receiving entity may then provide the received basis factors into an adaptation pipeline as described herein and generate a correction transform for each of the sharing entities. The respective correction transform may be shared with each sharing entity to enable them to correct and encode their private data for sharing with the receiving entity.

In another embodiment, an adapted dataset may be generated between a source and target dataset, however, only the embeddings associated with a cohort of patients may be shared from the target dataset instead of the embeddings from the entire target dataset. A cohort may be identified as any subset of the dataset where each patient includes specified features such as features selected from their genomic information, clinical information, or other patient information. In one example, a cohort may include a breast cancer cohort having stage III diagnosis and receiving a specified treatment such as a combination of localized radiation and chemotherapy. Another cohort may be selected based on FGFR2, MAP3K1, TNRC9, BRCA1, or BRCA2. Additional cohort selection criteria are identified in U.S. patent application Ser. No. 16/771,451, filed Jun. 10, 2020 and titled “Data Based Cancer Research And Treatment Systems And Methods,” which is incorporated herein by reference for all purposes,

In another embodiment, specific cohorts from a plurality of target datasets may be generated and shared when the number of qualified dataset entities is small between all available sources. For example, a cohort including a rare cancer subtype such as adenosquamous carcinoma of the lung, a hybrid of adenocarcinoma and squamous cell lung cancer, large cell neuroendocrine carcinoma, or an aggressive subtype of non-small cell lung cancer. Cohorts for rare cancer subtypes may include only a few patients (tens or hundreds) even in large datasets (thousands or millions of patients),In such an example, a first target dataset may provide only seven patients, a second target dataset may provide only twenty patients, a third dataset may provide only eleven patients, and a fourth dataset may provide only thirty-six patients. While subsequent analysis on any one dataset may be insufficient for clinical purposes, analysis on each of the datasets adapted to a source basis may provide enough patients to arrive at a clinically validated assessment.

Adaptation Pipeline for RNA Molecular Datasets

Selection of the normalization, sorting, and/or feature vector correction steps of preprocessing; transformations, encoders, and/or dimensionality reductions algorithms of the dimensionality transformation; optimization transformation and/or algorithms of the alignment optimization; and the corresponding reverse/inverse transformation and/or decoders of the reverse transformation to apply at each respective stage of the adaptation pipeline may be tied to the type of molecular data the datasets comprise.

For example, when the source and target datasets include RNA molecular data the pipeline may be configured as follows:

Preprocessing may include sorting each feature of the datasets to be in the same order, identifying feature mismatches between datasets and either removing the mismatched features from the dataset if those features are not important to the model accuracy, padding the missing features with null values if those features are not important to the model accuracy, predicting the mismatched features from the dataset if they are important, or ignoring mismatches for robust transformations which allow for feature mismatch; and/or normalizing the datasets with a variance stabilization transform to stabilize the variance of all features, which is a critical preprocessing step in many supervised and unsupervised machine learning algorithms which are sensitive to the variance of the features considered.

Dimensionality transformation may include selecting a PCA transformation or another dimensionality reduction algorithm, where the number of PCA basis is the minimum of the number of features and the number of patients in the dataset considered, and applying the PCA transformation to the datasets to identify PCA factors, principal component factors, the adaptation factors, or encoding basis and PCA embeddings or encoded values.

Alignment Optimization may include identifying a non-convex optimization formulation, a corresponding objective function to minimize, a relaxation process for the objective function such that the objective function is convex, In one example, the non-convex optimization may minimize the mean square error between the transformed target factors and the source factors with a matrix-rank penalization on the transformation between the source and target factors, and the relaxation may entail minimization of the mean square error between the transformed target factors and the source factors with a trace-norm penalization on the transformation between the source and target factors, which can be solved using a proximal gradient descent method, where the posed method iteratively (i) minimizes the Euclidean distance between the transformed source factors and target factors by moving in the direction of steepest descent and (ii) applies the proximal operator for the trace-norm of the transformation to the output of the previous descent step, till convergence of the iterative method. In another example, the non-convex optimization may minimize the mean square error between the transformed target factors and the source factors with a hard constraint on the matrix-rank of the transformation between the source and target factors, which can be solved using a projected gradient descent method, where the posed method iteratively (i) minimizes the Euclidean distance between the transformed source factors and target factors by moving in the direction of steepest descent and (ii) applies the projection operator for the low matrix-rank approximation of the transformation to the output of the previous descent step, till convergence of the iterative method. The output of the proximal gradient descent method or the projected gradient descent method.

Reverse transformation may include selecting an inverse PCA transformation or decoder and applying the inverse PCA transformation or decoder to the corrected embeddings of the target dataset to generate the adapted dataset.

In one example, a source dataset may comprise a plurality of patients having RNA Sequencing results, including expression for each of 140,000+ RNA transcripts in a patient by transcript matrix. A basis transformation may measure the variance across each of the 140,000+ RNA transcripts, such as a plurality of eigenvectors representing a meta-patient where each eigenvector represents a meta-gene/meta-transcript for the meta-patient. Eigenvectors falling below a threshold of variance may be removed from the dataset as an aspect of dimensionality reductions. In one example, eigenvectors below 0.10, 0.20, 0.30, 0.40, or 0.50 may be deleted. In another example, eigenvectors associated with transcripts which are not informative to a trained model may be excluded, such as transcripts receiving a lower weight or coefficient than other transcripts. A transformation may be learned between the basis factors of the meta-patients for each dataset and applied to each patient of the plurality of patients in the target dataset to generate the adapted dataset. The adapted dataset may be a matrix of patients by adapted transcripts or only the basis embeddings of the adapted transcripts for each patient.

Adaptation Pipeline for DNA Molecular Datasets

When the source and target datasets include assay data such as DNA molecular data the pipeline may be configured as follows:

Preprocessing may include sorting each feature of the datasets to be in the same order, identifying feature mismatches between datasets and either removing the mismatched features from the dataset if those features are not important to the model accuracy, padding the missing features with null values if those features are not important to the model accuracy, predicting the mismatched features from the dataset if they are important, or ignoring mismatches for robust transformations which allow for feature mismatch; and/or normalizing the datasets such that the features have unit-variance.

Dimensionality transformation may include selecting a sparse PCA transformation or another dimensionality reduction algorithm, where the number of sparse PCA basis is the minimum of the number of features and the number of patients in the dataset considered, and applying the sparse PCA transformation to the datasets to identify sparse PCA factors, principal component factors, the adaptation factors, or encoding basis and sparse PCA embeddings or encoded values.

Alignment Optimization may include identifying a non-convex optimization formulation, a corresponding objective function to minimize, a relaxation process for the objective function such that the objective function is convex. In one example, the non-convex optimization may minimize the mean square error between the transformed target factors and the source factors with a matrix-rank penalization on the transformation between the source and target factors, and the relaxation may entail minimization of the mean square error between the transformed target factors and the source factors with a trace-norm penalization on the transformation between the source and target factors, which can be solved using a proximal gradient descent method, where the posed method iteratively (i) minimizes the Euclidean distance between the transformed source factors and target factors by moving in the direction of steepest descent and (ii) applies the proximal operator for the trace-norm of the transformation to the output of the previous descent step, until convergence of the iterative method. In another example, the non-convex optimization may minimize the mean square error between the transformed target factors and the source factors with a hard constraint on the matrix-rank of the transformation between the source and target factors, which can be solved using a projected gradient descent method, where the posed method iteratively (i) minimizes the Euclidean distance between the transformed source factors and target factors by moving in the direction of steepest descent and (ii) applies the projection operator for the low matrix-rank approximation of the transformation to the output of the previous descent step, till convergence of the iterative method. The output of the proximal gradient descent method or the projected gradient descent method.

Reverse transformation may include selecting an inverse sparse PCA transformation or decoder and applying the inverse sparse PCA transformation or decoder to the corrected target dataset to generate the adapted dataset. In other examples, decoding or reverse transformations may be performed using inverse NMF, ICA, or other matrix factorizations.

In one example, a source dataset may comprise a plurality of patients having DNA Sequencing results, including identification of a presence of each variance of 20,000+ DNA genes in a patient by gene variant matrix. A basis transformation may measure the variance across each of the 20,000+ DNA gene variants, such as a plurality of eigenvectors representing a meta-patient where each eigenvector represents a meta-gene for the meta-patient. Eigenvectors falling below a threshold of variance may be removed from the dataset as an aspect of dimensionality reductions. In one example, eigenvectors below 0.10, 0.20, 0.30, 0.40, or 0.50 may be deleted. In another example, eigenvectors associated with gene variants which are not informative to a trained model may be excluded, such as gene variants receiving a lower weight or coefficient than other gene variants. A transformation may be learned between the basis factors of the meta-patients for each dataset and applied to each patient of the plurality of patients in the target dataset to generate the adapted dataset. The adapted dataset may be a matrix of patients by adapted gene variants or only the basis embeddings of the adapted gene variants for each patient.

Adaptation Pipeline for Imaging datasets

When the source and target datasets include imaging datasets the pipeline may be configured as follows:

Preprocessing may include padding images of each dataset to be to the same dimensions and/or normalizing the datasets such that any underlying image features have unit-variance.

Dimensionality transformation may include selecting a singular value decomposition (SVD), PCA transformation, or another dimensionality reduction algorithm, where the number of SVD basis is the minimum of the number of features representing image characteristics of that dataset and the number of patients in the dataset considered, and applying the SVD transformation to the datasets to identify SVD factors, principal component factors, adaptation factors, or encoding basis and SVD embeddings or encoded values. One example may include images of a human face, where an image may comprise eigenvector(s) for identifiable features of the eye(s), the nose, the mouth, and/or other facial features. Normalization may include ensuring that at least an eigenvector, even if null, exists for each facial feature. Another example may include a pathology slide image, where an image may include eigenvectors representative of cellular characteristics, such as the type of cell (tumor, stroma, epithelium, fat, lymphocyte, or other types), the state of the cell (regular shape, irregular shape, living, decaying, or other states), and/or other pathology imaging features. Other imaging techniques may be represented with a corresponding set of eigenvectors to capture any elements or characteristics of the image, including the types of tissues present, bone structure, blood vessels present, regions with contrast dyes, density of a bone, or other imaging characteristics, Imaging eigenvectors may represent how each image differs from the mean image of the dataset. In one example, the eigenvalues associated with each eigenvector represent how much the images in the dataset vary from the mean image in that direction.

Alignment Optimization may include identifying a non-convex optimization formulation, a corresponding objective function to minimize, a relaxation process for the objective function such that the objective function is convex. In one example, the non-convex optimization may minimize the mean square error between the transformed target factors and the source factors with a matrix-rank penalization on the transformation between the source and target factors, and the relaxation may entail minimization of the mean square error between the transformed target factors and the source factors with a trace-norm penalization on the transformation between the source and target factors, which can be solved using a proximal gradient descent method, where the posed method iteratively (i) minimizes the Euclidean distance between the transformed source factors and target factors by moving in the direction of steepest descent and (ii) applies the proximal operator for the trace-norm of the transformation to the output of the previous descent step, till convergence of the iterative method. In another example, the non-convex optimization may minimize the mean square error between the transformed target factors and the source factors with a hard constraint on the matrix-rank of the transformation between the source and target factors, which can be solved using a projected gradient descent method, where the posed method iteratively (i) minimizes the Euclidean distance between the transformed source factors and target factors by moving in the direction of steepest descent and (ii) applies the projection operator for the low matrix-rank approximation of the transformation to the output of the previous descent step, till convergence of the iterative method. The output of the proximal gradient descent method or the projected gradient descent method.

Reverse transformation may include selecting an inverse SVD transformation or decoder and applying the inverse SVD transformation or decoder to the corrected target dataset to generate the adapted dataset. In other examples, decoding or reverse transformations may be performed using inverse NMF, PCA, or other matrix factorizations.

In one example, imaging features may include features as disclosed in U.S. Pat. No. 10,957,041, issued Mar. 23, 2021, and titled “Determining Biomarkers from Histopathology Slide Images,” which is incorporated by reference herein for all purposes.

Adaptation Pipeline for Clinical Information Datasets

When the source and target datasets include clinical information datasets the pipeline may be configured as follows:

Preprocessing may include identifying the word or feature sets for each of the records of the dataset capture a feature or word vector, and then padding the words or features with null entries across each database to the same dimensions and/or normalizing the datasets such that any underlying clinical information features have unit-variance.

Dimensionality transformation may include selecting a singular value decomposition (SVD), PCA transformation, or another dimensionality reduction algorithm, where the number of SVD basis is the minimum of the number of features representing image characteristics of that dataset and the number of patients in the dataset considered, and applying the SVD transformation to the datasets to identify SVD factors, principal component factors, adaptation factors, or encoding basis and SVD embeddings or encoded values. One example may include features of a clinical information patient database, where clinical information may comprise eigenvector(s) for medical concepts as they appear in the electronic medical record. Normalization may include ensuring that at least an eigenvector, even if null, exists for each medical concept. Another example may include the number of times a word appears in records for each patient of a clinical information patient database, where words may include terms or phrases associated with symptoms, testing results, diagnosis, treatments, therapies, outcomes and/or other clinical information of a patient.

Alignment Optimization may include identifying a non-convex optimization formulation, a corresponding objective function to minimize, a relaxation process for the objective function such that the objective function is convex. In one example, the non-convex optimization may minimize the mean square error between the transformed target factors and the source factors with a matrix-rank penalization on the transformation between the source and target factors, and the relaxation may entail minimization of the mean square error between the transformed target factors and the source factors with a trace-norm penalization on the transformation between the source and target factors, which can be solved using a proximal gradient descent method, where the posed method iteratively (i) minimizes the Euclidean distance between the transformed source factors and target factors by moving in the direction of steepest descent and (ii) applies the proximal operator for the trace-norm of the transformation to the output of the previous descent step, till convergence of the iterative method. In another example, the non-convex optimization may minimize the mean square error between the transformed target factors and the source factors with a hard constraint on the matrix-rank of the transformation between the source and target factors, which can be solved using a projected gradient descent method, where the posed method iteratively (i) minimizes the Euclidean distance between the transformed source factors and target factors by moving in the direction of steepest descent and (ii) applies the projection operator for the low matrix-rank approximation of the transformation to the output of the previous descent step, till convergence of the iterative method. The output of the proximal gradient descent method or the projected gradient descent method.

Reverse transformation may include selecting an inverse SVD transformation or decoder and applying the inverse SVD transformation or decoder to the corrected target dataset to generate the adapted dataset. In other examples, decoding or reverse transformations may be performed using inverse NMF, PCA, or other matrix factorizations.

In one example, clinical information features may include features as disclosed in US Patent Publication No. 2021/0090694, published Mar. 25, 2021, and titled “Data Based Cancer Research and Treatment Systems and Methods,” which is incorporated by reference herein for all purposes.

Detailed Spin Adaptation Pipeline Stages

In one example, the spin adaptation pipeline may be incorporated into a genomic sequencing laboratory. An exemplary genomic sequencing laboratory may include a number of patient cohorts having next generation sequencing results for RNA, DNA, and imaging results from pathology review, X-Rays, MRIs, or CT scans. Cohorts may be based on a diagnosis for the cancer patient, treatments the patient has undergone, is currently undergoing, or potential treatments the patient may qualify for. Additional cohorts may be generated. Exemplary cohort generation techniques are described in US Patent Publication No. 2021/0090694, published Mar. 25, 2021, and titled “Data Based Cancer Research and Treatment Systems and Methods,” incorporated by reference herein for all purposes.

The spin adaptation pipeline may include an algorithm for generating variance stabilized count data from two RNA-seq count datasets, namely source and target datasets, may include an algorithm for learning the low matrix-rank correction transformation between the source factors and the target factors, may apply the correction transformation to the latent space embeddings or encodings of the target dataset, and then reverse transform or decode the corrected target embeddings to return the adapted target dataset. The algorithms provided below are exemplary and provided to illustrate an effective approach for each step; however, it should be understood to one of ordinary skill in the art that additional algorithms may similarly be utilized without departing from the scope of the disclosure, herein. Specifically, as outlined in Algorithm 1 below, a PCA space may be selected as the latent space, and transformations may be learned between PCA factors of the source dataset and the target dataset. However, for another pair of datasets, sparse PCA basis may provide more representative basis factors, or SVD, NMF, or another dimensionality reduction/basis learning algorithm.

A spin adaptation pipeline, the spin adaptation method or Algorithm 1, may be understood alongside a glossary of the relevant data structures and parameters.

In one example, where an entity has access to both the source and target datasets, the Algorithm 1 steps 1-8 (outlined below) may be incorporated into each stage of an exemplary pipeline as described herein. For example: Step 1 of spin adaptation may be performed at preprocessing stage of an exemplary pipeline; Steps 2-3 of spin adaptation may be performed at dimensionality transformation stage of an exemplary pipeline; Steps 4-6 of spin adaptation may be performed at the alignment optimization stage of an exemplary pipeline; Step 7 may be performed at the reverse transformation stage of an exemplary pipeline; and Step 8 may represent the output from an exemplary pipeline. In another example, the steps outlined above may be shifted between adjacent stages of an exemplary pipeline to balance or promote pipeline throughput based on calculation costs at each stage.

In another example, where an entity has access to only the source dataset and another entity has access to the target dataset, the Algorithm 1 steps 1-8 (outlined below) may be incorporated into each stage of two exemplary pipelines as described herein. For example: Step 1 of spin adaptation may be performed at preprocessing stage of both of the two exemplary pipelines, once for each entity; Steps 2-3 of spin adaptation may be performed at dimensionality transformation stage of each exemplary pipeline; Steps 4-6 of spin adaptation may be performed at the alignment optimization stage of an exemplary pipeline, wherein one entity received the results from Steps 1-3 of the another entity; Step 7 may be performed at the reverse transformation stage of an exemplary pipeline; and Step 8 may represent the output from an exemplary pipeline. In another example, the steps outlined above may be shifted between adjacent stages of an exemplary pipeline to balance or promote pipeline throughput based on calculation costs at each stage.

Glossary

The data structures employed in the following algorithms are defined and outlined below with the dimensionality of each structure may be stated in terms of p, the number of genes; nₛ, number of samples in source dataset; nₜ, number of samples in target dataset; d₁ or dₛ, dimensionality of source latent space; or d₂ or dₜ, dimensionality of target latent space.
a. Xₛ∈R^{p×n}^{ₛ}: The input or train source dataset.
b. Xₜ∈R^{p×n}^{ₜ}: The input or train target dataset.
c. Xₛₕ∈R^{p×n}^{ₜ}: The held-out source dataset.
d. X_{s,i}∈R^{p}: The i-th column of Xₛ.
e. X_{t,i}∈R^{p}: The i-th column of Xₜ.
f. mₛ∈R^{p}: The empirical mean or empirical gene-wise mean of source dataset.
g. mₜ∈R^{p}: The empirical mean or empirical gene-wise mean of target dataset.
h. sₛ∈R^{p} The empirical gene-wise variance of source dataset.
i. sₜ∈R^{p} The empirical gene-wise variance of target dataset.
j. Cₛ∈R^{p×p}: The empirical covariance of source dataset.
k. Cₜ∈R^{p×p}: The empirical covariance of target dataset.
l. Uₛ∈R^{p×d}^{₁}: Principal Component factors for source dataset.
m. Uₜ∈R^{p×d}^{₂}: Principal Component factors for target dataset.
n. A∈R^{d}^{₁}^{×d}^{₂}: Transformation matrix.
o. X_{sc}∈R^{p×n}^{ₛ}: The corrected output source dataset.
p. X_{tc}∈R^{p×n}^{ₜ}: The corrected output target dataset.
q. X(i,j) The i-th row and j-th column of any matrix X.
r. v(i) The i-th entry of any vector v.
s. Fₜ Classifier trained on the target dataset.

The parameters α and λ correspond to step size and regularization parameters for the iterative algorithm Fit (Algorithm 3). The parameter varianceₙₒᵣₘ is a boolean variable, which determines if the adaptation step (Step 4, Algorithm 1) entails variance-normalization of the source dataset (see Algorithm 4 for details).

Algorithm 1 spin adaptation

function Adapt (Xₛ, Xₜ, d₁, d₂, α, λ)
Compute empirical mean vectors

| | |
|---|---|
| 1. | ${m}_{s} ← \frac{1}{{n}_{s}} {Σ}_{i = 1}^{{n}_{s}} {X}_{s , i} , {m}_{t} ← \frac{1}{{n}_{t}} {Σ}_{i = 1}^{{n}_{t}} {X}_{t , i}$ |

Compute empirical covariance matrices, such as shifting the mean
264 of FIG. 2B.

| | |
|---|---|
| 1. | ${C}_{s} ← \frac{1}{{n}_{s} - 1} ( {X}_{s} - {m}_{s} ) {( {X}_{s} - {m}_{s} )}^{T}$ |
| 2. | ${C}_{t} ← \frac{1}{{n}_{t} - 1} ( {X}_{t} - {m}_{t} ) {( {X}_{t} - {m}_{t} )}^{T}$ |

Compute PCA factors

| | |
|---|---|
| 1. | Uₛ ← Eigenvectors(Cₛ), Uₛ ← Uₛ[., 1:d₁] |
| 2. | Uₜ ← Eigenvectors(Cₜ), Uₜ ← Uₜ[., 1:d₂] |

Learn transformation between PCA factors

| | |
|---|---|
| 1. | A ← LearnAdapt(Uₛ, Uₜ, α, λ) |

Compute PCA embeddings

| | |
|---|---|
| 1. | \tilde{X}ₜ ← Uₜ^{T}(Xₜ − mₜ) |

Apply transformation to target PCA embeddings, such as
rotating the basis axis 262 of FIG. 2B.

| | |
|---|---|
| 1. | \tilde{X}_{tc} ← A \tilde{X}ₜ |

Apply inverse PCA transform to corrected target embeddings

| | |
|---|---|
| 1. | X_{tc} ← Uₜ \tilde{X}ₜ + mₜ |

Return Xₛ, X_{tc}

The parameters α and λ correspond to step size and regularization parameter for the iterative algorithm LearnAdapt (Algorithm 2), which is detailed below.

The spin adaptation pipeline may perform optimally when the source and target datasets comprise matching population compositions across datasets (same ratio of tissue subtypes in source and target datasets). The data model effectively translates differences across datasets due to technical biases, and thus, Algorithm 1 is designed to capture, or learn, the heterogeneity between input datasets and correct for it. If the population composition varies across source and target datasets, Algorithm 1 may capture biological variability across datasets as a technical bias, which may add noise to the estimate of A in Step 4, Algorithm 1, which is implemented in Algorithm 3, as explained later. In one embodiment, a biological variability correction bias may be calculated and applied at this step to counter any biological variability, such as by having one laboratory sequence samples from each biological source and calculate, using the same methods herein, based upon the perceived technical bias. In this manner, the laboratory does not exhibit technical bias because the procedures and methodologies for sequencing are shared between the datasets and the bias captured and corrected includes only the biological variance. The pipeline may also perform optimally when the raw count data has undergone a variance stabilization transformation, before being passed as input to Algorithm 1. The cross-dataset corrections in Algorithm 1 are based on learning a transformation between the PCA factors of source and target datasets. If the input datasets are not variance stabilized, the high-expression genes would dominate the computation of PCA factors for each dataset because of the mean-dependence of variance for negative binomial distribution. As a result, the transformation A would be biased and capture the technical biases among high-expression genes. Furthermore, variance stabilization ensures that the features in each input dataset have variance within similar scale, which is important since we learn the corrections on the PCA factors and not the PCA embeddings or RNA-seq samples, and the computation of the PCA factors is sensitive to mismatch in the variance of the various features.

Learning Transformation Between PCA Factors

In Step 4, Algorithm 1, a transformation between PCA factors of the source dataset (source factors) and PCA factors of the target dataset (target factors) is learned. To learn the transformation, a non-convex optimization problem is considered and then a convex relaxation of the non-convex problem is identified. This convex relaxation enables an effective computational approach to learn a transformation between the source factors and the target factors.

Objective function for learning a transformation. The objective function is based on Euclidean distance between the transformed source factors and the target factors, as follows

$$1. {A}_{r}^{*} = {‖ {U}_{t} A - {U}_{s} ‖}_{F} + λ rank ( A ) , \qquad ( 1 )$$

where Uₜ and Uₛ represent the target factors and the source factors, respectively, A represents the transformation matrix, λ∈R⁺ represents regularization parameter, and rank(A) represents the matrix-rank of A. In the first term of equation (1), it can be seen that the i-th column of the transformation matrix A determines what linear combination of the columns of Uₜ best approximates the i-th column of Uₛ, where i=1, 2, . . . , d₁. In other words, the technical biases between target dataset and source dataset are modeled using a linear combination of target factors, and then approximating each source factor with the linear combination of target factors. In this way, technical biases in a dataset can be represented by a linear combination of PCA factors and the approximation of the linear combinations of PCA factors may be used to transform the target factors to match the source.

The second term of equation (1), the matrix-rank penalization term, may favor low matrix-rank transformations A for matching the transformed target factors with their source counterparts (source factors). The penalization term, in one example, may be similar to a regularization term in a LASSO objective function, and operate to better identify sparse solutions. The degree of matrix-rank penalization in equation (1) depends on λ. As λ increases, the preference for low matrix-rank transformation increases, and vice versa. When λ=0, the problem in equation (1) reduces to learning any transformation that matches the corrected target factors with the source factors.

The second term in equation (1) penalizes matrix-rank, but it is non-convex, which makes equation (1) hard to evaluate. By substituting rank(A) with the trace norm of A, ∥A∥, which is the convex envelope of rank(A), the following convex relaxation for the non-convex optimization problem in (1) may be generated,

$$1. {A}^{*} = {‖ {U}_{t} A - {U}_{s} ‖}_{F} + λ {‖ A ‖}_{*} . \qquad ( 2 )$$

This convex relaxation avoids the difficulties of deriving a solution to a non-convex problem and generates an efficient routine for computing A*.

Optimization. By setting g(A)=∥UₜA−Uₛ∥_{F} and h(A)=∥A∥*, the objective function in equation (2) may be expressed as,

$$1. {A}^{*} = g ( A ) + λ h ( A ) . \qquad ( 3 )$$

Further setting λ=0 reduces the objective function to the first term, which is convex and differentiable, and thus it can be minimized on R^{d×d} using the gradient descent method. Given the gradient ∇g(A), an initial estimate A⁽⁰⁾, and step size α, the objective function in equation (3) may be generated for λ=0 using the iterative application of

$$1. {A}^{( k + 1 )} = {A}^{( k )} - α ∇ g ( A ) , \qquad ( 4 )$$

for k=0, 1, 2, 3, . . . until convergence. Further optimization may be realized by setting \tilde{g}(A^{(k+1)}) to be a quadratic approximation of g(A^{(k+1)}) from point A^{(k)}, with the hessian replaced by an identity map and then scaled by

$$\frac{1}{α} ,$$

defined as

$$\tilde{g} ( {A}^{( k + 1 )} ) \approx g ( {A}^{( k )} ) + 〈 ∇ g ( {A}^{( k )} ) , {A}^{( k + 1 )} - {A}^{( k )} 〉 + \frac{1}{2 α} {‖ {A}^{( k + 1 )} - {A}^{( k )} ‖}_{F}^{2} . \qquad ( 5 )$$

With matrix A^{(k)}, another matrix A^{(k+1)} may be generated such that g(A^{(k+1)}) is minimized. Solving ∇\tilde{g}(A^{(k+1)})=0 may identify an optimal A^{(k+1)} as A^{(k+1)}=A^{(k)}−α∇g(A), which is the gradient descent step in equation (4). Thus, the gradient descent step in equation (4) follows from the minimization of a quadratic approximation of function g at point A^{(k)}.

Gradient descent can be used to evaluate equation (3) for λ=0 because g(A) is convex and differentiable. For λ≠0, equation (3) may not be evaluated using gradient descent, because h(A) is non-differentiable. This may be cured by computing the proximity operator of h and minimizing g(A)+λh(A) using a proximal algorithm.

Proximal operators and trace norm. The proximal operator of a function f: R^{n×n}→R with parameter θ is defined by

$$1. p r o {x}_{f , θ} ( Y ) = f ( Z ) + \frac{1}{2 θ} {‖ Z - Y ‖}_{F}^{2} . \qquad ( 6 )$$

Evaluation of the proximal operator with parameter θ for the trace-norm function is represented as prox_{h,θ}(y),

$$1. p r o {x}_{h , θ} ( Y ) = h ( Z ) + \frac{1}{2 θ} {‖ Z - Y ‖}_{F}^{2} \\ i . = {‖ Z ‖}_{*} + \frac{1}{2 θ} {‖ Z - Y ‖}_{F}^{2} . \qquad ( 7 )$$

A closed-form solution of equation (7) may be computed by evaluating an SVD transform Y=UΣV^{T}, followed by soft thresholding of the element-wise entries of Σ, where columns of U∈R^{n×n} contain the eigenvectors of YY^{T}, columns of V∈R^{n×n} contain the eigenvectors of Y^{T}Y, and entries of diagonal matrix Σ are square roots of the eigenvalues of YY^{T}. Specifically, the solution of equation (7) is the output of Algorithm 2, outlined below.

Algorithm 2 Proximal operator for the trace-norm function

function ProximalOperator (Y,θ)
a. U, Σ, V^{T} ← SVD( Y)
b. for i ← 1,2, . . . , n
c. Σ_{i,i} ← {0, |Σ_{i,i}| ≤ θ; Σ_{i,i} − θ sign(Σ_{i,i}), |Σ_{i,i}| > θ;
d. Ŷ ← U Σ V^{T}
e. return Ŷ

Therefore, the algorithm, LearnAdapt (Step 5, Algorithm 1), the transformation matrix between the source factors and the target factors is learned in a convex solution space. The computational procedures outlined minimize the objective in equation (2), by iterative application of (a) the gradient descent step in equation (4), and (b) the trace-norm proximal operator in equation (7), which is evaluated by Algorithm 2, until convergence. Algorithm 3 outlines one exemplary embodiment with each of these steps included.

Algorithm 3 Transformation between source factors and target factors

```
  function LearnAdapt (Uₛ, Uₜ, α, λ)
a. k=0
b. Initialize A⁽⁰⁾
c. repeat
  {Gradient descent}
  Â^{(k)} ← A^{(k)} − α∇g(A^{(k)})
  {Proximal operator}
i.A^{(k + 1)} ← ProximalOperator ( Â^{(k)}, αλ)
  k ← k + 1
a. until convergence
b. return A^{(k)}
```

Algorithm 4: SpinAdapt algorithm

```
    function Main(Xₜ, Xₛ, Xₛₕ(optional), Fₜ , α, λ, varianceₙₒᵣₘ)
1. Compute data factors for source:
  Uₛ, mₛ, sₛ = Factors(Xₛ).
2. Compute data factors for target:
  Uₜ, mₜ, sₜ = Factors(Xₜ).
3. Fit (train) SpinAdapt correction model:
  A ← SpinAdapt.Fit(Uₛ, Uₜ, α, λ).
4. Transform source dataset:
      X_{sc} ← SpinAdapt.Transform
      (Xₛ, Xₛₕ(optional), A, Uₛ, mₛ, sₛ, Uₜ, m, sₜ, varianceₙₒᵣₘ).
5. Apply target-trained predictor Fₜ :
  y_{sc} ← Fₜ(X_{sc}).
Return predictions y_{sc}
```

Algorithm 5: Compute data factors, gene-wise means and variances

function Factors(Xₑ):

| | |
|---|---|
| 1. | Define nₑ: = number of columns in Xₑ, p: = number of rows |
| | in Xₑ, dₑ: = min(nₑ, p). |
| 2. | Compute empirical mean vector. |

${m}_{e} ← \frac{1}{{n}_{e}} {Σ}_{i = 1}^{{n}_{e}} {X}_{e , i}$

| | |
|---|---|
| 3. | Compute empirical variance vector. |

${s}_{e} ← \frac{1}{{n}_{e} - 1} {{Σ}_{i = 1}^{{n}_{e}} ( {X}_{e , i} - {m}_{e} )}^{2}$

| | |
|---|---|
| 4. | Compute empirical covariance matrix. |

${C}_{e} ← \frac{1}{{n}_{e} - 1} ( {X}_{e} - {m}_{e} ) {( {X}_{e} - {m}_{e} )}^{T}$

| | |
|---|---|
| 5. | Compute eigenvectors of the empirical covariance matrix. |

Uₑ ← Eigenvectors(Cₑ)

| | |
|---|---|
| 6. | Retain top dₑ eigenvectors. |

Uₑ ← Uₑ[1:dₑ]

| | |
|---|---|
| 7. | Return Uₑ, mₑ, sₑ |

Algorithm 6: Learn transformation from source to target factors (Fit)

```
  This algorithm may be used for executing Step 3, Algorithm 4, which is
essentially a solution to the optimization problem in (2). We propose the
use of the projected gradient descent algorithm to evaluate (2), which is
an iterative application of the following descent step:
A^{(k+1)} = P_{λ}( A^{(k)} − α∇g(A) ),
for k = 0, 1, 2, 3, . . . till convergence.
function SpinAdapt.Fit(Uₛ, Uₜ, α, λ)
k=0
Initialize A⁽⁰⁾
repeat
  a. {Gradient descent}
      Â^{(k)} ← A^{(k)} − α∇g(A^{(k)})
  b. {Projection Step}
    A^{(k+1)} ← P_{λ}(Â^{(k)})
  c. k ← k + 1
until convergence
return A^{(k)}
```

Algorithm 7: Pseudocode for SpinAdapt.Transform (Adapt the source dataset)

```
  This algorithm may be used to execute Step 4, Algorithm 4, where the batch-
biased dataset is corrected using the transformation A. Details are outlined in Algorithm
7, as follows. In Step 1, Algorithm 7, the held-out source dataset Xₛₕ is selected for
correction, if provided. If held-out evaluation data (Xₛₕ) is not provided, the train source
dataset Xₛ is selected for correction. In Step 2, if the input parameter varianceₙₒᵣₘis set
to True, the variance of each gene in the source dataset is matched to variance of the
corresponding gene in the target dataset. In Step 3, the PCA embeddings of each
source sample are computed. In Step 4, the computed PCA embeddings are corrected,
using the transformation matrix A. In Step 5, the corrected PCA embeddings are
transformed to the gene expression space. Finally, the corrected source gene
expression profiles are returned in Step 6.
  Function
SpinAdapt.Transform (Xₛ, Xₛₕ(optional), A, Uₛ, mₛ, sₛ, Uₜ, mₜ, sₜ, varianceₙₒᵣₘ)
    1. Select the dataset for transformation:
      If Xₛₕ is Null:
        Xₒ:= Xₛ.
      Else:
        Xₒ:= Xₛₕ.
  2. Gene-wise variance normalization:
      If varianceₙₒᵣₘ is True:
        For i = 1,2, . . . , p
          For j = 1,2, . . . , nₛ
            Xₒ(i,j) ← ({square root over ((sₜ(i))} (Xₒ(i,j) − mₛ(i) ) / \sqrt{sₛ(i)}) + mₛ(i).
  3. Compute source PCA embeddings:
      \tilde{X}ₛ ← Uₛ^{T} (Xₒ − mₛ).
  4. Correct source PCA embeddings:
      \tilde{X}_{sc} ←A^{T} \tilde{X}ₛ.
  5. Map corrected source PCA embeddings to the gene expression space:
      X_{sc} ← Uₜ \tilde{X}_{sc} + mₜ.
  6. Return X_{sc}.
```

SpinAdapt inputs source and target expression datasets for training, corrects the batch-biased source expression data, even when the source data is held-out from training, followed by evaluation of the target-trained predictor on the corrected source data. The algorithm, as outlined in Algorithm 4, can be broken down into several main steps: computation of source and target data factors from source and target datasets in Steps 1 and 2, respectively, estimation of a low-rank affine map between source and target PCA basis in Step 3, adaptation (correction) of the source dataset in Step 4, and finally, evaluation of the target-trained predictor on adapted source dataset in Step 5. Notably, Step 4, Algorithm 4 can adapt source dataset Xₛₕ that is held-out from the training source dataset X. Steps 1 and 2 are executed using Algorithm 5, whereas Steps 3 and 4 are executed using Algorithms 6 and 7, respectively. These steps are explained next in further detail.

In Step 1, Algorithm 4, data factors are computed for the source dataset Xₛ, where the data factors comprise of the PCA basis Uₛ, gene-wise means mₛ, and gene-wise variances sₛ of the source dataset. The details for the computation of these data factors are outlined in Algorithm 5, where the gene-wise means and variances are computed in Steps 2-3, whereas the PCA basis are computed in Steps 4-6. Similarly, data factors are computed for the target dataset in Step 2, Algorithm 4, where the data factors entail the PCA basis Uₜ, gene-wise means mₜ, and gene-wise variances sₜ for the target dataset Xₜ. The gene-wise means and variances mₛ, mₜ, sₛ, sₜ are used in the correction step (Step 4, Algorithm 4), whereas the PCA basis Uₛ and Uₜ are used in the train and correction steps (Steps 3-4, Algorithm 4). The usage of statistics sₛ and sₜ in Step 4, Algorithm 4 is optional depending on the boolean value of varianceₙₒᵣₘ, as we explain later.

Notably, Algorithm 4 does not require simultaneous access to sample-level patient data in source and target datasets at any step. Computation of source data factors in Step 1 needs access to Xₛ only, whereas computation of target data factors in Step 2 needs access to Xₜ only. Training in Step 3 only requires access to the PCA basis of source and target datasets. Since the PCA basis cannot be used for recovery of sample-level patient data, the basis are privacy-preserving. Adaptation in Step 4 requires access to the source expression data Xₛ, linear map A, and the data factors of both datasets, without requiring access to the target dataset Xₜ.

Algorithm Details: Learning transformation between PCA factors

In Step 3, Algorithm 4, we learn a low matrix-rank transformation between PCA factors of the source dataset and PCA factors of the target dataset. We pose a non-convex optimization problem to learn the transformation, and then we present an effective computational approach to solve it, as we explain next.

Objective function for Step 3, Algorithm 4. The objective function is based on Frobenius norm between transformed source PCA basis Uₛ A and the target PCA basis Uₜ, as follows

$${A}_{r}^{*} = {arg min}_{A} {‖ {U}_{s} A - {U}_{t} ‖}_{F} , s . t . rank ( A ) \leq λ , \qquad ( 1 )$$

where A represents the transformation matrix, λ represents the matrix-rank constraint, and rank(A) represents the matrix-rank of A. In the main term, it can be seen that the i-th column of the transformation matrix A determines what linear combination of the columns of Uₛ best approximates the i-th column of Uₜ, where i=1,2, . . . , dₜ. Therefore, the intuition behind the main term is to approximate each target factor using some linear combination of source factors.

The inequality constraint in equation (1) is a matrix-rank penalization term, which restricts the solution space of A to matrices with matrix-rank less than A. The rank constraint is reminiscent of sparse constraint in sparse recovery problems, where the constraint restricts the maximum number of non-zero entries in the estimated solution, thereby reducing the sample complexity of the learning task. Similarly, in equation (1), the constraint restricts the maximum matrix-rank of A, making the algorithm less prone to overfitting, while decreasing the sample requirement of learning the affine map from source to target factors. However, the problem posed in equation (1) turns out to be non-convex, and thus hard to solve. We employ traditional optimization techniques and derive an efficient routine for computing Aᵣ*, as follows in the next subsection.

Optimization solution for Step 3, Algorithm 4. Let g(A)=∥UₛA−Uₜ∥_{F}, and let S_{λ}={A: A∈R^{d}^{ₛ}^{×d}^{ₜ}, rank(A)≤A}. Then, the objective function in equation (1) can be re-written as

$${A}_{r}^{*} = {arg min}_{A} g ( A ) , s . t . A ∈ {S}_{λ} . \qquad ( 2 )$$

Gradient descent can be used to minimize g(A) w.r.t. A because the function is convex and differentiable. In contrast, equation (2) cannot be evaluated using gradient descent, since the set of low-rank matrices S_{λ} is non-convex. However, we note that the Euclidean projection onto the set S_{λ} can be efficiently computed, which hints that equation (2) can be minimized using projected gradient descent, as we explain next. Let the Euclidean projection of a matrix A onto set S_{λ} be denoted by P_{λ}(A). Then, mathematically we have P_{λ}(A)=argmin_{z} {∥A−Z∥_{F}: Z∈S₂}. From the Eckart-Young Theorem, we know that P_{λ}(A) can be efficiently evaluated by computing the top λ singular values and singular vectors of A. The closed-form solution of P_{λ}(A) is given by the SVD transform U_{λ}Σ_{λ}V_{λ}^{T}, where columns of U_{λ} contain the top λ eigenvectors of AA^{T}, columns of V_{λ} contain the top λ eigenvectors of A^{T}A, and entries of the diagonal matrix Σ_{λ} are square roots of the top λ eigenvalues of AA^{T}. We are finally ready to present an algorithm for solving (2).

An exemplary spin adaptation pipeline configured for Algorithms 1-7 as described above may operate to first identify nature or biases, domain shifts, covariate shifts, or other dataset specific phenomenon, and then correct them between a source dataset and one or more target datasets. Exemplary embodiments as described may operate on datasets having differing numbers of samples, differing numbers of basis such as derived from genes or transcripts sequenced, and differing ratios of samples to basis.

### EXAMPLE 1

### Adaptation Between TCGA and SCAN-B Breast Cancer Datasets

A spin adaptation pipeline may homogenize Breast cancer RNA-Seq samples from TCGA (The Cancer Genome Atlas) and SCAN-B (Swedish Breast Cancer Cohort). In one example, both datasets may include approximately 800 untreated samples of primary breast cancer that were RNA-sequenced and include a matching PAM50 diagnostic IHC staining result for each sample. For homogenization performance comparison, a comparison of clustering performance of a spin adaptation engine with a homogenization approach that performs gene-wise z-score normalization of the two datasets may be performed, where the clusters are assigned to the PAM50 breast cancer subtypes (Luminal A, Luminal B, HER2+, Basal).

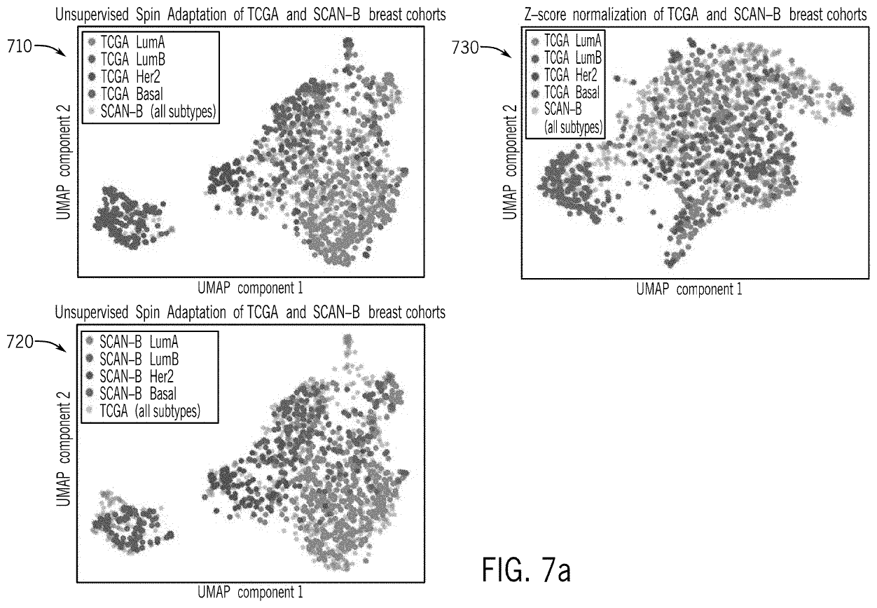

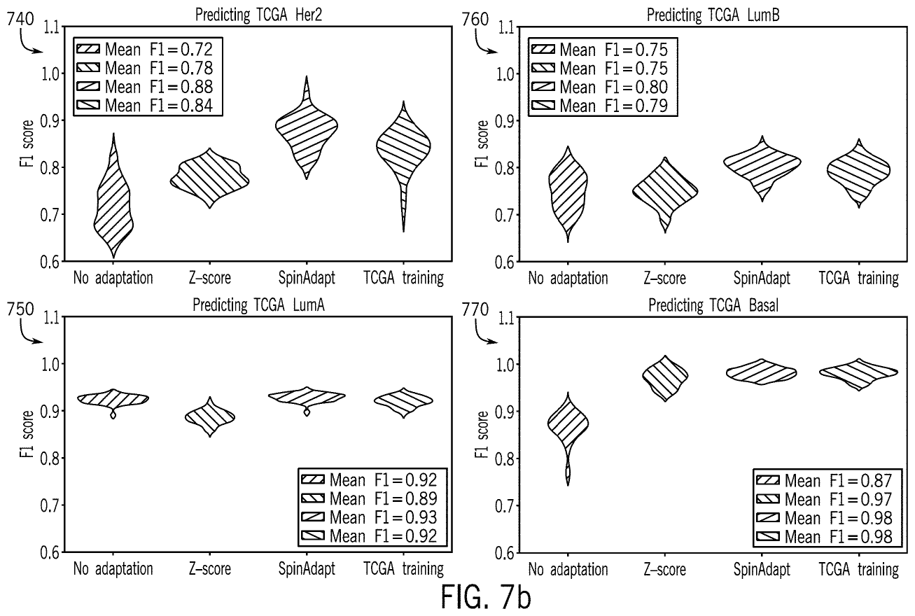

FIG. 7A illustrates spin adaptation normalization of SCAN-B and TCGA.

As depicted in plots 710 and 720, all four tissue subtypes (Luminal A, Luminal B, HER2+, Basal) cluster together across TCGA and SCAN-B.

As depicted in plot 730, which depicts Z-score normalization of SCAN-B and TCGA, Basal and Luminal B nicely cluster together across TCGA and SCAN-B. Luminal A and HER2 are not consistent.

Performance of the spin adaptation engine for the transfer of predictors across datasets included transferring PAM50 breast cancer subtype (Luminal A, Luminal B, HER2+, Basal) predictors trained on SCAN-B data to accurately predict subtypes from TCGA. First, the TCGA cohort (n=481 Luminal A, n=189 Luminal B, n=73 Her2+, n=168 Basal) is randomly split into two sets: TCGA-train and held-out TCGA-test (n=455 and n=456, respectively). Second, the TCGA-test set was homogenized with the SCAN-B cohort (n=837) using the spin adapt pipeline. Third, a molecular subtype predictor, based on a logistic regression model, was trained on homogenized SCAN-B RNA-Seq samples and used to predict the subtypes of TCGA-test RNA-Seg samples. Ridge regression penalty was employed with the logistic regression model and the regularization parameter was chosen in a five-fold cross-validation experiment on the SCAN-B dataset. For baseline comparison, similar predictors were also trained on competitor methods, including: (i) non-homogenized SCAN-B cohort, (ii) z-score normalized SCAN-B cohort, and (iii) TCGA-train cohort. Competitor methods were evaluated on the TCGA-test set. The experimental framework was iterated 25 times with randomly sampled training and testing cohorts from TCGA and SCAN-B. The explained procedure was repeated for each of the PAM50 breast cancer subtypes (Luminal A, Luminal B, HER2+, Basal), and the results are presented below.

FIG. 7B illustrates prediction results based on the spin adaptation normalization of FIG. 7A.

Plot 740 depicts HER2-f prediction results on TCGA-test, where an exemplary Spin Adaptation engine outperforms the competitor methods.

Plot 750 depicts Luminal A prediction results on TCGA-test, where an exemplary Spin Adaptation engine outperforms the competitor methods.

Plot 760 depicts Luminal B prediction results on TCGA-test, where an exemplary Spin Adaptation engine outperforms the competitor methods.

Plot 770 depicts Basal prediction results on TCGA-test, where an exemplary Spin Adaptation engine outperforms or ties the competitor methods.

### EXAMPLE 2

### Adaptation Between Breast Cancer Microarrays and RNA-seq Datasets

A spin adaptation pipeline may homogenize datasets having different sequencing methods, such as TCGA BRCA microarray and RNA-Seq datasets, consisting of paired samples from 583 patients, where the paired microarray and RNA-Seq datasets formed target and source datasets, respectively.

In one example, an entity which performs RNA microarray sequencing for patient samples may desire to collaborate with a second entity which performs NGS sequencing for patient samples and has developed an artificial intelligence engine which predicts a patient's outcome to treatments. However, the first entity may desire to maintain privacy of their patient dataset and not share their proprietary dataset with the second entity. As illustrated in FIG. 6, the second entity, or laboratory for NGS RNA-Seq, may pass an adaptation pipeline to the first entity, or RNA microarray sequencing, which may be incorporated into a pipeline framework or uploaded into a cloud-based platform. The second entity may pass adaptation factors generated from their dataset for inclusion into the adaptation pipeline of the first entity. The adaptation pipeline may be applied to the first entities proprietary dataset to generate adaptation factors and the adaptation factors of the two datasets may be applied to generate the correction transform and then apply the correction transform to generate an adapted dataset of the first entity which matches the data-specific nature of the second entity's dataset. The second entity may then pass the trained engine to the first entity which may be built into a machine learning framework or uploaded into a cloud-based platform. The first entity may then apply their adapted dataset to the trained engine to predict patient outcomes of their RNA microarray sequenced patients. Optionally, to provide insights into the accuracy of the model, such as validating performance, the first entity may pass their prediction results for their adapted dataset back to the second entity. Additional aspects of the trained machine learning model may be implemented as described below.

Wherein model performance may be bolstered by training on a combined dataset, the original dataset and the adapted datasets may uniformly and randomly divide the 583 subjects into 450 train and 133 test subjects to generate train and test samples that were paired across target and source datasets. Thus, the system may provide 450 train target and train source subjects for calculating the dataset specific phenomenon and 133 test target and test source subjects for validating the performance of the spin adaptation pipeline.

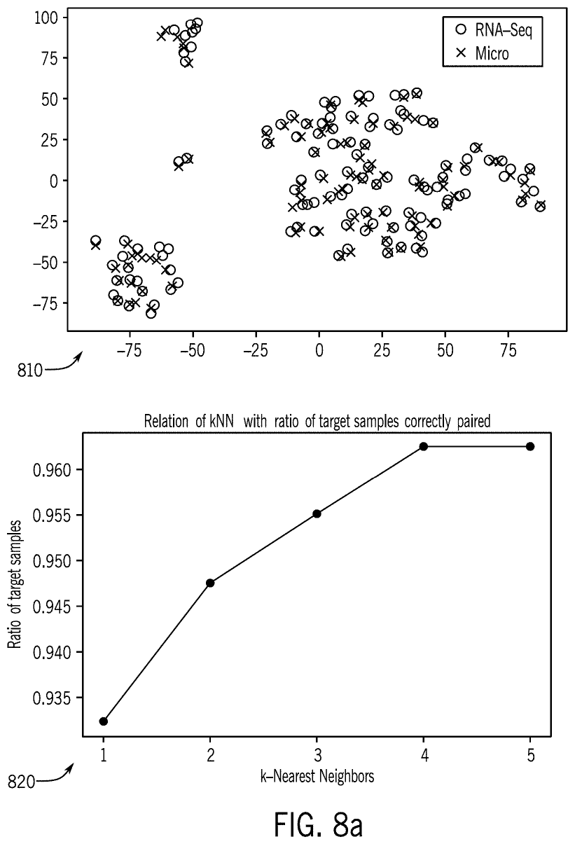

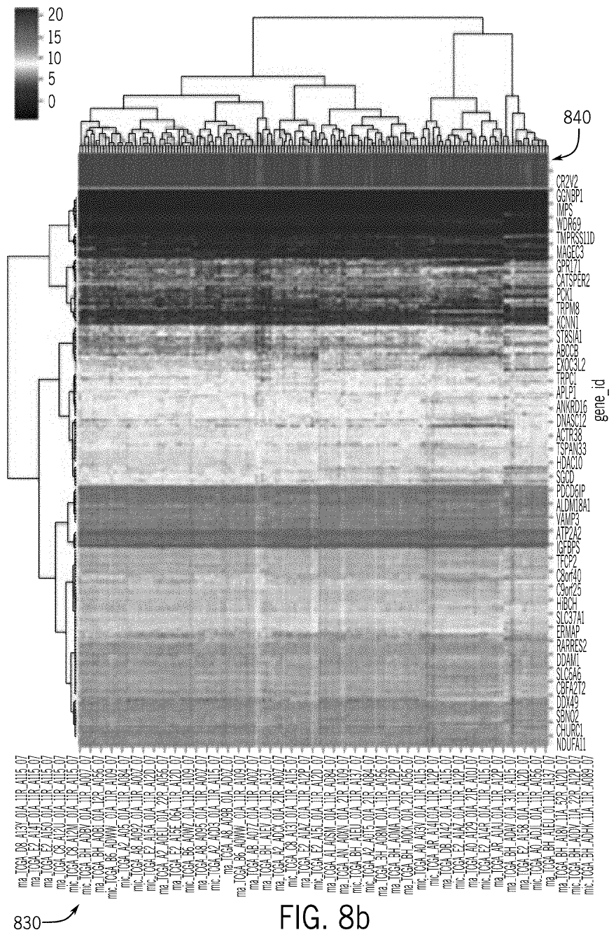

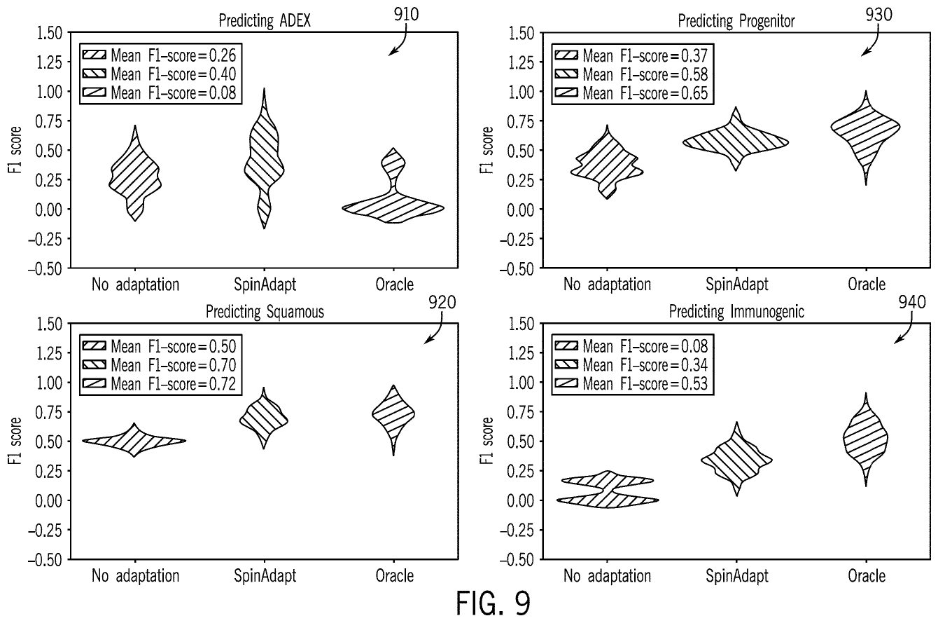

As a preprocessing step, variance of each gene in train source and train target datasets is computed, and correspondingly, the test target dataset is variance corrected such that genes in the target have matching variance to genes in the source. This variance matching preprocessing step is important when homogenizing datasets from different sequencing technologies. Next, we learned the unknown transformations (i.e., As, At, S*) from the train datasets and homogenized the test source and test target datasets using the spin adaptation algorithm. Following homogenization, the corrected test target and test source dataset were clustered using hierarchical clustering. The test source and test target samples were randomly dispersed among the various clusters, which empirically validates homogenization of the two datasets as illustrated in plot 810 of FIG. 8A. Furthermore, for the test target dataset, the k nearest neighbors of each test target sample in the test source dataset were calculated, for k=1,2,3,4,5. For each value of k, the ratio of source test samples with paired target samples were computed among its k nearest neighbors, and the relationship was computed and graphed in plot 820 of FIG. 8A. For k=1, more than 93% of the test source samples were correctly paired with their matching target samples. In other words, for more than 93% of the test RNA-seq target samples, the nearest neighbor was the paired Microarray source sample, further validating the homogenization algorithm.

The relationship of k with the ratio of test target samples correctly paired with test source samples. After applying the (test) arrays to the spin adaptation engine, 93% of the closest neighbors to a sequenced sample is its paired microarray.

FIG. 8B illustrates hierarchical clustering of corrected test source and target samples from FIG. 8A, according to an embodiment.

At plot 830, a dendrogram illustrates hierarchical clustering of samples between both datasets, where columns correspond to samples, rows correspond to genes, and the first row labeled as plot 840 corresponds to sample labels, such that the RNA-Seq and microarray samples are represented with green and purple colors, respectively. The charted samples 840 show that the RNA-Seq and microarray samples mix well together across various clusters in the dendrogram, empirically validating the homogenization performance of the spin adaptation algorithm.

### EXAMPLE 3

### Adaptation Between PACA-AU and PAAD-US Pancreatic Cancer Datasets

A spin adaptation pipeline may homogenize Pancreatic cancer RNA-Seq samples from PACA-AU and PAAD-US study cohorts having 69 and 121 untreated samples, respectively, of primary pancreatic cancer that were RNA-sequenced, The datasets define and include pancreatic cancer subtype labels: (1) squamous; (2) pancreatic progenitor; (3) immunogenic; and (4) aberrantly differentiated endocrine exocrine (ADEX) that correlate with histopathological characteristics from imagine slides of the sample's tumor.

The performance of the spin adaptation engine was analyzed for the transfer of predictors across datasets, including pancreatic cancer subtype (Squamous, Progenitor, Immunogenic, and ADEX) predictors trained on PAAD-US data to accurately predict subtypes from PACA-AU. The experimental procedure is explained as follows: First, the PACA-AU cohort (n=69) was randomly split into two sets: PACA-train and held-out PACA-test (n=34 and n=35, respectively). Second, the PACA-test set was homogenized with the PAAD-US cohort (n=121). Third, a molecular subtype predictor, based on a logistic regression model, was trained on PAAD-US samples and used to predict the subtypes of PACA-test RNA-Seq samples. An elastic net penalty was employed with the logistic regression model, and the regularization parameters were chosen in a three-fold cross-validation experiment on the PAAD-US (train) dataset. For baseline comparison, the logistic regression predictor was also evaluated on the non-homogenized PACA-test cohort. For further comparison, a similar logistic regression predictor was trained and evaluated on the PACA-train and the PACA-test datasets, which provided a performance analysis of a predictor trained and tested on the same PACA-AU cohort. Since such a predictor has access to train and test datasets from the same cohort, it is labeled as ‘Oracle’. The experimental framework was iterated 100 times, and the subtype prediction performance is quantified using an F-1 score for each subtype label (Squamous, Progenitor, Immunogenic, and ADEX) and each method (no adaptation, spin adaptation, Oracle), where F-1 score for each label is the harmonic mean of precision and recap for each subtype label.

FIG. 9 illustrates the results of an exemplary spin adaptation pipeline for Example 3.

Plot 910 depicts ADEX prediction results on PACA-test.

Plot 920 depicts Squamous prediction results on PACA-test.

Plot 930 depicts Progenitor prediction results on PACA-test.

Plot 940 depicts Immunogenic prediction results on PACA-test.

### EXAMPLE 4

### Adaptation Between Laboratory, TCGA, and Director's Challenge NSCLC (Non-Small Cell Lung Cancer) Datasets

A spin adaptation pipeline may homogenize NSCLC RNA-Seq samples from TCGA (n=495), RNA-expression micro-array samples from Director's Challenge (n=441), and RNA-Seq samples from Laboratory (n=212) study cohorts. These three datasets also include matching OS (Overall survival) for Director's Challenge dataset and matching PFS (Progression Free Survival) for TCGA and Laboratory datasets, as needed to train and transfer risk predictors from Laboratory cohort to the TCGA and Director's Challenge cohorts. A prediction was generated based upon a binary classification, where y=1 if the patient had a risk event at or before 24 months, y=0 if they did not have a risk event at or before 24 months.

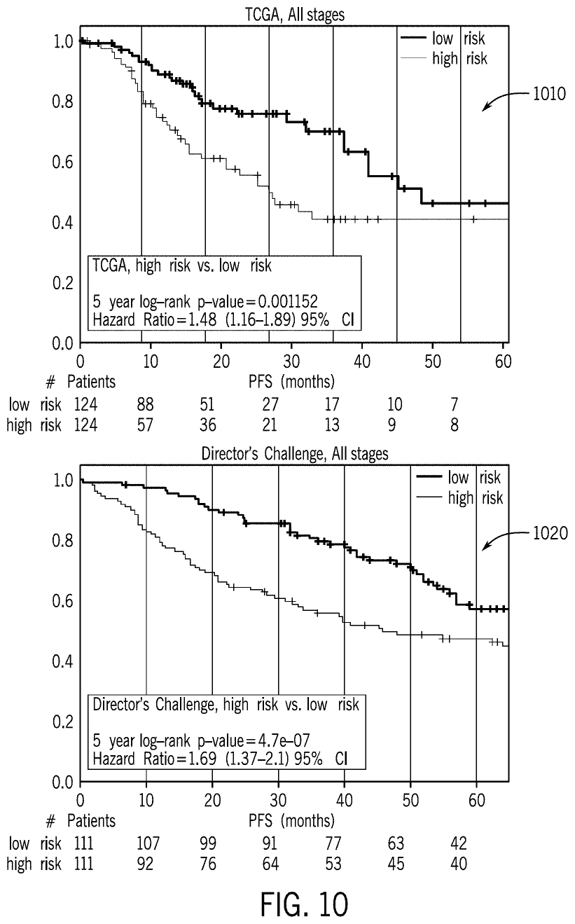

To analyze the performance of the spin adaptation engine for the transfer of risk predictors across datasets, predictors were trained on Laboratory data and then used to predict risk on the TCGA and the Director's Challenge cohorts. Specifically, a molecular risk predictor, based on a Random Forests model with 500 estimators and a maximum depth of 5, was trained on Laboratory RNA-Seq samples with a response label y, where y=1 if the patient had a risk event at or before 24 months, y=0 if they did not have a risk event at or before 24 months. Correspondingly, the test cohorts were homogenized with the train (Laboratory) cohort, and the trained predictor was evaluated on the homogenized test cohorts (TCGA and the Director's Challenge cohorts), where the test patients were predicted as either low-risk (y=0) or high-risk (y=1). Therefore, the transfer of the risk predictor enabled identification of the low-risk and the high-risk groups in the TCGA and the Director's Challenge cohorts, respectively. Survival analysis may be performed for each of the identified groups in the TCGA and Director's Challenge cohorts and plotted according to FIG. 10.

FIG. 10 illustrates survival curves for the low-risk and high-risk groups identified in the TCGA and Director's Challenge cohorts, from a model which was trained on a Laboratory cohort, where the TCGA and Director's Challenge cohorts are each provided to a spin adaptation pipeline with the Laboratory as source. Survival curves provide estimates on the duration of time until an event of interest occurs for a patient, which is cancer recurrence in this experimental setup.

For each of the test cohorts, the survival curves were plotted to illustrate low-risk and the high-risk patients in the TCGA and the Director's Challenge cohorts.

Plot 1010 depicts the survival curve for TCGA patients based on predictions of the Random Forest model trained and transferred from the Laboratory cohort.

Plot 1020 depicts the survival curve for Director's Challenge patients, based on predictions of the Random Forest model trained and transferred from the Laboratory cohort.

### EXAMPLE 5

### Algorithm Overview

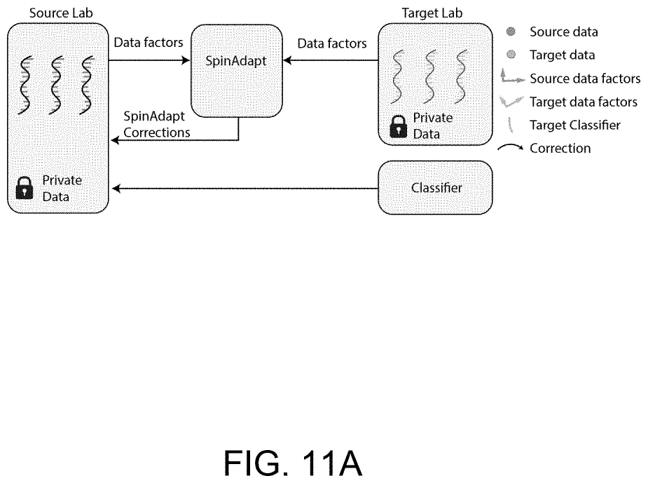

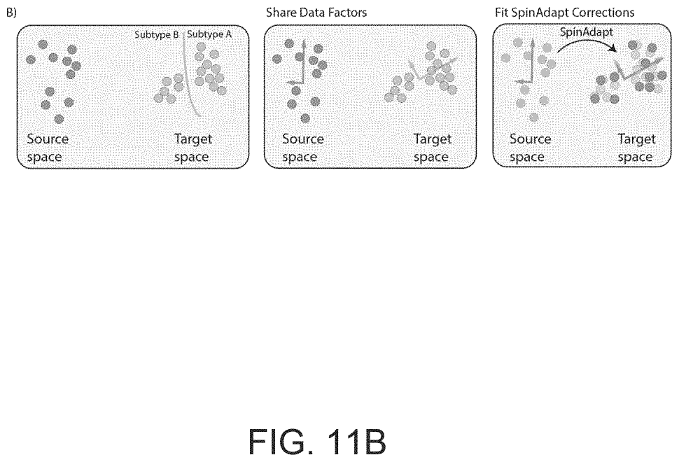

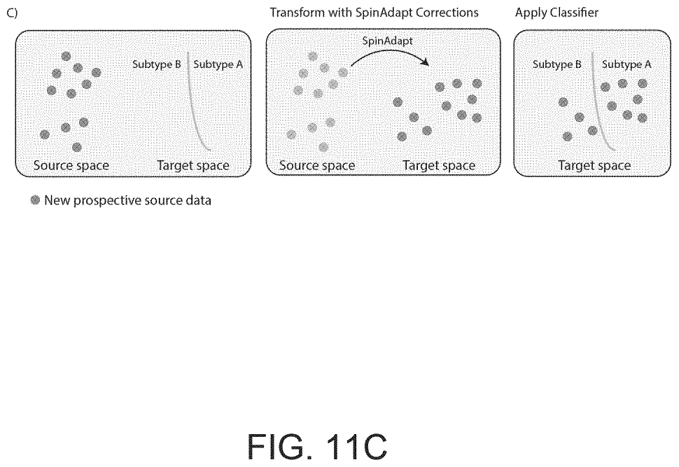

As represented in FIG. 11A, the present system and method represent a framework for transfer and validation of molecular predictors across platforms, laboratories, and varying technical conditions. That figure also illustrates that the present system and method also decouple the transfer of predictors from the transfer of training data, and while also enabling the transfer of predictors while preserving data privacy. Data factors, which are aggregate statistics of each dataset, neither convey Protected Health Information (PHI) nor allow reconstruction of sample-level data and thus can be shared externally. The present disclosure learns corrections between data factors of each dataset, followed by application of corrections on the biased expression dataset (source). This framework enables the correction of new prospective data, which has important implications as discussed later.

FIG. 11A depicts a privacy-preserving transfer of molecular models between a target lab and a source lab. For example, a target dataset with a trained classifier and protected assay data such as RNA data provides its privacy-preserving RNA factors and a molecular classifier to the present system and methods (“SpinAdapt”). A source dataset used for validation provides its own privacy-preserving RNA factors to the system. Given the factors, the system returns a correction model to the source, where the source data is corrected. A target classifier without modification can then be validated on source-corrected data.

Corrections are learned using a regularized linear transformation between the data factors of source and target, which comprise of the PCA basis, gene-wise means, and gene-wise standard deviations of source and target, respectively, as depicted in FIG. 11B. The linear transformation is the solution of a non-convex objective function, which is optimized using an efficient computational approach based on projected gradient descent. Continuing the example of FIG. 11A discussed above, source and target factors are calculated as the principal components of RNA data. Next, the system learns a correction model from source to target eigenvectors (factors). Once the transformation has been learned, it can be applied on the held-out prospective source dataset for correction, followed by application of the target-trained classifier on the corrected source dataset, as depicted in FIG. 11C. Therefore, the learning module requires access only to the data factors of each dataset to learn the transformation for the source dataset.

Since the learning step of FIG. 11B is separated from the transform step of FIG. 11C, the transform step can be applied to new prospective data that was held-out in the learning step. The ability to transform data held-out from model training is deemed necessary for machine learning algorithms to avoid overfitting, by ensuring the test data for the predictor is not used for training. Including predictor test data in training can lead to information leakage and overly optimistic performance metrics. To avoid this, the evaluation data in transform step (transform) is kept independent of the train data in the learning step (fit). This fit-transform paradigm is extended by the present system and methods (“SpinAdapt”) to transcriptomic datasets.

### EXAMPLE 6

### Application to a Transcriptomic Dataset of Paired Patients

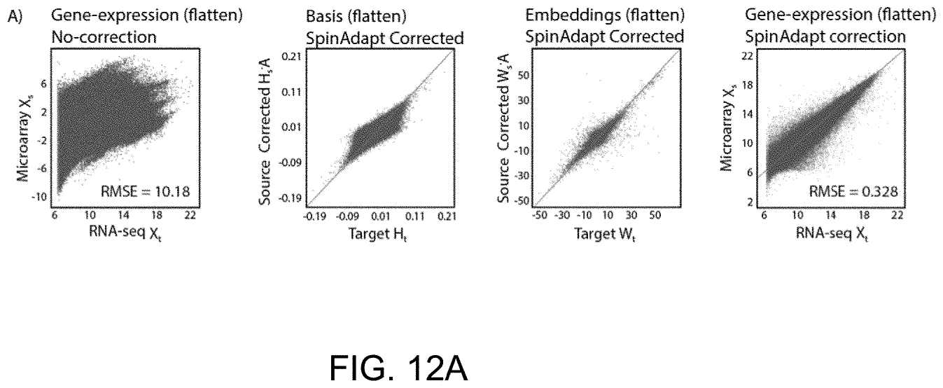

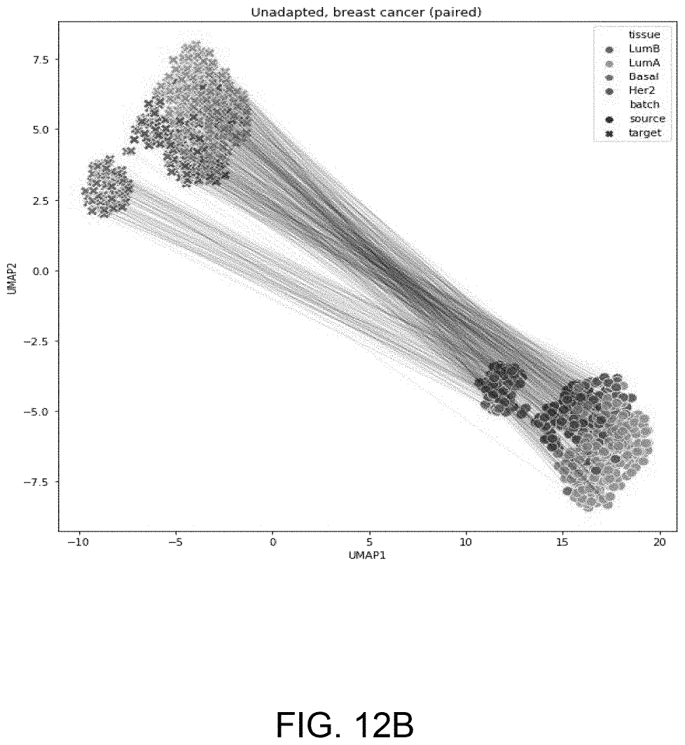

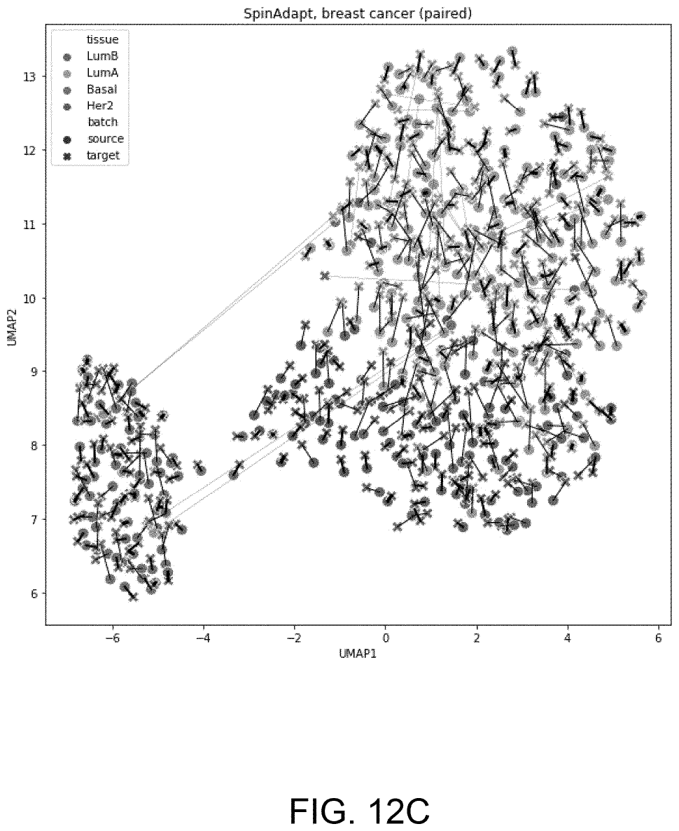

The training step of the algorithm is based on the idea of aligning the PCA basis of each dataset. To demonstrate the concept, SpinAdapt was applied on a transcriptomic dataset of paired patients, employing TCGA-BRCA cohort comprising 481 breast cancer patients, where RNA was profiled both with RNA-seg and microarray. The RNA-seq library was assigned as target and the microarray as source. The adaptation of source to target achieved alignment of the PCA basis and embeddings across datasets, which resulted in alignment of the paired expression datasets in the gene-expression space, as depicted in FIG. 12A.

The paired patients were composed of four cancer subtypes: Luminal A (LumA), Luminal B (LumB), Her2, and Basal. When the corrected source dataset is visualized with the target library in a two-dimensional space with UMAP, it was observed that each of the four subtypes were harmonized across the two libraries, as compared with before correction, as seen in FIGS. 12B and 12C. The subtype-wise homogenization is achieved without the use of subtype labels in the training step, demonstrating that alignment of basis in the PCA space achieves efficient removal of technical biases in the gene-expression space.

### Example 7

### Removal of Batch Effects

In another aspect, a simulation experiment was run to explore SpinAdapt for removal of batch effects, where a batch effect is simulated between synthetic datasets (source and target), and the source dataset is corrected using SpinAdapt.

In this regard, SpinAdapt is a batch correction method, which learns corrections between latent space representations that de-identify the sample-level information in each dataset. In this study, the PCA basis were chosen as the latent space representations for each dataset. SpinAdapt only required access to data factors of each dataset for computation and application of dataset corrections, where the data factors consist of the PCA basis, gene-wise means, and gene-wise variances of each dataset. Therefore, for the transfer of an RNA model from a training dataset to a validation dataset, only the data factors of the training dataset needed to be transferred along the RNA model, thereby maintaining ownership of the training dataset.

This example shows that the data factors are privacy-preserving, since an expression dataset cannot be recovered from the PCA basis of the dataset. In particular, in this example, let X be an expression matrix of size {genes×samples}, let the columns of a matrix W contain the eigenvectors of XX^{T}, let the columns of a matrix V contain the eigenvectors of X^{T}X, and let Σ be a diagonal matrix such that the entries are square roots of the eigenvalues of XX^{T}. Note that W and V are PCA basis for the columns and rows of the expression matrix X, respectively. An SVD decomposition of the expression matrix X can be written as X=WΣV^{T}, which implies W=X VΣ⁻¹, since the columns of matrix V are orthonormal and Σ is a diagonal matrix. Let H be another matrix H=VΣ⁻¹. Then, W=X VΣ⁻¹ can be rewritten as W=X H. From W=X H, it can be seen that the PCA basis matrix W is obtained via an affine transformation H, which de-identifies the original expression matrix X. Such affine transformations are called matrix masks in the privacy literature¹⁴. Since the matrix mask H is kept private, while only the PCA basis matrix W is made public, recovering original sample-level data X from W involves solving a highly underdetermined system¹⁵. Hence, the affine transformation H de-identifies the original data X, and the PCA factors W are privacy-preserving representations of the expression data X.

For this simulation, we set p=1000 and n=500, where p is number of genes and n is number of patients. To evaluate SpinAdapt, we simulated n source samples (denoted as Xₛ) from a p-dimensional multivariate normal distribution with mean μₛ and covariance matrix Σₛ:=CC′, where μₛ is a 1000-dimensional vector of uniform(0,10) random variables, C′ is the transpose of the matrix C, and C is a p-by-p matrix of standard normal random variables. To model the dataset bias, we generated a p-by-p matrix B, which is equal to the sum of an identity matrix and a p-by-p matrix of standard normal random variables. We generated target data Xₜ using Xₜ=XₛB+ϵₜ, where ϵₜ is a n-by-n matrix of normal random variables. The target data Xₜ represents n instances of a p-dimensional multivariate normal distribution with mean μₛBand covariance matrix Σₜ=B′ΣₛB+0.25/₁₀₀₀.

SpinAdapt then was trained on the PCA basis of Xs and Xt, which are Hs and Ht, respectively:

$${W}_{s} , {H}_{s} = P C A ( {X}_{s} ) \\ {W}_{t} , {H}_{t} = P C A ( {X}_{t} ) .$$

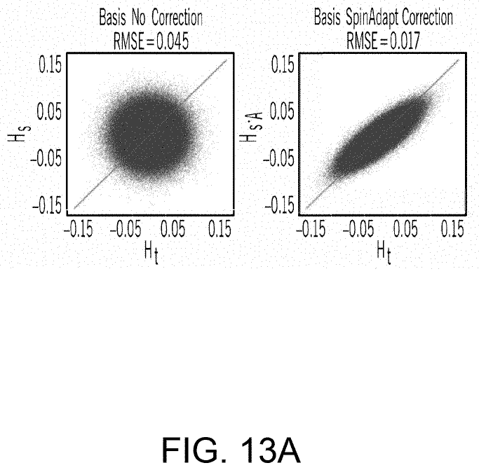

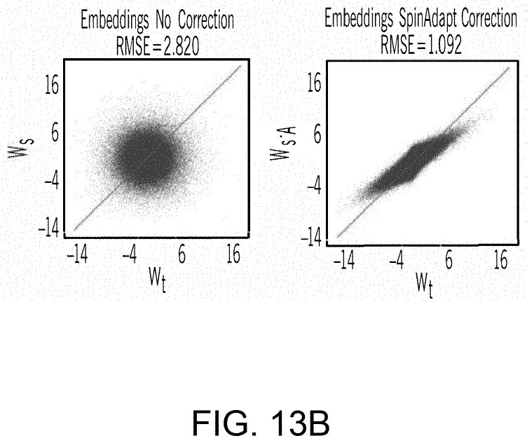

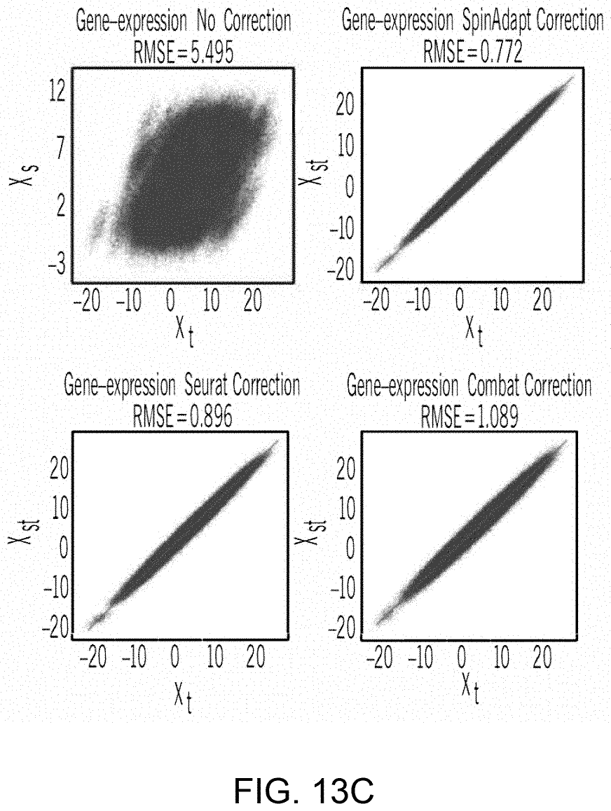

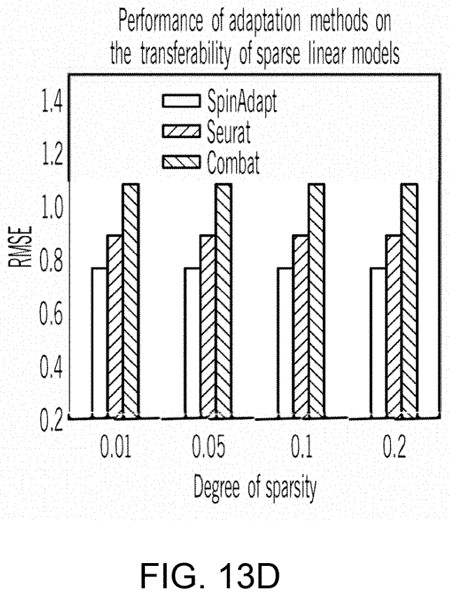

The basis correction (A is a linear basis change operator for SpinAdapt in this setting) to Hs approximates Ht with low error (RMSE=0.017). This is illustrated in FIG. 13A in the comparison of the scatter plot of the target basis and uncorrected source basis (RMSE=0.045) with the scatter pot of the target basis and the learned SpinAdapt corrected source basis (RSME=0.017). The source embeddings are corrected through Ws*A, which estimates Wt with RMSE=1.092. This is illustrated in FIG. 13B in the comparison of the scatter plot of the target embedding and uncorrected source embedding (RMSE=2.82) with the scatter pot of the target embedding and the SpinAdapt corrected source embedding (RSME=1.092).

The performance of SpinAdapt on correction of the simulated biased source dataset Xₛ was evaluated, drawing comparisons with no correction, Seurat, and Combat. To compare the correction performance for each method, we computed the RMSE for each technique. All of the methods achieved lower error than no correction. For example, FIG. 13C depicts this in scatter plots of paired expression values in the target and: un-corrected source (RMSE=5.5), SpinAdapt corrected source (RMSE=0.772), Seurat corrected source for Seurat (RMSE=0.896), and Combat corrected source (RMSE=1.089). As can be seen, SpinAdapt outperformed both Seurat and Combat.

In order to test the transferability of an arbitrary function across target and source corrected data, we generated 500 sparse linear models, each consisting of a normalized p-dimensional coefficient vector of standard normal random variables. We repeat the experiment for sparsity levels of 1%, 5%, 10%, and 20%, such that 10, 50, 100, and 200 components of the coefficient vectors have non-zero elements, respectively. For each sparsity level, we denote this collection of models as {β^{(m)}}ₘ₌₁⁵⁰⁰.

For each sparsity level, we calculated the RMSE of the differences between observed target values (yᵢ^{(m)}=xₜ_{ᵢ}β^{(m)}) and predicted values for the corrected source data (ŷᵢ^{(m)}=xₛₜ_{ᵢ}β^{(m)}), where xₛₜ_{ᵢ} represents source corrected dataset, the results of which are shown in FIG. 13D. As can be seen, SpinAdapt outperforms Seurat and ComBat in terms of RMSE, for any chosen level of sparsity.

### EXAMPLE 8

### Transfer of Diagnostic Predictors

We demonstrate the transfer of multiple distinct tumor subtype classifiers on four pairs of publicly available cancer datasets (bladder, breast, colorectal, pancreatic), covering 4,076 samples and three technological platforms (RNA-Seq, Affymetrix U133plus2 Microarray, and Human Exon 1.0 ST Microarray). Information about the datasets used is depicted in the following table:

TABLE 1

| Cancer type | | Source | | Target | |
|---|---|---|---|---|---|
| Bladder | Dataset | Seiler | | TCGA | |
| cancer | Assay | Affy Human- | | RNA-Seq | |
| | platform | Exon 1.0 ST | | | |
| | Subtype | Samples | % | Samples | % |
| | Ba-Sq | 114 | 38% | 116 | 40% |
| | LumP | 76 | 25% | 88 | 31% |
| | LUMU | 69 | 23% | 37 | 13% |
| | Stroma-rich | 24 | 8% | 31 | 11% |
| | LumNS | 18 | 6% | 16 | 6% |
| | Total | 301 | 100% | 288 | 100% |
| Colorectal | Dataset | G5E14333 | | TCGA | |
| cancer | Assay | Afymetrix hgu- | | RNA-Seq | |
| | platform | 133plus2 | | | |
| | Subtype | Samples | % | Samples | % |
| | CMS1 | 21 | 16% | 62 | 19% |
| | CM52 | 55 | 43% | 119 | 37% |
| | CM53 | 21 | 16% | 50 | 16% |
| | CM54 | 31 | 24% | 89 | 28% |
| | Total | 128 | 100% | 320 | 100% |
| Breast | Dataset | TCGA | | SCAN-B | |
| cancer | Assay | RNA-Seq | | RNA-Seq | |
| | platform | | | | |
| | Subtype | Samples | % | Samples | % |
| | Basal | 166 | 18% | 206 | 11% |
| | Her2 | 73 | 8% | 207 | 11% |
| | LumA | 470 | 52% | 1059 | 55% |
| | LumB | 192 | 21% | 466 | 24% |
| | Total | 901 | 100% | 1938 | 100% |
| Pancreatic | Dataset | TOGA | | Bailey | |
| cancer | Assay | RNA-Seq | | RNA-Seq | |
| | platform | | | | |
| | Subtype | Samples | % | Samples | % |
| | ADEX | 33 | 27% | 9 | 13% |
| | Immunogenic | 25 | 21% | 20 | 29% |
| | Progenitor | 42 | 35% | 21 | 30% |
| | Squamous | 21 | 17% | 20 | 29% |
| | Total | 121 | 100% | 70 | 100% |

Gene expression dataset pairs are generated across various cancer types (bladder, breast, colorectal, pancreatic) pertaining to microarray platforms and RNA-sequencing. Bladder cancer datasets pertain to Seiler and TCGA cohorts, which are downloaded from GSE87304 and ICGC under the identifier BLCA-US, respectively. Colorectal cancer datasets are downloaded from GSE14333 and ICGC under the identifier COAD-US, which are subsets of cohorts A and C, respectively, described in the ColoType prediction. Breast cancer datasets pertain to TCGA and SCAN-B cohorts, where the former is downloaded from ICGC under the identifier BRCA-US and the latter is obtained from GSE60789. Pancreatic cancer datasets are generated using the Bailey and TCGA cohorts, which are downloaded from ICGC under the identifiers PACA-AU and PAAD-US, respectively.

The subtype labels for patients across various cancer types were generated using well-accepted subtype annotations. Bladder cancer subtypes are labeled as luminal papillary (LumP), luminal nonspecified (LumNS), luminal unstable (LumU), stroma-rich, basal/squamous (Ba/Sq), and neuroendocrine-like (NE-like), generated using a consensus subtyping approach. Breast cancer subtypes are labeled as Luminal A (LumA), Luminal B (LumB), HER2-enriched (Her2), and Basal-like (Basal). Colorectal cancer subtypes are labeled as CMS1, CMS2, CMS3, CMS4, as published by Colorectal Cancer Subtyping Consortium (CRCSC). Pancreatic cancer subtypes are generated using expression analysis, labeled as squamous, pancreatic progenitor, immunogenic, and aberrantly differentiated endocrine exocrine (ADEX).

Once obtained, the data then was preprocessed. For each cancer type, we only kept patients with both expression data and subtype annotation labels available. Molecular subtypes with less than 5 patients in any cancer cohort were removed. For the microarray expression datasets (GSE14333 and Seiler, see Table 1), multiple probe sets may map to the same gene. The expression values were averaged across such probe sets to get gene expression value. Furthermore, for each cancer type, we removed genes with zero variance, and we only kept genes common between source and target, sorted in alphabetical order. Finally, we normalized the RNA-Seq datasets using the variance stabilizing transform (VST) from DeSeq2, whereas microarray data were not normalized beyond their publication.

Cumulatively, we validated the transfer of seventeen tumor subtypes across the four experiments, drawing comparisons of SpinAdapt with other batch correction methods like Com Bat and Seurat. For each dataset pair and tumor subtype, we trained a one-vs-rest tumor subtype classifier on the target dataset. The hyperparameters for each subtype classifier were chosen in a cross-validation experiment on the target dataset, while the source dataset was held-out from classifier training.

The following benchmarking methods were applied to the various batch correction methods being compared to SpinAdapt. For Seurat, we used the default package parameters, except when the number of samples in either dataset was less than 200, where the default value of k.filter does not work. Therefore, when the number of samples in either dataset was less than 200, we set the k.filter parameter to 50. For ComBat, a design matrix was created using the batch labels, and the method was implemented using the sva package version 3.34.0. Similarly, Limma was implemented using limma package version 3.42.2. For Seurat and Scanorama, we deployed package versions 3.2.2 and 1.6, respectively.

For SpinAdapt, we set the following parameters. For any given pair of source and target datasets, let p be the number of genes, nₛ be the number of samples in source, and nₜ be the number of samples in target. Across all experiments performed in this study, the parameters in SpinAdapt (Algorithm 1) are set as follows: α=0.01, λ=(2/3)*min(nₛ,nₜ), and varianceₙₒᵣₘis set to True when the source is microarray but False otherwise. However, for integration analysis of source and target datasets, we always set varianceₙₒᵣₘ=True.

In order to evaluate the various methods for transfer of diagnostic models, for each of the 17 cancer subtypes depicted in Table 1, we trained a one-vs-rest random forest classifier on the target dataset, such that the classifier learned to discriminate the selected subtype against all other subtypes in the target. Specifically, all target samples annotated with the selected subtype were given a positive label, while the rest of the target samples were assigned a negative label. The hyperparameters for the random forest classifier were learned in a three-fold cross-validation experiment on the target dataset.

We then compared multiple batch correction (adaptation) methods for transfer of the classifiers from target to the source dataset. The transfer requires adaptation of the source dataset to the target reference. For unbiased performance evaluation of a batch correction method, the test set for the classifier and the training set for the correction method need to be disjoint, so that the correction model does not train on the classifier test set. Thus, we proposed a framework for validating transfer of classifiers across datasets that avoids such information leakage.

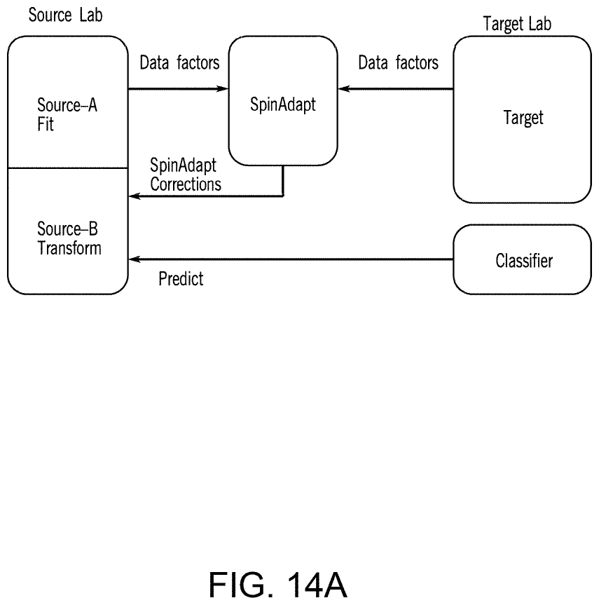

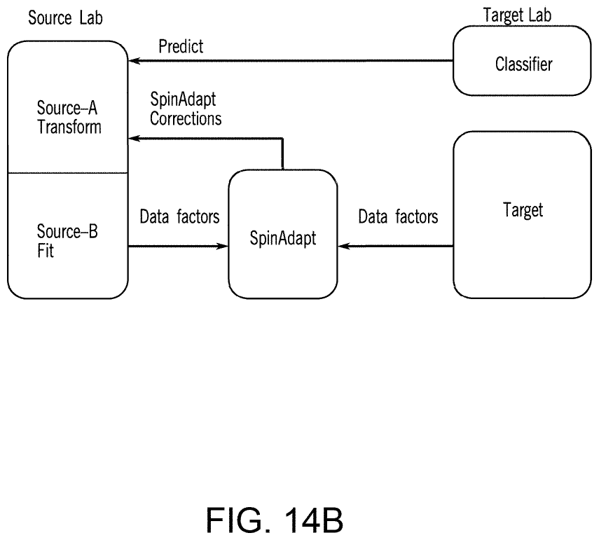

The validation framework randomly split the source dataset into two mutually-exclusive subsets: source-A and source-B. First, the adaptation model was trained from source-A to target (fit), and applied to source-B (transform). This involved fitting the adaptation model between source-A and target and transforming source-B using the adaptation model, followed by prediction on transformed Source-B using the target-trained classifier. The target classifier generated predictions on corrected source-B, as seen in FIG. 14A.

Second, the adaptation model was fit from source-B to target, followed by transformation of source-A and generation of predictions on corrected source-A, as seen in FIG. 14B. This involved fitting the adaptation model between source-B and target, followed by transformation and prediction on source-A. Under this framework, the correction model may never train on the test data for the predictor.

Finally, the classification performance was quantified by computing F-1 scores for all samples in the held-out corrected source-A and source-B subsets. In this example, we concatenated the held-out predictions on source-A and source-B, followed by performance evaluation using F-1 score. We repeated the entire procedure 30 times, choosing a different partitioning of source into source-A and source-B, and we reported the mean F-1 score for each subtype over the 30 iterations.

SpinAdapt's performance was evaluated using this framework, so the test set is always held-out from all training modules. Existing correction methods, like ComBat and Seurat, have currently not implemented a transformation method for out-of-sample data that is held-out from their training. Therefore, these methods had to be trained on the classifier test set in the aforementioned framework, followed by application of target-trained classifier on the corrected test set. Specifically, for Com Bat and Seurat, we fit-transformed source-A to target, fit-transformed source-B to target, and computed F-1 scores for all samples in the transformed source-A and source-B subsets. As before, we repeated the procedure 30 times and reported the mean F-1 score for each tumor subtype, using the same data splits as used for SpinAdapt validation, which enabled pairwise performance comparisons between SpinAdapt, Seurat, and ComBat.

Table 2 provides average (mean) F-1 scores for tumor subtype production, with std. error.

TABLE 2

| Cancer type | Subtype | SpinAdapt | Seurat | ComBat | Limma | Scanorama |
|---|---|---|---|---|---|---|
| Bladder | Ba-Sq | 0.98 (8e−4) | 0.90 (5e−3) | 0.94 (1e−3) | X | X |
| cancer | LumNS | 0.53 (9e−3) | 0.37 (2e−2) | 0.27 (9e−3) | X | X |
| | LumP | 0.74 (3e−3) | 0.65 (6e−3) | 0.62 (3e−3) | X | X |
| | LumU | 0.66 (4e−3) | 0.63 (1e−2) | 0.37 (4e−3) | X | X |
| | Stroma-rich | 0.79 (7e−3) | 0.44 (2e−2) | 0.24 (1e−2) | X | X |
| Colorectal | CMS-1 | 0.78 (5e−3) | 0.70 (8e−3) | 0.44 (6e−3) | X | X |
| cancer | CMS-2 | 0.93 (2e−3) | 0.89 (3e−3) | 0.83 (4e−3) | X | X |
| | CMS-3 | 0.85 (6e−3) | 0.45 (1e−2) | 0.26 (8e−3) | X | X |
| | CMS-4 | 0.86 (3e−3) | 0.87 (6e−3) | 0.84 (3e−3) | X | X |
| Breaset | Her2 | 0.78 (3e−3) | 0.76 (4e−3) | 0.68 (2e−3) | X | X |
| cancer | LumA | 0.91 (6e−4) | 0.86 (1e−3) | 0.91 (5e−4) | X | X |
| | LumB | 0.77 (2e−3) | 0.72 (2e−3) | 0.76 (1e−3) | X | X |
| | Basal | 0.99 (5e−4) | 0.98 (7e−4) | 0.97 (6e−4) | X | X |
| Pancreatic | ADEX | 0.81 (3e−3) | 0.49 (1e−2) | 0.54 (6e−3) | X | X |
| cancer | Progenitor | 0.86 (3e−3) | 0.65 (7e−3) | 0.69 (4e−3) | X | X |
| | Immunogenic | 0.64 (7e−3) | 0.40 (7e−3) | 0.49 (8e−3) | X | X |
| | Squamous | 0.72 (5e−3) | 0.72 (6e−3) | 0.76 (4e−3) | X | X |

For each subtype, we performed the two-sided paired McNemar test to identify if the differences between any pair of adaptation methods was statistically significant. Due to the rarity of positives for a selected subtype in each dataset, we performed the McNemar test only on samples with positive ground truth. Each positive sample was assigned a correct or incorrect classification label. Then, for each pair of correction methods, the McNemar test statistic was evaluated on the disagreements between correction methods on the positive samples. Table 3 lists p-values for comparing prediction performance between methods for tumor subtype prediction, reported as median over 30 experiments of the validation framework.

TABLE 3

| | | SpinAdapt- | SpinAdapt- | Seurat- |
|---|---|---|---|---|
| Cancer type | Subtype | Seurat | ComBat | ComBat |
| Bladder | Ba-Sq | 9.90E−05 | 1.60E−02 | 3.60E−02 |
| cancer | LumNS | 2.50E−01 | 3.90E−02 | 4.50E−01 |
| | LumP | 4.40E−01 | 4.80E−07 | 8.80E−07 |
| | LumU | 4.20E−01 | 1.90E−06 | 1.10E−05 |
| | Stroma-rich | 2.00E−03 | 1.20E−04 | 2.20E−01 |
| Colorectal | CMS-1 | 2.30E−01 | 3.90E−03 | 3.10E−02 |
| cancer | CMS-2 | 4.50E−01 | 3.90E−03 | 9.40E−02 |
| | CMS-3 | 2.70E−03 | 1.20E−04 | 2.50E−01 |
| | CMS-4 | 1.70E−01 | 6.20E−02 | 6.20E−01 |
| Breast | Her2 | 5.50E−01 | 3.70E−04 | 1.70E−02 |
| cancer | LumA | 1.30E−12 | 7.40E−01 | 3.50E−12 |
| | LumB | 6.70E−01 | 2.10E−01 | 2.20E−01 |
| | Basal | 5.00E−01 | 7.00E−02 | 2.20E−01 |
| Pancreatic | ADEX | 6.10E−05 | 2.40E−04 | 8.80E−01 |
| cancer | Progenitor | 3.40E−03 | 2.00E−03 | 6.20E−01 |
| | Immunogenic | 3.70E−03 | 1.60E−02 | 6.20E−01 |
| | Squamous | 5.00E−01 | 2.50E−01 | 1.00E+00 |

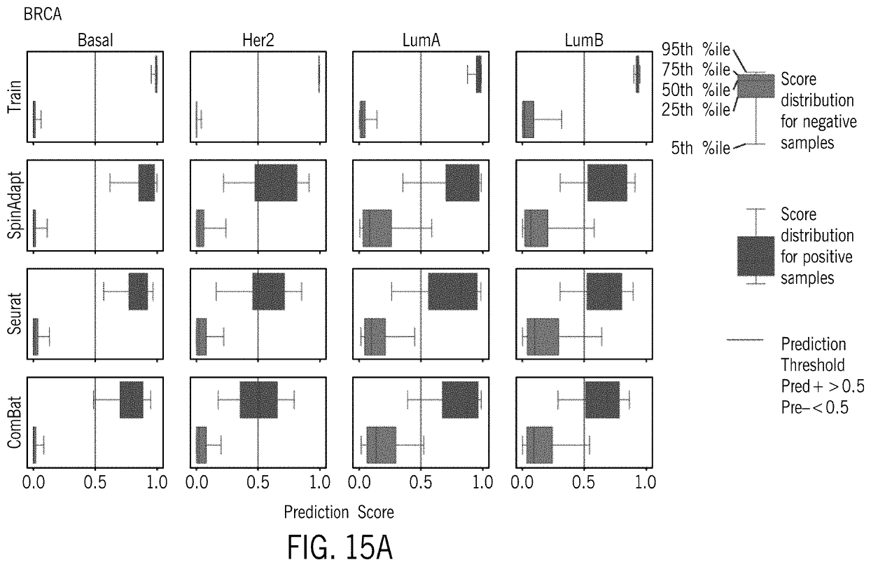

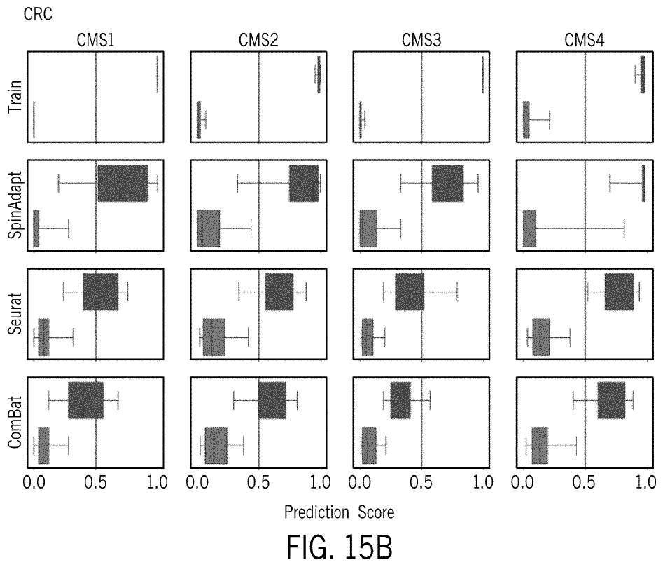

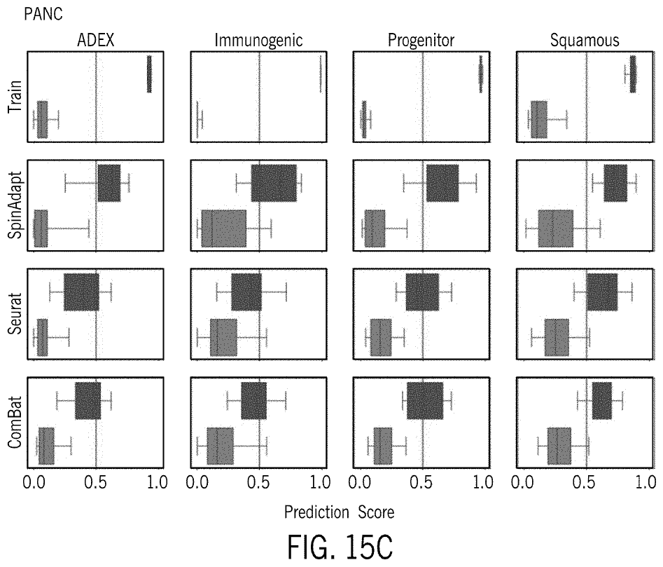

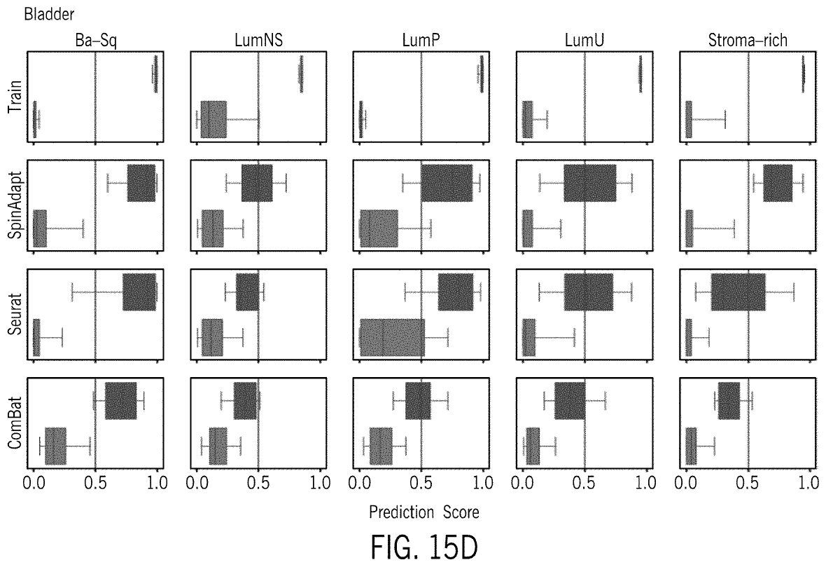

FIGS. 15A-D provide boxplots for predictor scores on batch-corrected source dataset for each cancer subtype. For each cancer subtype (column) and correction method (row), the boxplots for positive and control samples are plotted separately, and the vertical line represents the decision threshold at 0.5. Better performance is achieved when control and positive sample score distributions are shifted to the left and right, respectively. In FIG. 15A, for breast cancer subtypes: SpinAdapt obtains higher median test scores on the positive samples. In FIG. 15B, for colorectal cancer subtypes: the SpinAdapt test score distributions for positive samples are shifted to the right, but CMS4 subtype suffers from lower specificity. In FIG. 15C, for pancreatic cancer subtypes: the SpinAdapt test score distributions for positive samples are shifted to the right, compared with Seurat and ComBat. In FIG. 15D, for bladder cancer subtypes: SpinAdapt obtains lower median test scores on the control samples for LumP subtype, while obtaining higher median test scores on the positive samples for the LumNS and Stroma-rich subtypes, compared with Seurat and ComBat.

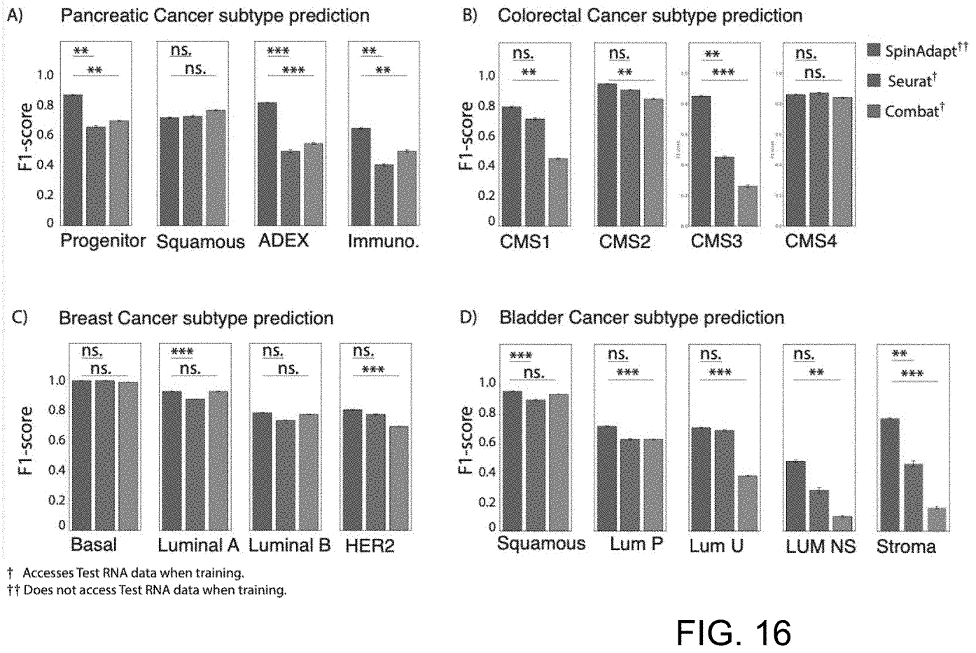

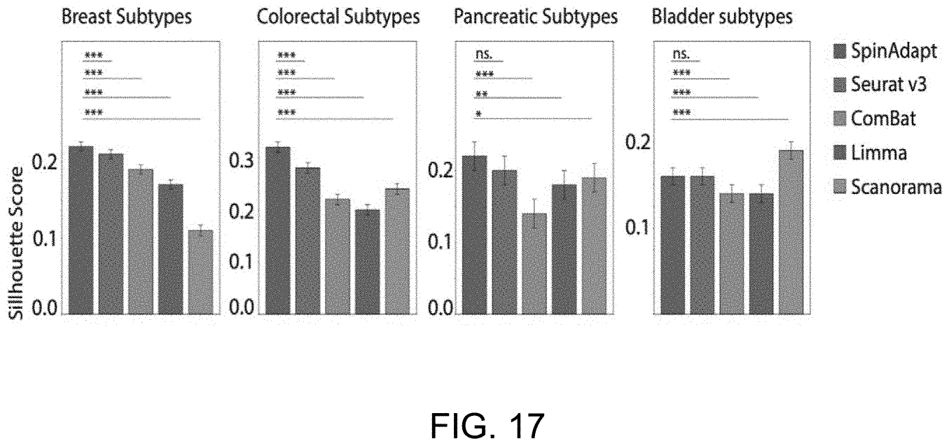

FIG. 16 illustrates subtype prediction performance on held-out source data. We trained subtype predictors on target data and evaluated them on source data. Source data is split into two disjoint subsets such that the correction model is trained on one subset and the predictor performance is evaluated on the other held-out subset. Seurat and Com Bat do not support a fit-transform paradigm, and therefore they require access to predictor held-out evaluation data to learn the correction model. For each subtype, the vertical bar represents the mean F-1 score and the error bar represents the standard error over 30 repetitions of the experiment. SpinAdapt either ties or outperforms Seurat and ComBat on: pancreatic cancer, colorectal cancer, breast cancer, and bladder cancer subtypes. Significance testing was done by a two-sided paired McNemar test, as discussed above.

With regard to integration of source data to target data, quantification of integration performance using silhouette score finds SpinAdapt to provide significantly better integrations in breast, colorectal, and pancreatic cancer datasets. On bladder cancer dataset, Scanorama significantly outperforms SpinAdapt, but SpinAdapt significantly outperforms Corn Bat and Limma. Significance testing in this regard is determined by two-sided paired Wilcoxon test. (ns: P≥0.05, *P<0.05, **P<0.01, ***P<0.001). Statistical significance is defined at P<0.05.

As these figures illustrate, SpinAdapt significantly outperformed Seurat on seven out of the seventeen tumor subtypes including Pancreatic subtypes: Progenitor, ADEX, Immunogenic, Colorectal subtypes: CMS3, Breast subtypes: Luminal A, Bladder subtypes: Squamous and Stroma. SpinAdapt also significantly outperformed ComBat on eleven out of the seventeen subtypes including Pancreatic subtypes: Progenitor, ADEX, Immunogenic, Colorectal subtypes: CMS1, CMS2, CMS3, Breast Subtypes: Her2, Bladder subtypes: Lump P, Lum U, Lum NS, and Stroma. SpinAdapt was not significantly outperformed by either Seurat or Corn Bat for any subtype.

### EXAMPLE 9

### Evaluation Methods for Dataset Integration

A common task for RNA-based algorithms is dataset integration (batch mixing). There is an inherent trade-off between batch mixing and preservation of the biological signal within integrated datasets. To quantify preservation of the biological signal, we quantified subtype-wise separability (no mixing of tumor subtypes) in the integrated datasets. Therefore, for high data integration performance, we wanted to minimize subtype mixing while maximizing batch mixing.

Dataset integration, an RNA-homogenization task that requires access to sample-level data, is commonly adopted for single-cell RNA homogenization. To evaluate the tradeoff between privacy preservation and full data access, we compared SpinAdapt to Seurat, Combat, Limma, and Scanorama for integration of bulk-RNA datasets in the four sets of experiments discussed previously (see Table 1). For high integration performance, we wanted to maximize dataset mixing while maintaining subtype-wise separability (no mixing of tumor subtypes) within integrated datasets.

To evaluate the various integration methods, we employed Uniform Manifold Approximation and Projection (UMAP) transform in conjunction with the average silhouette width (ASW) and local inverse Simpson's index (LISI) (methods). For each of the four cancer dataset pairs, the silhouette score was computed for each integrated sample in source and target, and then the average silhouette score was reported across all samples, as shown in FIG. 17 and in Table 4 below.

To compare SpinAdapt with the other batch integration methods, we assessed the goodness of batch mixing and tissue type separation. First, we employed the average silhouette width (ASW) to quantify batch mixing and tissue segregation. The silhouette score of a sample is obtained by subtracting the average distance to samples with the same tissue label from the average distance to samples in the nearest cluster with respect to the tissue label, and then dividing by the larger of the two values. Therefore, the silhouette score for a given sample varies between −1 and 1, such that a higher score implies a good fit among samples with the same tissue label, and vice versa. In other words, a higher average silhouette width implies mixing of batches within each tissue type or/and separation of samples from distinct tissue types.

To explicitly quantify batch mixing and tissue segregation, independently, we employed the local inverse Simpson's index (LISI). The LISI metric assigns a diversity score to each sample by computing the effective number of label types in the local neighborhood of the sample. Therefore, the notion of diversity depends on the label under consideration. When the label is set to batch membership, the resulting metric is referred to as batch LISI (bLISI), since it measures batch diversity in the neighborhood of each sample. Higher bLISI values indicate more homogenous mixing of the two datasets. When the label is set to tissue type, the resulting metric is referred to as tissue LISI (tLISI), since it measures tissue type diversity in sample neighborhood. For tLISI, lower values indicate that subtype clusters are more homogenous. For each integration method and cancer dataset, we report average (mean and std. error) bLISI and tLISI scores across all samples in source and target datasets in the following table. We also report a ratio of bLISI/tLISI, where higher values indicate more homogenous mixing of the two datasets and/or homogenous subtype clusters. Moreover, we report a silhouette score, where higher values indicate that subtype clusters are more homogenous.

TABLE 4

| | | SpinAdapt | Seurat | ComBat | Limma | Scanorama |
|---|---|---|---|---|---|---|
| Bladder | tLISI | 1.6 (3e−2) | 1.62 (3e−2) | 1.73 (3e−2) | 1.68 (3e−2) | 1.57 (3e−2) |
| cancer | bLISI | 1.85 (7e−3) | 1.88 (5e−3) | 1.60 (1e−2) | 1.68 (1e−2) | 1.74 (1e−2) |
| | bLISI/tLISI | 1.35 (2e−2) | 1.33 (2e−2) | 1.13 (2e−2) | 1.20 (2e−2) | 1.30 (2e−2) |
| | Silhouette | 0.16 (1e−2) | 0.16 (1e−2) | 0.14 (1e−2) | 0.14 (1e−2) | 0.19 (1e−2) |
| Colorectal | tLISI | 1.33 (2e−2) | 1.40 (2e−2) | 1.50 (3e−2) | 1.53 (3e−2) | 1.50 (3e−2) |
| cancer | bLISI | 1.66 (1e−2) | 1.63 (1e−2) | 1.43 (2e−2) | 1.48 (1e−2) | 1.58 (1e−2) |
| | bLISI/tLISI | 1.35 (2e−2) | 1.29 (2e−2) | 1.06 (2e−2) | 1.08 (2e−2) | 1.18 (2e−2) |
| | Silhouette | 0.32 (1e−2) | 0.28 (1e−2) | 0.22 (1e−2) | 0.20 (1e−2) | 0.24 (1e−2) |
| Breast | tLISI | 1.41 (9e−3) | 1.45 (1e−2) | 1.44 (9e−3) | 1.45 (9e−3) | 1.51 (1e−2) |
| cancer | bLISI | 1.63 (5e−3) | 1.68 (5e−3) | 1.59 (5e−3) | 1.62 (5e−3) | 1.46 (8e−3) |
| | bLISI/tLISI | 1.25 (7e−3) | 1.29 (8e−3) | 1.21 (7e−3) | 1.23 (8e−3) | 1.05 (7e−3) |
| | Silhouette | 0.22 (6e−3) | 0.21 (6e−3) | 0.19 (6e−3) | 0.17 (6e−3) | 0.11 (7e−3) |
| Pancreatic | tLISI | 1.74 (4e−2) | 1.85 (5e−2) | 1.96 (4e−2) | 1.85 (5e−2) | 1.83 (5e−2) |
| cancer | bLISI | 1.82 (1e−2) | 1.79 (2e−2) | 1.76 (2e−2) | 1.75 (2e−2) | 1.72 (2e−2) |
| | bLISI/tLISI | 1.12 (2e−2) | 1.06 (2e−2) | 0.97 (2e−2) | 1.03 (2e−2) | 1.02 (2e−2) |
| | Silhouette | 0.22 (2e−2) | 0.20 (2e−2) | 0.14 (2e−2) | 0.18 (2e−2) | 0.19 (2e−2) |

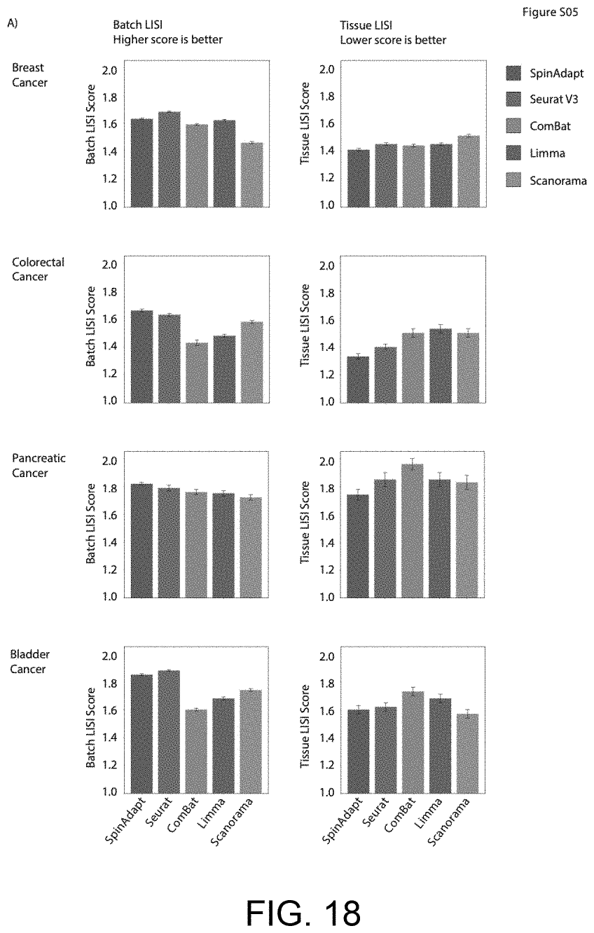

FIG. 18 illustrates quantification of dataset integration performance using batch LISI (bLISI) and tissue LISI (tLISI) metrics, where bLISI measures batch homogeneity and tLISI measures subtype heterogeneity in local sample neighborhoods. For each dataset, correction method, and performance metric, the associated barplot reports the mean and standard error over all the integrated samples.

The following table provides p-values for comparing integration performance between SpinAdapt and other methods.

TABLE 5

| | | Spin- | Spin- | Spin- | |
|---|---|---|---|---|---|
| | | Adapt- | Adapt- | Adapt- | SpinAdapt- |
| | | Seurat | ComBat | Limma | Scanorama |
| Bladder | tLISI | 4.50E−06 | 9.50E−10 | 2.30E−07 | 9.60E−01 |
| cancer | bLISI | 1.60E−02 | 9.00E−61 | 4.50E−34 | 1.50E−14 |
| | Silhouette | 6.50E−01 | 9.20E−06 | 3.60E−08 | 1.20E−05 |
| Colorectal | tLISI | 2.30E−07 | 5.70E−17 | 5.20E−19 | 2.90E−17 |
| cancer | bLISI | 1.10E−01 | 6.60E−31 | 1.10E−25 | 1.50E−06 |
| | Silhouette | 8.30E−05 | 4.50E−26 | 1.20E−26 | 2.90E−19 |
| Breast | tLISI | 9.10E−13 | 2.30E−15 | 8.70E−15 | 1.70E−42 |
| cancer | bLISI | 1.20E−30 | 4.00E−12 | 4.30E−01 | 5.20E−94 |
| | Silhouette | 2.00E−11 | 1.10E−129 | 1.30E−122 | 7.00E−95 |
| Pancreatic | tLISI | 2.20E−02 | 3.10E−10 | 1.60E−03 | 1.40E−02 |
| cancer | bLISI | 3.20E−02 | 3.70E−05 | 1.60E−04 | 8.90E−08 |
| | Silhouette | 1.30E−01 | 8.10E−06 | 7.30E−03 | 4.50E−02 |

Even though SpinAdapt did not have access to sample-level data when learning the transformation between source and target, it significantly outperformed each of the other methods for colorectal cancer and breast cancer (P<10e-5 and P<10e-34, respectively). For pancreatic cancer, SpinAdapt outperformed ComBat, Limma, and Scanorama (P<0.05 for each method). For bladder cancer, Scanorama outperformed SpinAdapt (P<10e-5), whereas SpinAdapt outperformed ComBat and Limma (P<10e-6). Even though the integration performance of SpinAdapt can be improved via direct access to samples, it significantly outperformed most of the competing methods in each experiment.

When comparing methods using average bLISI, which measures dataset mixing, Seurat outperforms SpinAdapt on breast and bladder cancer datasets (P<10e-3), whereas SpinAdapt outperforms ComBat, Limma, and Scanorama on colorectal and pancreatic cancer datasets (P<10e-3). As seen in Tables 4 and 5, when comparing methods using average tLISI, SpinAdapt significantly outperforms all other methods on breast (P<10e-13), colorectal (P<10e-7), and pancreatic (P<0.05) cancer datasets, implying SpinAdapt best preserves molecular structures for dataset integration. When comparing methods using tLISI on the bladder cancer dataset, SpinAdapt outperforms Seurat, ComBat, Limma (P<10e-6), whereas Scanorama outperforms SpinAdapt without significance.

The various integration metrics including silhouette, bLISI, and tLISI scores were computed on the UMAP embeddings of the integrated datasets for each cancer type listed in Table 1, above. Specifically, the scores in each experiment were computed on the first 50 components of the UMAP transform, where the UMAP embeddings were computed using default parameters of the package. The average silhouette width, bLISI, and tLISI scores are reported along with the standard errors in Table 4. For each metric provided in Table 5, significance testing between methods is performed by a two-sided paired Wilcoxon test.

### EXAMPLE 10

### Evaluation Methods for Transfer of Prognostic Models

For three of the four cancer datasets discussed above (Pancreatic, Colorectal, and Breast), we trained a Cox Proportional Hazards model on the target dataset using a gene signature determined through an ensemble method performed on the target dataset in order to demonstrate a transfer of Cox regression models across distinct datasets for those cancer types. Details of the datasets used in this example are shown in the following table.

TABLE 6

| Cancer type | | Source | Target |
|---|---|---|---|
| Colorectal | Dataset | GSE14333 | TCGA |
| cancer | Assay platform | Affymetrix hgu- | RNA-Seq |
| | | 133plus2 | |
| | Subtype | 226 | 579 |
| Breast | Dataset | TCGA | SCAN-B |
| cancer | Assay platform | RNA-Seq | RNA-Seq |
| | Subtype | 1038 | 2919 |
| Pancreatic | Dataset | PACA-AU | PACA-CA |
| cancer | Assay platform | Array-based gene | RNA-Seq |
| | | expression | |
| | Samples | 267 | 186 |

The risk thresholds for the survival model were determined based on the upper and lower quartiles of the distributions of log partial hazards of the target dataset, such that samples with a predicted log partial hazard higher than the 75% percentile of said distribution were predicted to be high risk, and samples with a predicted log partial hazard lower than the 25% percentile of said distribution were predicted to be low risk.

We then compared multiple batch correction (adaptation) methods for transfer of the prognostic models from target to the source dataset. For each cancer type, the source dataset was adapted to the target using SpinAdapt, Seurat, and ComBat. The target-trained Cox PH model was used to generate predictions (log partial hazards) on all samples from the source dataset, both for the uncorrected source dataset and the three correction methods. The risk thresholds determined on the target dataset were used to classify samples from the source dataset as either low risk, high risk, or unclassified, based on their predicted log partial hazards values.

The ensemble method for feature selection uses four ranked lists of genes, based on different statistical tests or machine learning models: Chi-square scores, F-scores, Random Forest importance metrics, and univariate Cox PH p-value. We tested the predictive values of various permutations of genes of increasing length (n=10, 50, 75, 100, 200, 300, 500 genes) as signatures of a Cox PH model trained and tested on random splits of the target dataset, in a five-fold cross-validation setting, where 50% of the target dataset was assigned to the training set and the remainder was assigned to the test set.

The performance of the prognostic models is quantified by computing the c-indices as well as the 5-year log-rank p-value and 5-year hazard ratio (HR) of the combined predicted high risk and low risk groups of source samples for each cancer type and each adaptation method.

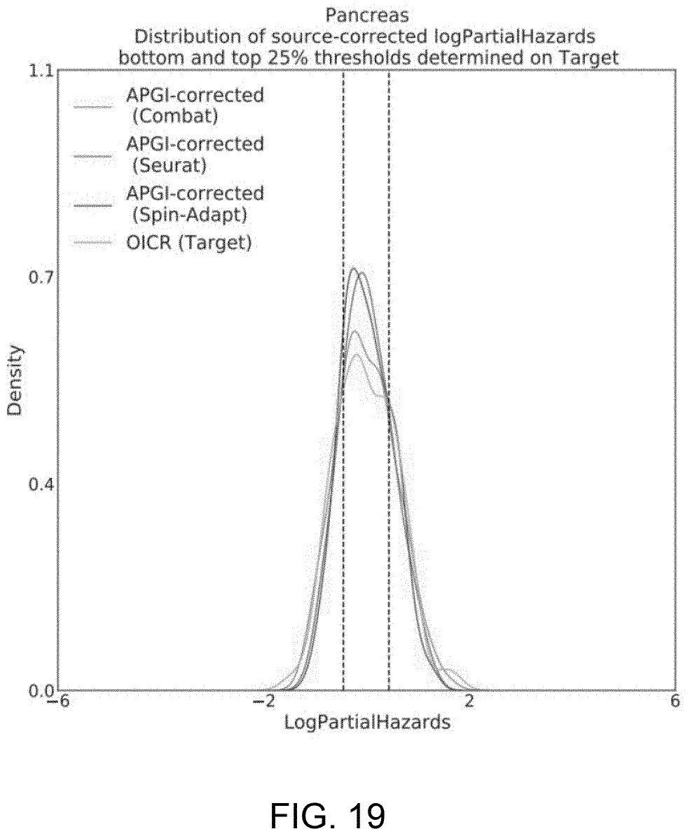

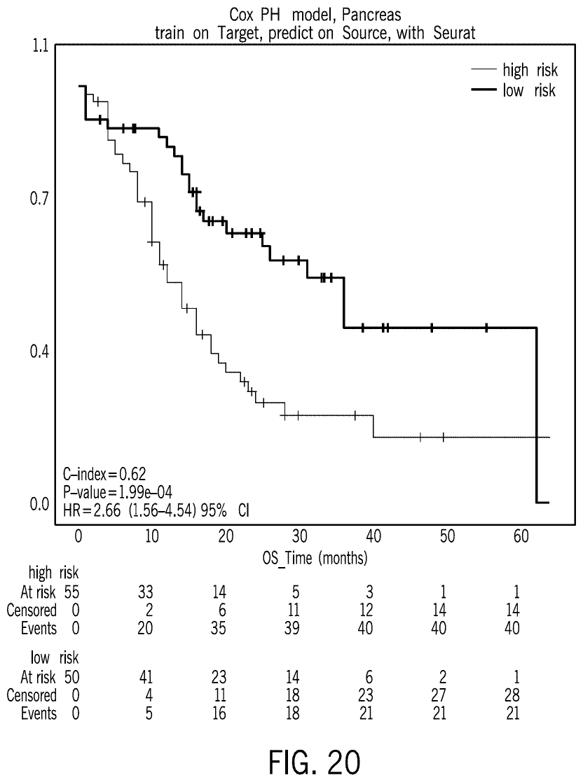

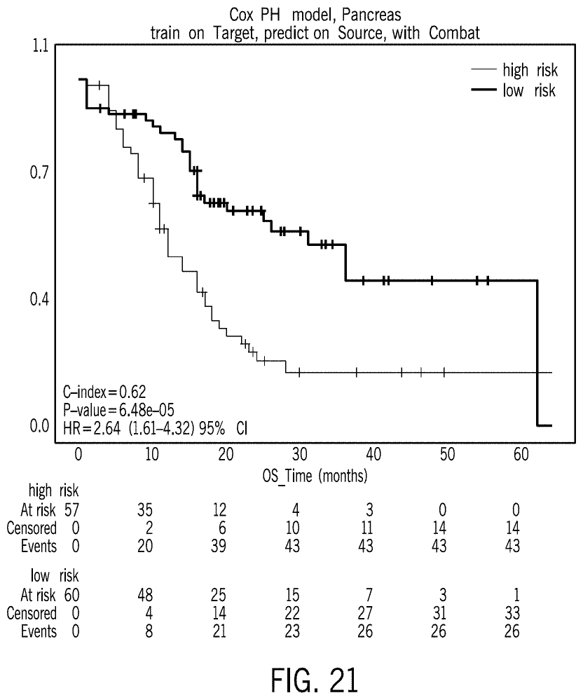

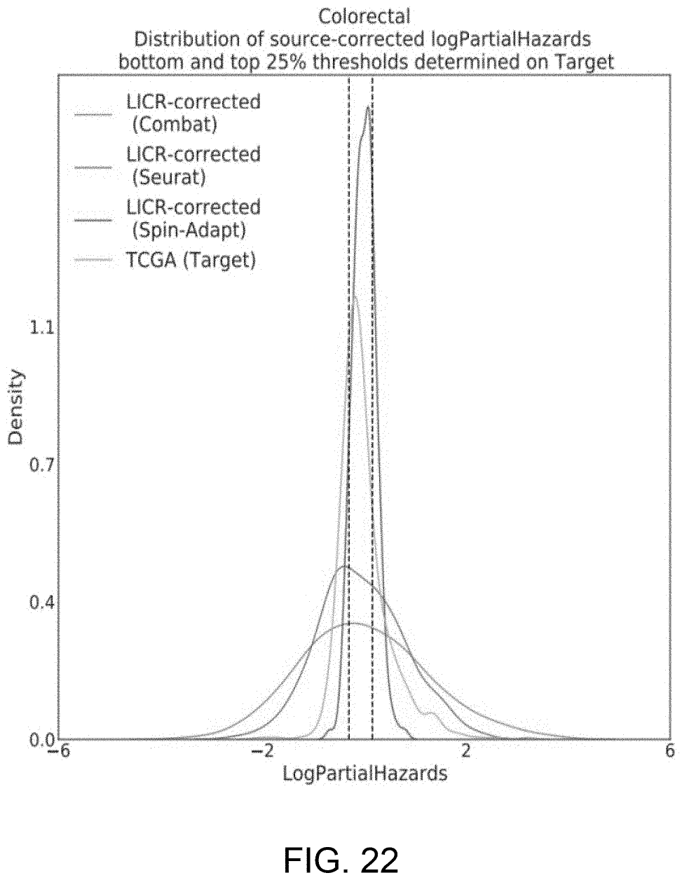

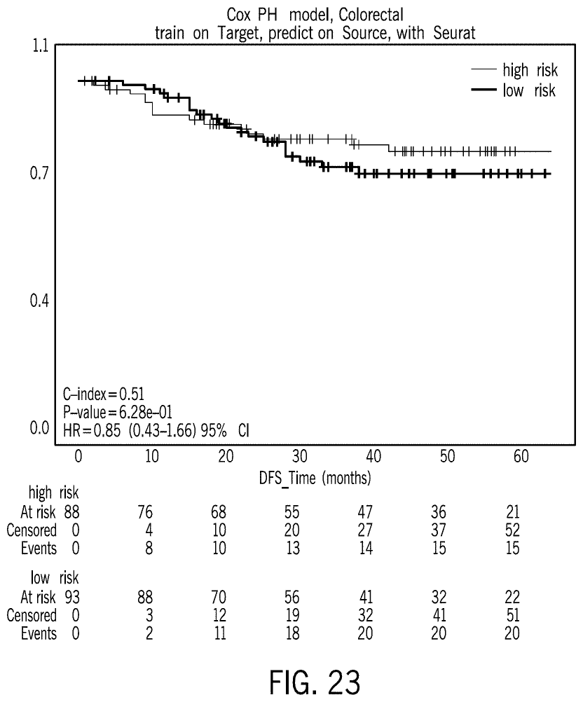

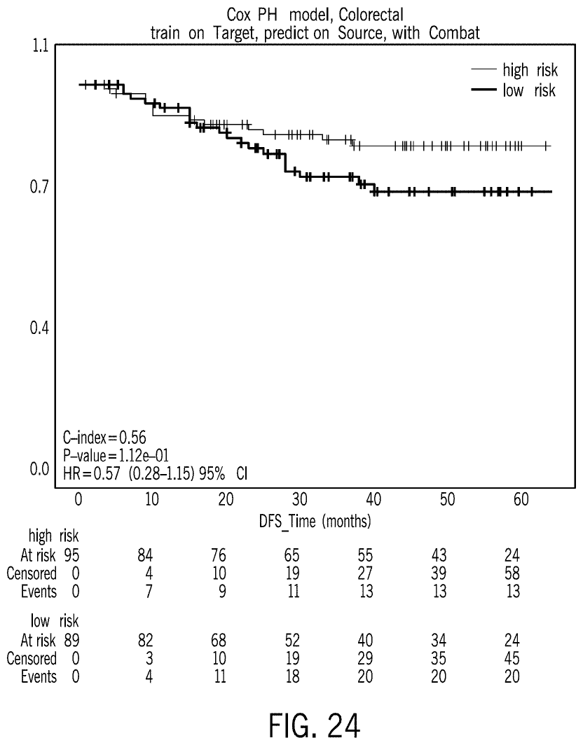

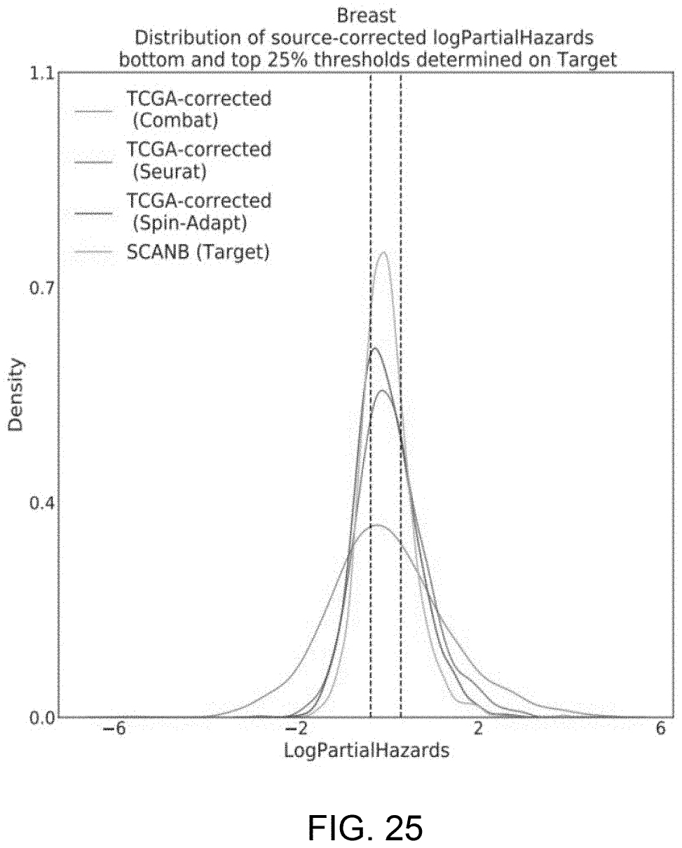

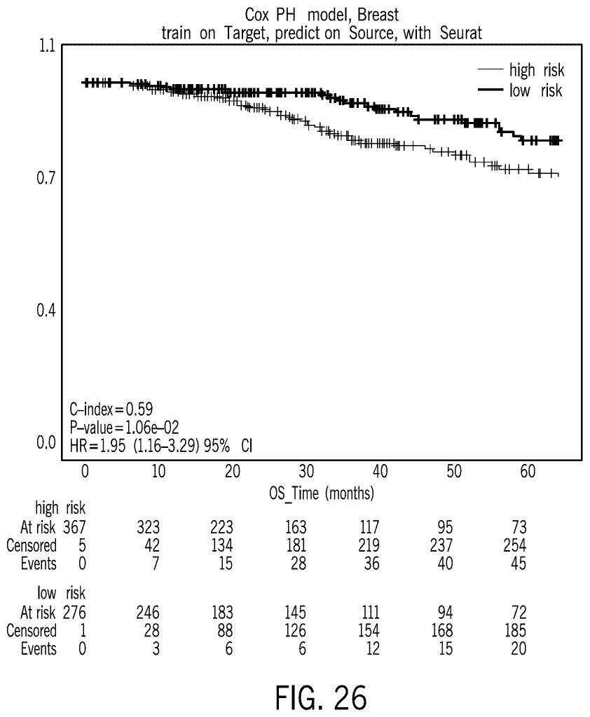

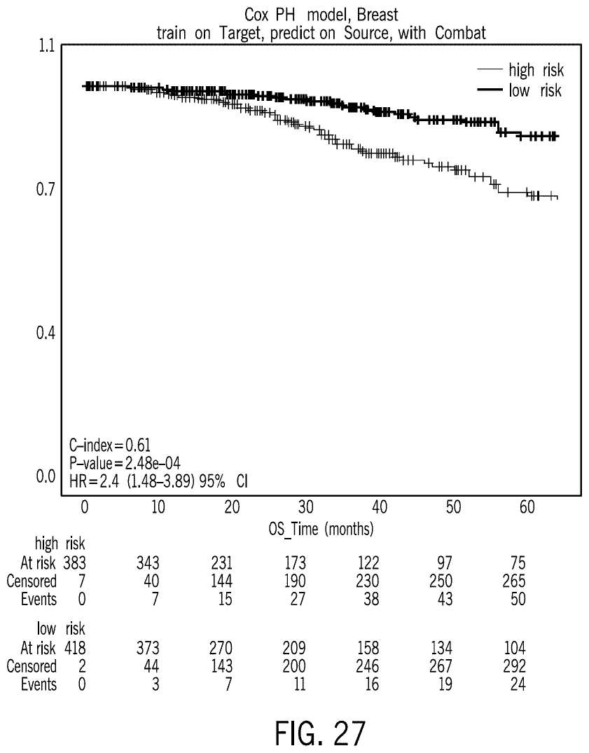

FIGS. 19-27 are distribution of log-partial hazards for the target-trained Cox model on the target dataset, source dataset, and corrected source dataset, for each of the three cancer types (breast, colorectal, pancreatic), or survival curves for predicted low-risk and high-risk groups on the validation dataset for each of the three cancer types, using Seurat and ComBat.

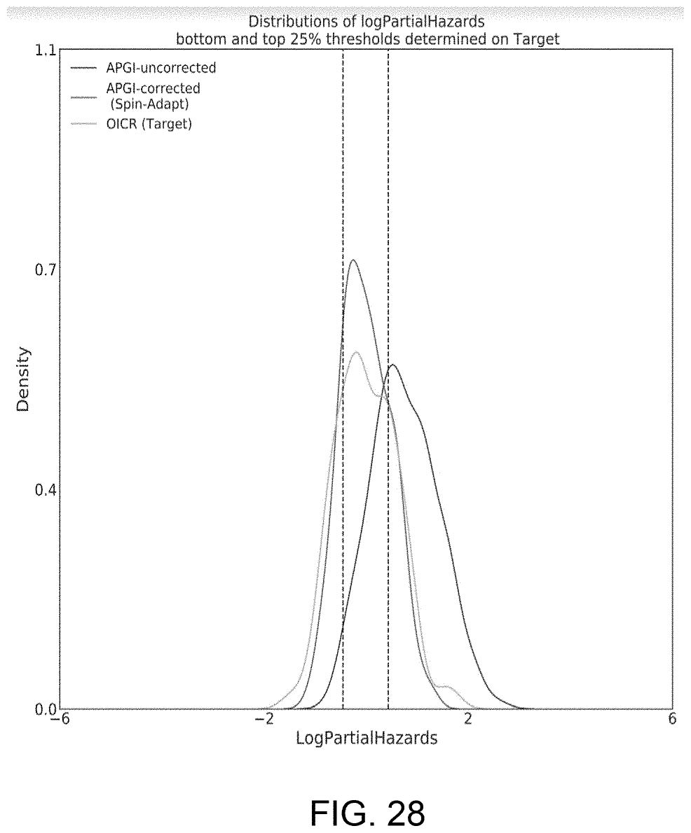

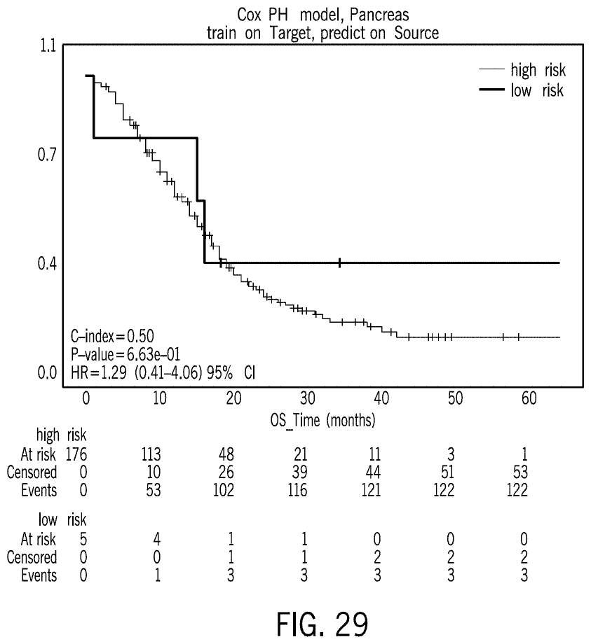

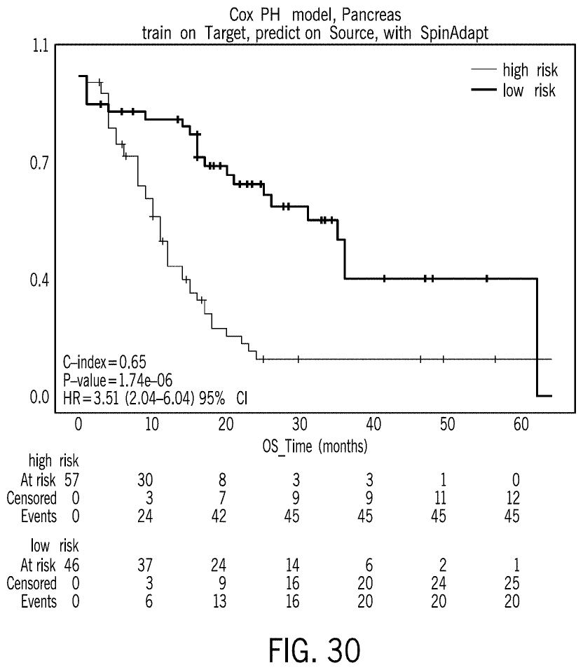

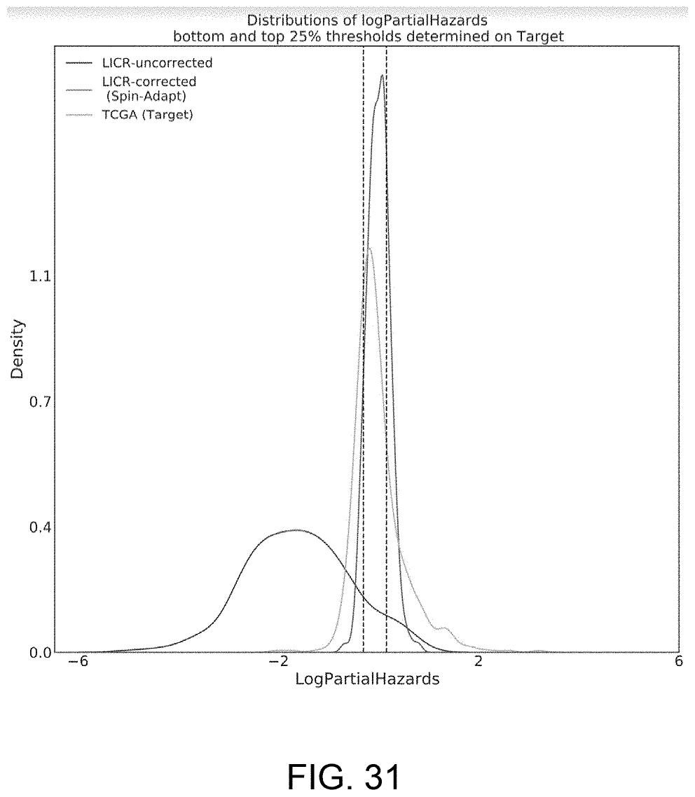

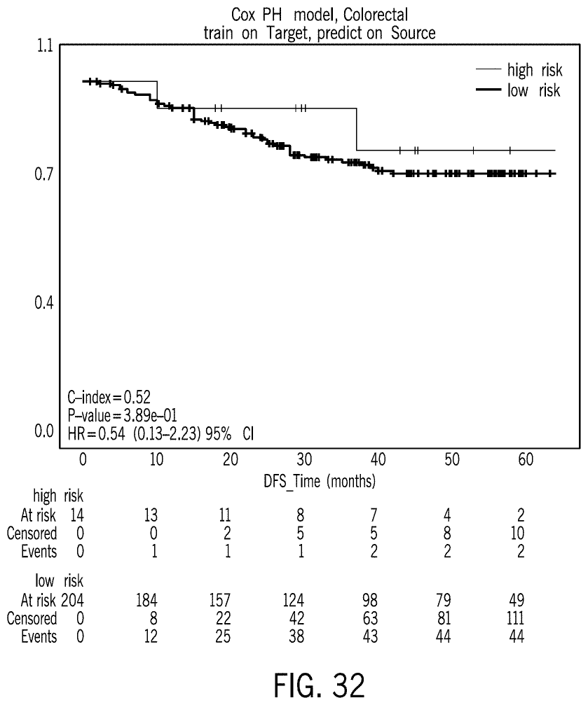

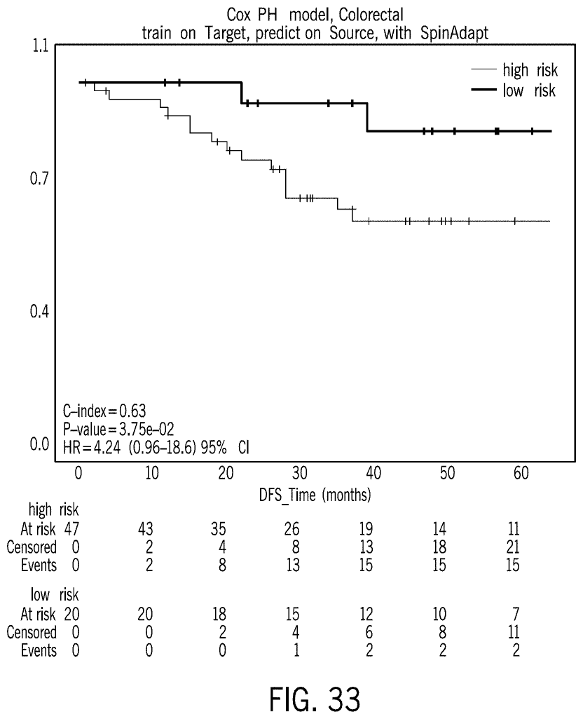

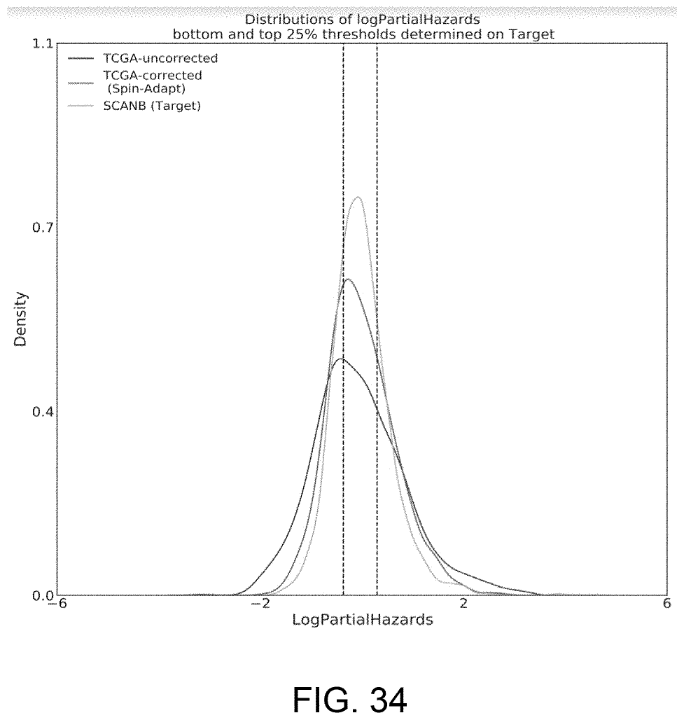

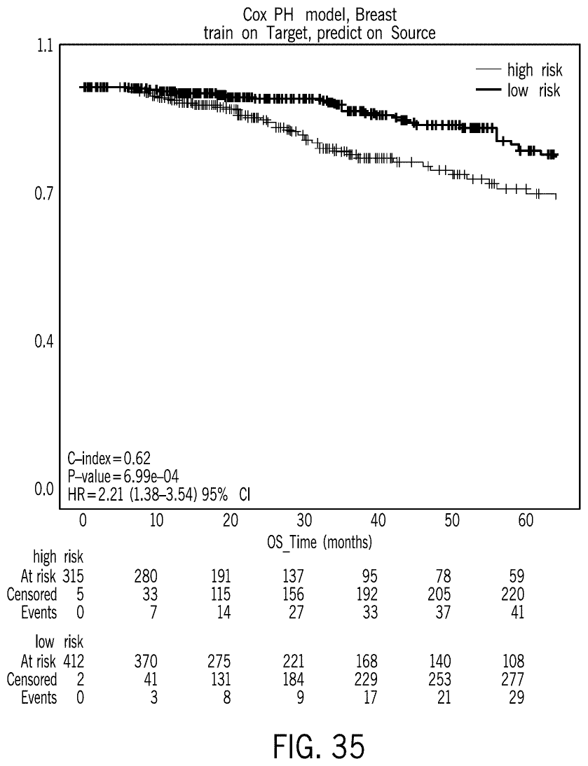

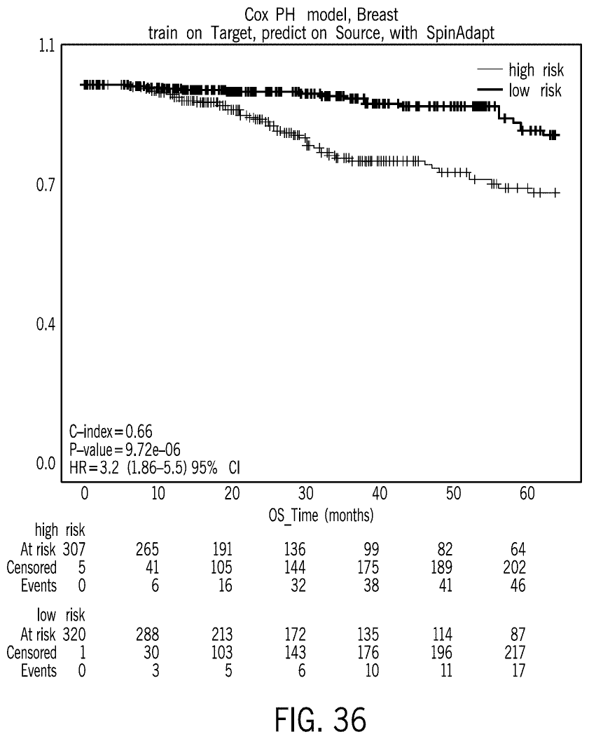

FIGS. 28-36 similarly are distribution of log-partial hazards for the target-trained Cox model on the target dataset, source dataset, and corrected source dataset, for each of the three cancer types, or survival curves for predicted low-risk and high-risk groups on the validation dataset for each of the three cancer types (breast, colorectal, pancreatic), but using SpinAdapt.

Similarly, the following table provides Concordance index (C-index), P value, and Hazard Ratio (HR) with 95% Confidence Interval values for comparing transfer of COX regression models using SpinAdapt, Seurat, and ComBat.

TABLE 7

| | | | | Hazard Ratio (HR) |
|---|---|---|---|---|
| | | C-index | P-value | (95% CI for HR) |
| Colorectal | SpinAdapt | 0.63 | 3.75e−02 | 4.24 (0.96-18.6) |
| cancer | Seurat | 0.51 | 6.28e−01 | 0.85 (0.43-1.66) |
| | ComBat | 0.56 | 1.12e−01 | 0.57 (0.28-1.15) |
| Breast | SpinAdapt | 0.66 | 9.72e−06 | 3.2 (1.86-5.50) |
| cancer | Seurat | 0.59 | 1.06e−02 | 1.95 (1.16-3.29) |
| | ComBat | 0.61 | 2.48e−04 | 2.4 (1.48-3.89) |
| Pancreatic | SpinAdapt | 0.65 | 1.74e−06 | 3.51 (2.04-6.04) |
| cancer | Seurat | 0.62 | 1.99e−04 | 2.66 (1.56-4.54) |
| | ComBat | 0.62 | 6.48e−05 | 2.64 (1.61-4.32) |

SpinAdapt demonstrates high survival prediction accuracy for all datasets (C-index, log-rank p-value, HR): colorectal [0.63, 4e-2, 4.24], breast [0.66, 1 e-5, 3.2], pancreatic [0.65, 2e-6, 3.51], in contrast with uncorrected source dataset: colorectal [0.52, 4e-1, 0.54], breast [0.62, 7e-4, 2.2], pancreatic [0.50, 7e-1, 1.29]. Furthermore, SpinAdapt outperforms Seurat: colorectal [0.51, 7e-1, 0.85], breast [0.59, 2e-2, 2.0], pancreatic [0.62, 2e-1, 2.66], as well as Com Bat: colorectal [0.56, 2e-1, 0.57], breast [0.61, 3e-4, 2.4], pancreatic [0.62, 7e-5, 2.64], in terms of all performance metrics for all cancer types.

The best performing signature was determined based on the c-index determined on the five random test sets. We then used this signature to train a final Cox PH model on the target dataset.

### EXAMPLE 11

### Visualization

We employed the UMAP transform to visualize the batch integration results for each cancer type discussed in the previous examples. Specifically, we performed visualization in each experiment using the first two components of the UMAP embeddings, where the number of neighbors are set to 10 and the min_dist parameter is set to 0.5. These parameters are fixed for all visualizations in the study that employ UMAP embeddings.

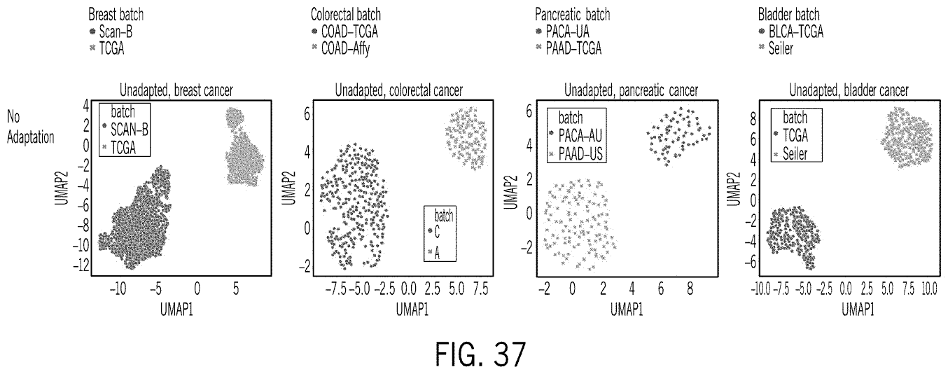

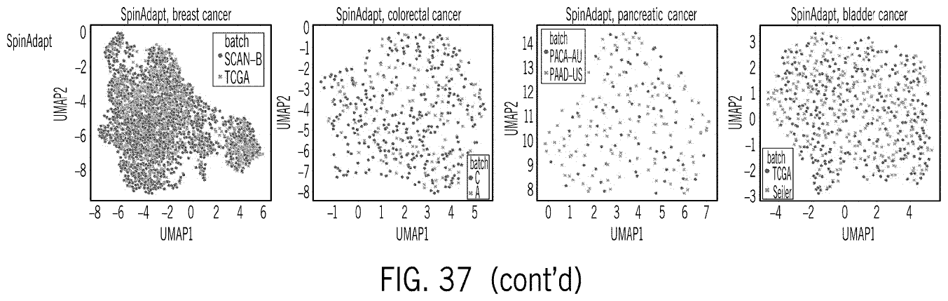

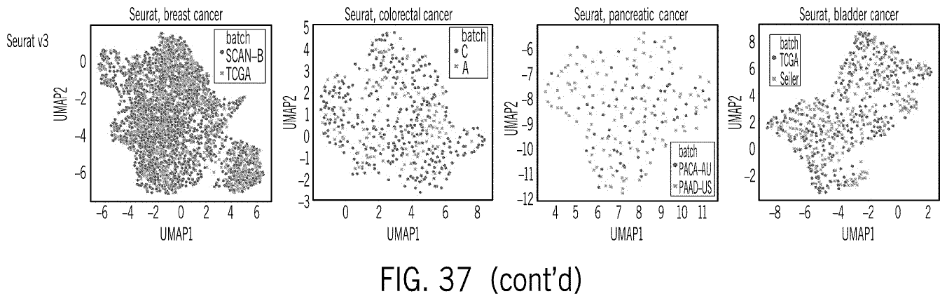

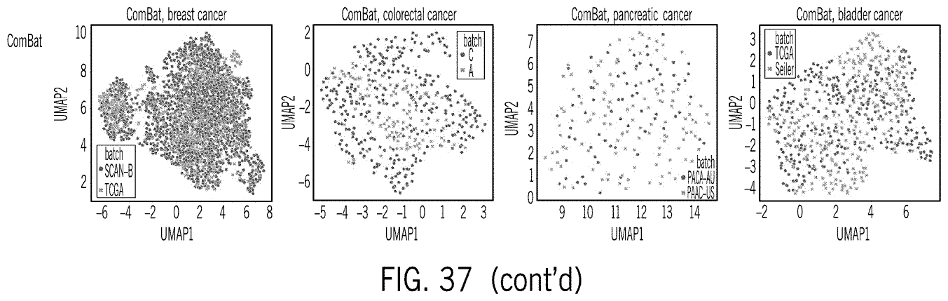

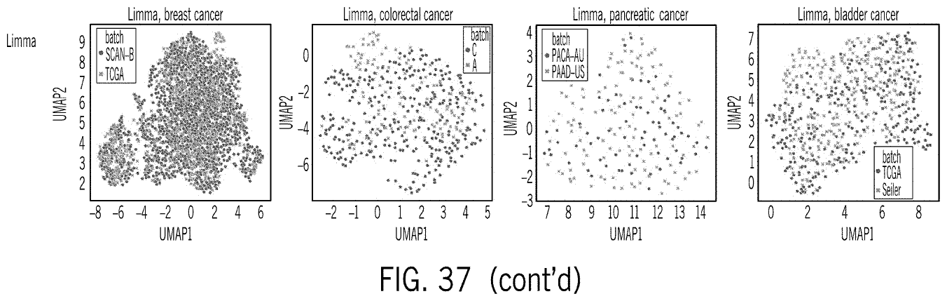

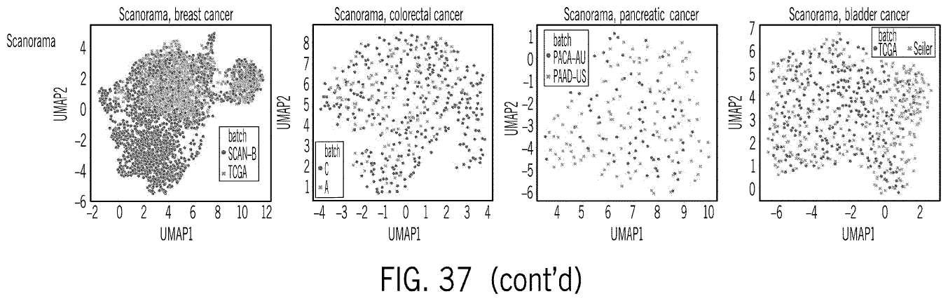

FIG. 37 includes UMAP plots for dataset integration, labeling samples by dataset. The top panel shows dataset-based clustering in each cancer dataset before integration. Integration requires good batch mixing within integrated datasets, which is achieved by most methods. ComBat, Limma, Scanorama are outperformed by Seurat and SpinAdapt in terms of batch mixing in the Colorectal batch, while Scanorama achieves poor batch mixing in Breast.

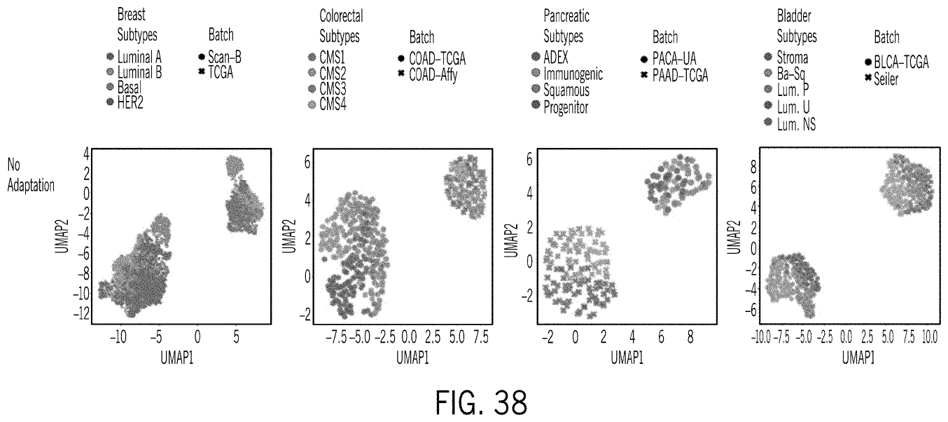

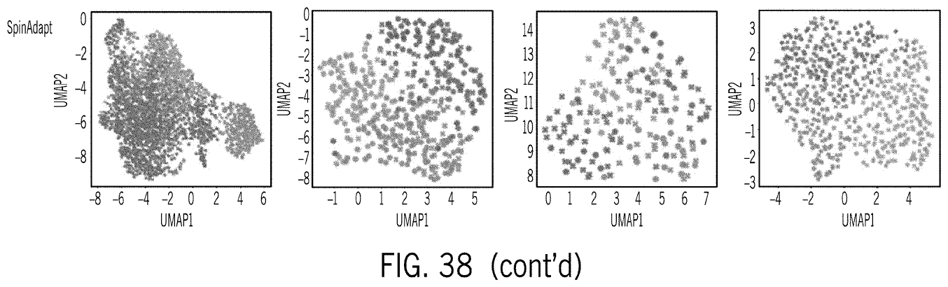

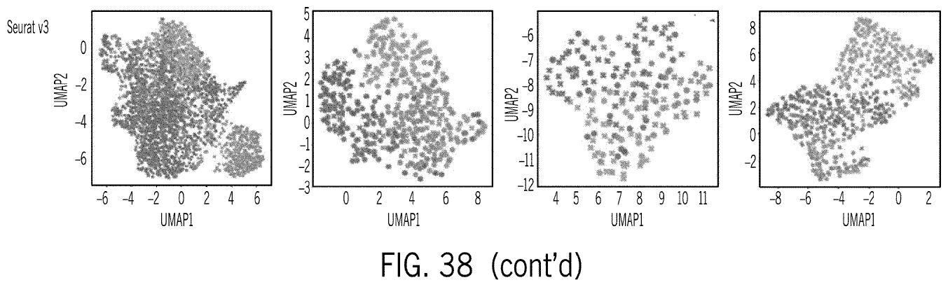

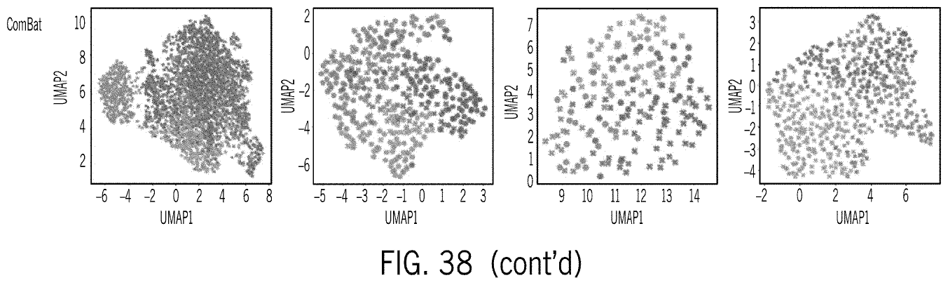

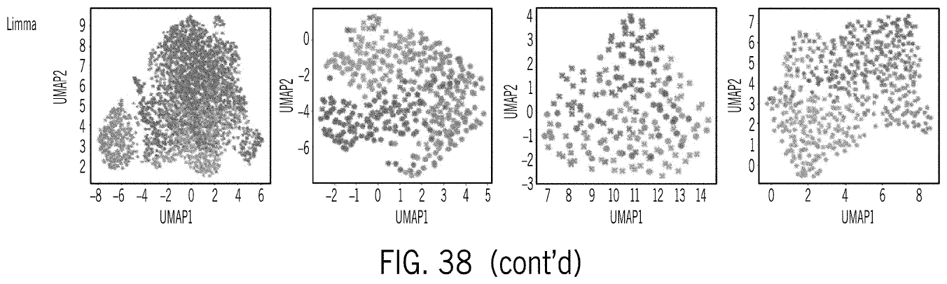

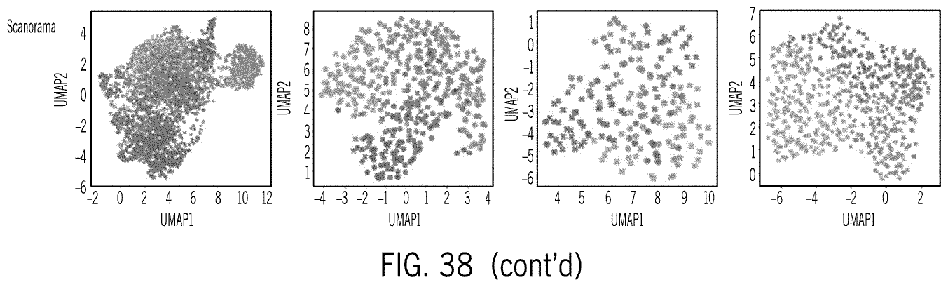

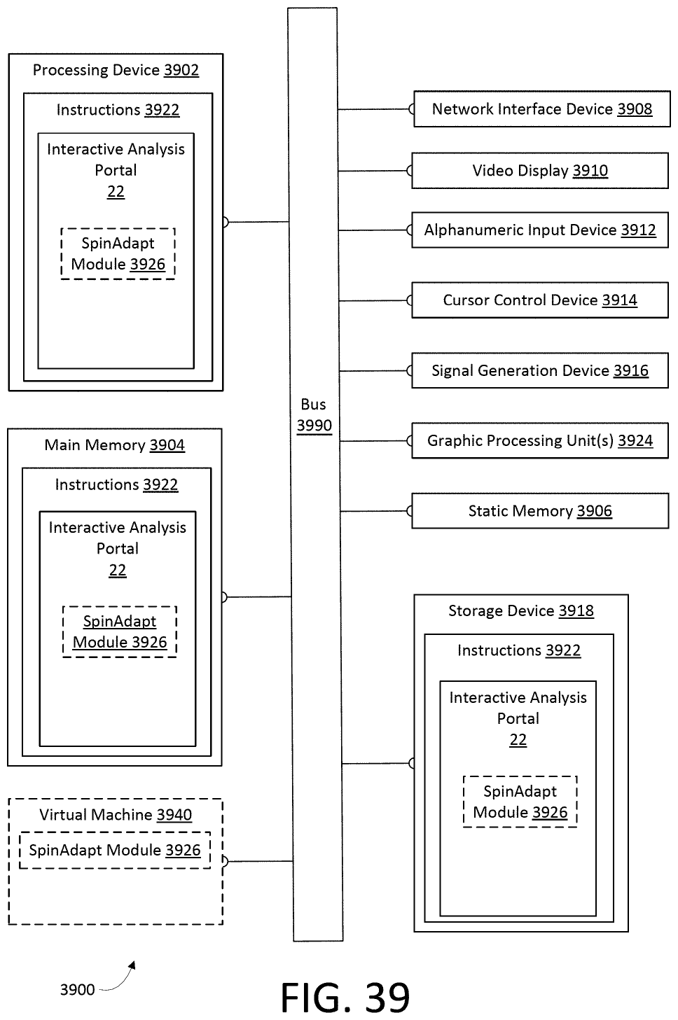

FIG. 38 includes UMAP plots for dataset integration, labeling samples by cancer subtype. The top panel shows cancer subtypes in each dataset before correction. Subtype homogeneity is apparent in the majority of integration tasks regardless of library size. Subtype mixing is visible in regions where multiple subtypes cluster together. Subtype mixing was observed before and after correction in Breast between luminal subtypes, in Colorectal between CMS 2 and 4, in Pancreatic between ADEX and immunogenic, and in Bladder between luminal subtypes.

In the traditional paradigm of sharing data models across laboratories, the discussion presumes simultaneous transfer of molecular models and their associated training datasets. Therefore, all existing batch correction methods require concurrent access to patient-level samples across all datasets. However, privacy-preserving laboratories cannot share patient-level data, and they would require a common trusted broker to have access to their private datasets. Such trusted brokers are quite common in transactional domains such as banking where privacy and trust play a major role. Recently, the use of cryptography and distributed computing has allowed the emergence of a secure, trustless financial transaction system that eliminates the use of such brokers. Similar trust limitations still exist between healthcare organizations that actively limit data sharing due to privacy and security concerns.

SpinAdapt eliminates the need for a trusted broker, as it only operates on privacy-preserving aggregate statistics of each dataset, and allows the application of a target model on privately-held source data. Besides an inherent tradeoff between performance and privacy, SpinAdapt shows state of the art performance on diagnostic, prognostic, as well as integration tasks, outperforming similar correction algorithms that require access to private sample-level data. By only sharing data factors of the training dataset alongside the assay, e.g., RNA-based data model, SpinAdapt allows external validation and reuse of pre-trained RNA models on novel datasets. The ability to share assay models such as RNA models without the necessity of sharing model training data would improve research reproducibility across laboratories and pharmaceutical partners that cannot share patient data.

The present disclosure demonstrates the application of SpinAdapt for transfer of diagnostic and prognostic models across distinct transcriptomic datasets, profiled across various laboratories and technological platforms, entailing RNA-Seq as well as various microarray platforms. Since the correction paradigm does not require sample-level data access, SpinAdapt enables correction of new prospective samples not included in training. The ability to correct held-out data is deemed necessary for validation frameworks, where the validation data needs to be completely held-out from training of any data models including classifiers, regressors, and batch correctors to avoid overfitting. SpinAdapt enables rigorous validation of molecular predictors across independent studies by holding-out the validation data from training of the predictor as well as the batch corrector.

Conversely, one advantage of the present system is the ability to eliminate a held out dataset separate from a testing or validation set. In particular, other systems may require dividing a dataset into a training subset, a testing or validation subset, and then a held out subset. This requirement may be disadvantageous, however, when the dataset is small, as the held out subset has the effect of reducing the size of the training and/or testing datasets, which may reduce the accuracy of the model. The SpinAdapt model helps alleviate this disadvantage by permitting a user to divide a dataset into a training subset and a testing subset and then using the testing subset as a validation subset.

In future work, the application of SpinAdapt can be empirically demonstrated in multiple OMICs data types, besides transcriptomics, for transfer of molecular predictors across technically biased datasets. The salient feature of SpinAdapt is the dependence on data factors from each dataset, which comprises the PCA factors of each dataset. Therefore, if PCA factors provide a sufficient representation of the considered OMICs dataset, SpinAdapt would be applicable for correction of technical biases across data acquisition platforms. PCA representations are common in various OMICs data modalities such as: Copy Number Variations (CNVs), DNA Methylation, and single-cell RNA-Seq. For cases where PCA does not provide a sufficient representation of the considered data modality, PCA can be substituted with other matrix factorization methods, such as NMF, ZIFA, and pCMF, while the rest of the algorithmic details in SpinAdapt would remain unchanged.

Appendix to the Specification

Below are simulation results for the spin adaptation pipeline in four different data settings: (a) when the number of patients in the datasets are fewer than the number of genes (patients<genes), (b) when the number of patients in the target datasets are fewer than the number of patients in the source dataset (target patients<source patients), (c) when the number of patients in the target datasets are greater than the number of patients in the source dataset (target patients>source patients), and (d) when the number of patients in the datasets are greater than the number of genes (patients>genes).

Setting (a) according to one embodiment, Setting: Patients<Genes, Genes: 4 [ADAM6, XIST, RPL21, RPS4Y1], Target patients: 3, Source patients: 3.

Target Data

TABLE 8

| | ADAM6 | XIST | RPL21 | RPS4Y1 |
|---|---|---|---|---|
| SA295845 | 15.548473 | 11.107766 | 5.352646 | 3.655629 |
| SA284604 | 12.671440 | 1.938980 | 3.571899 | 12.783485 |
| SA290474 | 15.839765 | 11.174484 | 3.622105 | 2.605884 |

Source Data

TABLE 9

| | ADAM6 | XIST | RPL21 | RPS4Y1 |
|---|---|---|---|---|
| GTEX-11ZVC- | 0.609077 | 14.854169 | 14.663797 | 3.258838 |
| 0226-SM- | | | | |
| 731E8 | | | | |
| GTEX-18A7A- | 0.609077 | 14.399653 | 14.880825 | 3.936620 |
| 1026-SM- | | | | |
| 7LT8B | | | | |
| GTEX-14E6E- | 2.362130 | 3.705443 | 14.212581 | 13.280797 |
| 0426-SM- | | | | |
| 73KUE | | | | |

Target Basis
array([[−0.22093235, −0.06663295, 0.47554612],
[−0.67016942, 0.22644422, 0.53822495],
[−0.06152513, 0.93191297, −0.2844186 ],
[ 0.70588703, 0.27535665, 0.63504004]])
Source Basis
array([[ 0.11891468, −0.27614214, 0.05085628],
[−0.74205381, 0.3623105 , −0.51703031],
[−0.03738598, 0.73662644, 0.64984017],
[ 0.65864841, 0.4998581 , −0.55479821]])
Target Embeddings
array([[−4.19648924e+00, 9.78889736e−01, 8.23317409e−17],
[ 9.13657667e+00, −5.17097359e−02, 1.03375907e−15],
[−4.94008743e+00, −9.27179996e−01, 9.60115153e−16]])
Corrected Target Embeddings
array([[−3.96621645, 0.37744722, 0.72965134],
[ 8.57401766, 1.10085682, −0.897443 ],
[−4.60780121, −1.47830405, 0.16779166]])

Corrected Target Dataset

TABLE 10

| | ADAM6 | XIST | RPL21 | RPS4Y1 |
|---|---|---|---|---|
| SA295845 | 0.654665 | 13.689069 | 15.486209 | 3.996937 |
| SA284604 | 1.863371 | 5.486896 | 14.492912 | 13.520854 |
| SA290474 | 1.062248 | 13.783300 | 13.778081 | 2.958465 |

Setting (b) according to one embodiment, Setting: Target Patients<Source Patients, Genes: 4 [ADAM6, XIST, RPL21, RPS4Y1], Target patients: 3, Source patients: 4.

Target Data

TABLE 11

| | ADAM6 | XIST | RPL21 | RPS4Y1 |
|---|---|---|---|---|
| SA279979 | 14.122654 | 13.955087 | 4.733494 | 3.244445 |
| SA280979 | 14.980531 | 2.822345 | 4.878444 | 12.909834 |
| SA277621 | 15.407801 | 1.938980 | 4.485925 | 12.658644 |

Source Data

TABLE 12

| | ADAM6 | XIST | RPL21 | RPS4Y1 |
|---|---|---|---|---|
| GTEX-1GMR3- | 0.609077 | 2.618432 | 14.453688 | 13.055414 |
| 0226-SM- | | | | |
| 7RHIB | | | | |
| GTEX-1A8G6- | 1.629993 | 3.196367 | 14.837698 | 13.201248 |
| 0726-SM- | | | | |
| 73KV5 | | | | |
| GTEX-1B933- | 1.464969 | 14.776637 | 14.610413 | 2.837371 |
| 0926-SM- | | | | |
| 9OSVH | | | | |
| GTEX-18D9A- | 1.780272 | 13.897519 | 14.615181 | 5.187002 |
| 0226-SM- | | | | |
| 7KFSJ | | | | |

$$Target Basis$$

$$array ( [ [ \begin{matrix}- 0.07196 \; 799 , & - 0.41458077 , & - 0.66366 \; 943\end{matrix} ] , \\ [ \begin{matrix}0.77054511 , & 0.48853663 , & - 0.40881297\end{matrix} ] , \\ [ \begin{matrix}0.00412909 , & 0.4176096 , & 0.46571563\end{matrix} ] , \\ [ \begin{matrix}- 0.63329598 , & 0.64424915 , & - 0.41895561\end{matrix} ] ] )$$

$$Source Basis$$

$$array ( [ [ \begin{matrix}0.03336979 , & 0.58043365 , & 0.7066924 , & 0.40334429\end{matrix} ] , \\ [ \begin{matrix}0.77761611 , & 0.48769432 , & - 0.38683509 , & - 0.08846495\end{matrix} ] , \\ [ \begin{matrix}- 0.00190051 , & 0.15086924 , & 0.3937488 , & - 0.90675065\end{matrix} ] , \\ [ \begin{matrix}- 0.62785033 , & 0.6344206 , & - 0.44274584 , & - 0.08538494\end{matrix} ] ] )$$

$$Target Embeddings$$

$$array ( [ [ \begin{matrix}1.00249707 e + 01 , & - 1.72120117 e - 02 , & - 1.11664739 e \text{-} 15\end{matrix} ] , \\ [ \begin{matrix}- 4.73550227 e + 00 , & 4.75827488 e - 01 , & 4.20646095 e - 16\end{matrix} ] , \\ [ \begin{matrix}- 5.28946839 e + 00 , & - 4.58615476 e - 01 , & 2.58011039 e - 16\end{matrix} ] ] )$$

$$Corrected Target Embeddings$$

$$array ( [ [ \begin{matrix}9.96944555 , & - 0.68110711 , & - 0.66041806 , & - 04587259\end{matrix} ] , \\ [ \begin{matrix}- 4.72761983 , & 0.54124831 , & 0.03006473 , & - 0.08456159\end{matrix} ] , \\ [ \begin{matrix}- 5.24182572 , & 0.13985881 , & 0.63035333 , & 0.54328749\end{matrix} ] ] )$$

Corrected Target Dataset

TABLE 13

| | ADAM6 | XIST | RPL21 | RPS4Y1 |
|---|---|---|---|---|
| SA279979 | 0.656737 | 16.338522 | 14.663451 | 2.210396 |
| SA280979 | 1.514613 | 5.205780 | 14.808402 | 11.875785 |
| SA277621 | 1.941883 | 4.322414 | 14.415882 | 11.624595 |

Setting (c) according to one embodiment, Setting: Target Patients>Source Patients, Genes: 4 [ADAM6, XIST, RPL21, RPS4Y1], Target patients: 4, Source patients: 3.

Target Data

TABLE 14

| | ADAM6 | XIST | RPL21 | RPS4Y1 |
|---|---|---|---|---|
| SA294017 | 14.793961 | 1.938980 | 3.645734 | 12.830224 |
| SA288271 | 15.804094 | 1.938980 | 6.061598 | 13.906181 |
| SA286781 | 15.306835 | 2.779356 | 4.691256 | 12.888387 |
| SA295679 | 13.866432 | 3.265991 | 4.681004 | 12.880658 |

Source Data

TABLE 15

| | ADAM6 | XIST | RPL21 | RPS4Y1 |
|---|---|---|---|---|
| GTEX-P4QT- | 0.609077 | 13.491811 | 14.337518 | 2.631114 |
| 0526-SM- | | | | |
| 2I3EX | | | | |
| GTEX-ZC5H- | 1.785649 | 14.486637 | 14.402374 | 5.261699 |
| 0926-SM- | | | | |
| 5CVMZ | | | | |
| GTEX-WHPG- | 2.295608 | 3.939329 | 13.903728 | 13.109975 |
| 1426-SM- | | | | |
| 3NMBB | | | | |

Target Basis
array([[ 0.55436944, 0.45088518, 0.68772411, 0.12811178],
[−0.26520143, −0.66786101, 0.58022477, 0.38336549],
[ 0.68655937, −0.58934212, −0.08949737, −0.41629596],
[ 0.3885601 , −0.05779364, −0.42704215, 0.81444211]])
Source Basis
array([[ 0.08549209, 0.38214883, 0.53444297],
[−0.72755444, 0.6531255 , −0.15992467],
[−0.03366477, 0.036903871 0.80586299],
[ 0.67986934, 0.652707791 −0.19844307]])
Target Embeddings
array([[−8.25703051e−01, 9,74387596e−01, −1.89700786e−01,
4.10237911e−6], [ 1.81039185e−00, −5,61126872e−02, −1.70700743e−01,
3.01245472e−16], [−2.38378262e−02, 2.48483310e−02, 5.32212607e−01,
2.33714624e−16], [−9.61450977e−01, −943123240e−01, −1.71811078e−01,
3.35008349e−16]])
Corrected Target Embeddings
array([[ 0.21800527, −0.6455622 , −0.84006314],
[ 0.95454427, 0.53189902, 1.43313458],
[−0.34522194, 0.17585024, 0.13064731],
[−0.8273276 , −0.06218706, −0.72371876]])

Corrected Target Dataset

TABLE 16

| | ADAM6 | XIST | RPL21 | RPS4Y1 |
|---|---|---|---|---|
| SA294017 | 0.886416 | 10.193272 | 13.506401 | 6.894486 |
| SA288271 | 2.614244 | 10.062889 | 15.356945 | 7.712674 |
| SA286781 | 1.670956 | 10.984295 | 14.337935 | 6.855076 |
| SA295679 | 1.082164 | 11.316220 | 13.656879 | 6.541482 |

Setting (d) according to one embodiment, Setting: Patients>Genes, Genes: 4 [ADAM6, XIST, RPL21, RPS4Y1], Target patients: 5, Source patients: 5.

Target Data

TABLE 17

| | ADAM6 | XIST | RPL21 | RPS4Y1 |
|---|---|---|---|---|
| SA275529 | 16.499092 | 3.755603 | 5.692487 | 13.236636 |
| SA270609 | 14.888611 | 12.154455 | 3.501123 | 3.375610 |
| SA282042 | 16.274567 | 2.646260 | 4.181123 | 13.016765 |
| SA288141 | 14.903739 | 14.251265 | 5.395963 | 3.576382 |
| SA293268 | 15.217496 | 12.906420 | 4.649091 | 2.865653 |

Source Data

TABLE 18

| | ADAM6 | XIST | RPL21 | RPS4Y1 |
|---|---|---|---|---|
| GTEX-WH7G- | 1.532710 | 3.985508 | 14.559472 | 13.496745 |
| 0726-SM- | | | | |
| 3NMBM | | | | |
| GTEX-ZPUI- | 1.555183 | 4.502236 | 14.246157 | 13.025646 |
| 0926-SM- | | | | |
| 57WDO | | | | |
| GTEX-RM2N- | 0.609077 | 3.945590 | 13.826184 | 12.913188 |
| 0426-SM- | | | | |
| 2TF4T | | | | |
| GTEX-SJXC- | 0.609077 | 3.309555 | 14.192652 | 13.053881 |
| 1026-SM- | | | | |
| EYYVA | | | | |
| GTEX-1B932- | 0.609077 | 14.629136 | 13.805651 | 4.639881 |
| 0726-SM- | | | | |
| 731EY | | | | |

Target Basis
array([[ 0.09760585, 0.08004177, 0.49542806, −0.85942856],
[−0.70898363, 0.45054869, −0.45245756, −0.29938312],
[ 0.02275206, 0.78323921, 0.50259802, 0.36525869],
[ 0.69806708, 0.42087472, −0.5451866, −0.19580191]])
Source Basis
array([[−0.03236735, 0.7899297, 0.08796582, 0.60599129],
[ 0.78363262, 0.34366111, 0.26490239, −0.44457134],
[−0.0299737, 0.34040159, −0.88476383, −0.3168932 ],
[−0.61965623, 0.37687514, 0.37320464, −0.57854091]])
Target Embeddings
array([[ 8.13842165e+00, 9.72842842e−01, 1.27903799e−01,
−7.91030636e−03], [−4.90693426e+00, −1.23858948e+00,
−1.95373377e−01, −7.89859947e-03], [ 8.71514114e+00,
−8.21238899e−01, −1.21139055e−01, 8.18386342e−03], [−6.20879832e+00,
1.27594844e+00, −2.93711820e−01, 4.14640778e−03], [−5.73783021e+00,
−1.88962899e−01, 4.82320453e−01, 3.47863464e−03]])
Corrected Target Embeddings
array([[−8.01414996, 1.50606037, 0.0111039, −0.83774188],
[ 4.79572024, −1.34206645, 0.35297122, 0.85186694],
[−8.69465421, 0.35012896, 0.95262863, 0.12552534],
[ 6.25715473, 0.11114701, −0,69180474, −0,78776399],
[ 5.6559292, −0.62526989, −0.62489901, 0.64811358]])

Corrected Target Dataset

TABLE 19

| | ADAM6 | XIST | RPL21 | RPS4Y1 |
|---|---|---|---|---|
| SA275529 | 1.925416 | 0.687208 | 15.134553 | 17.448295 |
| SA270609 | 0.314935 | 9.086059 | 12.943189 | 7.587269 |
| SA282042 | 1.700891 | −0.422135 | 13.623189 | 17.228424 |
| SA288141 | 0.330062 | 11.182869 | 14.838029 | 7.788041 |
| SA293268 | 0.643820 | 9.838025 | 14.091157 | 7.077312 |

FIG. 39 is an illustration of an example machine of a computer system 3900 within which a set of instructions, for causing the machine to perform any one or more of the methodologies discussed herein, may be executed. In alternative implementations, the machine may be connected (such as networked) to other machines in a LAN, an intranet, an extranet, and/or the Internet,

The machine may operate in the capacity of a server or a client machine in a client-server network environment, as a peer machine in a peer-to-peer (or distributed) network environment, or as a server or a client machine in a cloud computing infrastructure or environment. The machine may be a personal computer (PC), a tablet PC, a set-top box (STB), a Personal Digital Assistant (PDA), a cellular telephone, a web appliance, a server, a network router, a switch or bridge, or any machine capable of executing a set of instructions (sequential or otherwise) that specify actions to be taken by that machine. Further, while a single machine is illustrated, the term “machine” shall also be taken to include any collection of machines that individually or jointly execute a set (or multiple sets) of instructions to perform any one or more of the methodologies discussed herein.

The example computer system 3900 includes a processing device 3902, a main memory 3904 (such as read-only memory (ROM), flash memory, dynamic random access memory (DRAM) such as synchronous DRAM (SDRAM) or DRAM, etc.), a static memory 3906 (such as flash memory, static random access memory (SRAM), etc.), and a data storage device 3918, which communicate with each other via a bus 3930.

Processing device 3902 represents one or more general-purpose processing devices such as a microprocessor, a central processing unit, or the like. More particularly, the processing device may be complex instruction set computing (CISC) microprocessor, reduced instruction set computing (RISC) microprocessor, very long instruction word (VM microprocessor, or processor implementing other instruction sets, or processors implementing a combination of instruction sets. Processing device 3902 may also be one or more special-purpose processing devices such as an application specific integrated circuit (ASIC), a field programmable gate array (FPGA), a digital signal processor (DSP), network processor, or the like. The processing device 3902 is configured to execute instructions 3922 for performing the operations and steps discussed herein.

The computer system 3900 may further include a network interface device 3908 for connecting to the LAN, intranet, Internet, and/or the extranet. The computer system 3900 also may include a video display unit 3910 (such as a liquid crystal display (LCD) or a cathode ray tube (CRT)), an alphanumeric input device 3912 (such as a keyboard), a cursor control device 3914 (such as a mouse), a signal generation device 3916 (such as a speaker), and a graphic processing unit 3924 (such as a graphics card).

The data storage device 3918 may be a machine-readable storage medium (also known as a computer-readable medium) on which is stored one or more sets of instructions or software 3922 embodying any one or more of the methodologies or functions described herein. The instructions 3922 may also reside, completely or at least partially, within the main memory 3904 and/or within the processing device 3902 during execution thereof by the computer system 3900, the main memory 3904 and the processing device 3902 also constituting machine-readable storage media.

In one implementation, the instructions 3922 include instructions for an interactive analysis portal and/or a software library containing methods that function as an interactive analysis portal. The instructions 3922 may further include instructions for a SpinAdapt module 3926. While the data storage device 3918/machine-readable storage medium is shown in an example implementation to be a single medium, the term “machine-readable storage medium” should be taken to include a single medium or multiple media (such as a centralized or distributed database, and/or associated caches and servers) that store the one or more sets of instructions. The term “machine-readable storage medium” shall also be taken to include any medium that is capable of storing or encoding a set of instructions for execution by the machine and that cause the machine to perform any one or more of the methodologies of the present disclosure.

The term “machine-readable storage medium” shall accordingly be taken to include, but not be limited to, solid-state memories, optical media and magnetic media. The term “machine-readable storage medium” shall accordingly exclude transitory storage mediums such as signals unless otherwise specified by identifying the machine readable storage medium as a transitory storage medium or transitory machine-readable storage medium.

In another implementation, a virtual machine 3940 may include a module for executing instructions for a SpinAdapt module 3926. In computing, a virtual machine (VM) is an emulation of a computer system. Virtual machines are based on computer architectures and provide functionality of a physical computer. Their implementations may involve specialized hardware, software, or a combination of hardware and software,

Some portions of the preceding detailed descriptions have been presented in terms of algorithms and symbolic representations of operations on data bits within a computer memory. These algorithmic descriptions and representations are the ways used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities. Usually, though not necessarily, these quantities take the form of electrical or magnetic signals capable of being stored, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the above discussion, it is appreciated that throughout the description, discussions utilizing terms such as “identifying” or “providing” or “calculating” or “determining” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage devices.

The present disclosure also relates to an apparatus for performing the operations herein. This apparatus may be specially constructed for the intended purposes, or it may comprise a general purpose computer selectively activated or reconfigured by a computer program stored in the computer. Such a computer program may be stored in a computer readable storage medium, such as, but not limited to, any type of disk including floppy disks, optical disks, CD-ROMs, and magnetic-optical disks, read-only memories (ROMs), random access memories (RAMS), EPROMs, EEPROMs, magnetic or optical cards, or any type of media suitable for storing electronic instructions, each coupled to a computer system bus.

The algorithms and displays presented herein are not inherently related to any particular computer or other apparatus. Various general purpose systems may be used with programs in accordance with the teachings herein, or it may prove convenient to construct a more specialized apparatus to perform the method. The structure for a variety of these systems will appear as set forth in the description below. In addition, the present disclosure is not described with reference to any particular programming language. It will be appreciated that a variety of programming languages may be used to implement the teachings of the disclosure as described herein.

The present disclosure may be provided as a computer program product, or software, that may include a machine-readable medium having stored thereon instructions, which may be used to program a computer system (or other electronic devices) to perform a process according to the present disclosure. A machine-readable medium includes any mechanism for storing information in a form readable by a machine (such as a computer). For example, a machine-readable (such as computer-readable) medium includes a machine (such as a computer) readable storage medium such as a read only memory (“ROM”), random access memory (“RAM”), magnetic disk storage media, optical storage media, flash memory devices, etc.

In the foregoing specification, implementations of the disclosure have been described with reference to specific example implementations thereof. It will be evident that various modifications may be made thereto without departing from the broader spirit and scope of implementations of the disclosure as set forth in the following claims. The specification and drawings are, accordingly, to be regarded in an illustrative sense rather than a restrictive sense.

1. A method for predicting a classification of a new patient's risk of cancer recurrence comprising:
receiving a first dataset having a first set of RNA expression data generated from a first set of sequencing equipment;
transforming the first set of RNA expression data based at least in part on characteristics of a second dataset having a second set of RNA expression data generated from a second set of sequencing equipment;
generating a molecular model trained from the transformed first set of RNA expression data;
receiving a record associated with the new patient, the record having RNA expression data generated from the second set of sequencing equipment; and
providing the RNA expression data of the record to the generated molecular model to predict the classification of the new patient's risk of cancer recurrence.

2. The method of claim 1, wherein the first sequencing equipment comprises microarray sequencing equipment and the second sequencing equipment comprises next-generation sequencing equipment. The method of claim 1, wherein the first sequencing equipment comprises next-generation sequencing equipment and the second sequencing equipment comprises microarray sequencing equipment.

4. The method of claim 1, wherein the first dataset is a public dataset and the second dataset is a laboratory-specific dataset.

5. The method of claim 1, wherein the first dataset is a laboratory-specific dataset and the second dataset is a public dataset.

6. The method of claim 1, wherein the first dataset is a first laboratory-specific dataset and the second dataset is a second laboratory-specific dataset.

7. The method of claim 1, further comprising predicting a patient's outcome to treatments based at least in part on the prediction of the new patient's risk of cancer recurrence.

8. The method of claim 1, wherein the prediction of the new patient's risk of cancer recurrence is based at least in part on a cancer type or subtype.

9. The method of claim 1, wherein the first dataset is a germ line dataset from a laboratory in a first location and the second dataset is a germ line dataset from a laboratory in a second location.

10. The method of claim 1, wherein transforming the first set of RNA expression data comprises:
generating a transform between a first set of adaptation factors of the first dataset and a second set of adaptation factors of the second dataset;
encoding two or more corresponding eigenvalues of the second dataset with the generated transform; and
providing the encoded two or more corresponding eigenvalues of the second dataset as the adapted second dataset.

11. The method of claim 1, further comprising:
wherein the record further comprises DNA mutation data generated from the second set of sequencing equipment.

12. The method of claim 1, further comprising:
receiving a second record associated with a second new patient, the second record having RNA expression data generated from a third set of sequencing equipment;
transforming RNA expression data of the second record based at least in part on characteristics of the first set of RNA expression data; and
providing the transformed RNA expression data of the second record to the generated molecular model to predict the classification of the second new patient's risk of cancer recurrence.

13. The method of claim 1, wherein the classification of the new patient's risk of cancer recurrence further comprises an estimate on a duration of time until the new patient's risk of cancer recurrence occurs.

14. The method of claim 1, wherein the first set of sequencing equipment and the second set of sequencing equipment sequences up to 140,000 transcripts.

15. The method of claim 1, wherein the first set of sequencing equipment and the second set of sequencing equipment sequences between 10 and 20,000 genes.

16. The method of claim 1, wherein characteristics of the first set of sequencing equipment sequences have differences from the second set of sequencing equipment sequences.

17. The method of claim 16, wherein the characteristics are measured by a variance of each gene.

18. The method of claim 16, wherein the characteristics are measured by heterogeneity.

19. The method of claim 16, wherein the characteristics are measured across high-expression genes.

20. The method of claim 16, wherein the characteristics occur in at least one of the FGFR2, MAP3K1, TNRC9, BRCA1, and BRCA2 genes.

21. The method of claim 1, wherein the sequencing further comprises sequencing different gene expression across different cancer types.

22. The method of claim 21, wherein the different cancer types comprise at least two of breast, colorectal, pancreatic, lung, and bladder cancers.

23. The method of claim 21, wherein the different cancer types comprise at least two of squamous, immunogenic, luminal, and basal cancers.

24. The method of claim 21, wherein the different cancer types comprise at least two of adenosquamous, adenocarcinoma, and neuroendocrine cancers.

25. The method of claim 1, wherein the sequencing further comprises different types of cells selected from tumor, stroma, epithelium, fat, and lymphocytes.

26. The method of claim 1, wherein the record further comprises pathology imaging features from a pathology slide.

27. The method of claim 1, wherein a training dataset of the molecular model comprises both a subset of the first set of RNA expression data and a subset of the second set of RNA expression data.

28. The method of claim 27, wherein the training dataset of the molecular model further comprises a subset of a third set of RNA expression data generated from a third set of sequencing equipment.

29. The method of claim 27, wherein the training dataset excludes gene variants which are not informative to the molecular model.
